(19) United States
(12) Patent Application Publication
(10) Pub. No.: US 2025/0161288 A1
(43) Pub. Date: May 22, 2025

(54) MULTIMERIZER PHARMACEUTICAL COMPOSITIONS

(71) Applicant: PONCE THERAPEUTICS, INC., Surfside, FL (US)

(72) Inventor: Daniel L. Jasinski, Surfside, FL (US)

(21) Appl. No.: 18/839,281

(22) PCT Filed: Feb. 16, 2023
(86) PCT No.: PCT/US2023/062760

§ 371 Date: Aug. 16, 2024

Related U.S. Application Data
(60) Provisional application No. 63/479,333, filed on Jan. 10, 2023.
(60) Provisional application No. 63/329,595, filed on Apr. 11, 2022.
(60) Provisional application No. 63/311,698, filed on Feb. 18, 2022.

(51) Int. Cl. A61K 31/4545; A61K 9/00; A61K 9/107; A61K 9/70; A61K 47/10; A61K 47/14; A61K 47/26; A61K 47/44

(52) U.S. Cl. CPC A61K 31/4545; A61K 9/0019; A61K 9/107; A61K 9/7023; A61K 47/10; A61K 47/14; A61K 47/26; A61K 47/44

(57) ABSTRACT

The technology relates in part to formulations of rimiducid, rimiducid family compounds and analogs thereof, and in part to formulations suitable for administration by injection and topical administration.

### RELATED PATENT APPLICATIONS

This patent application claims the benefit of U.S. provisional patent application No. 63/311,698 filed on Feb. 18, 2022, U.S. provisional patent application No. 63/329,595 filed on Apr. 11, 2022, and U.S. provisional patent application No. 63/479,333 filed on Jan. 10, 2023. The entire content of the foregoing patent applications is incorporated herein by reference for all purposes.

### FIELD

The technology relates in part to formulations of rimiducid and analogs thereof, and relates in part to formulations suitable for administration by injection and topical administration.

### BACKGROUND

Chemically induced dimerization is a technology that allows for remote control of one or more signaling cascades among numerous signaling pathways within cells. The cells typically are genetically modified to express chimeric proteins that respond to a multimerizing ligand (also referred to herein as a “multimerizer” and “multimerizing agent”). There is a relatively small number of ligands suited for use in humans expressing multimerizable chimeric proteins. One such ligand is rimiducid, also referred to as AP1903, which is a homodimerizer having two identical binding sites and is 1411 Daltons in size. Rimiducid homodimerizes an analog of the human, prolyl isomerase protein, FKBP12, which can be genetically engineered into fusion proteins that are activated upon rimiducid-induced dimerization of the FKBP12 portions. This chemical-induced dimerization (CID) technology can be used to trigger a wide spectrum of downstream signaling cascades, and the use of rimiducid as a chemical dimerizer to trigger the caspase 9 apoptotic pathway has been clinically validated.

### SUMMARY

Described herein are pharmaceutical compositions for injection (e.g., subcutaneous (SQ), intramuscular (IM)) or for topical administration that include an active ingredient containing a multimerizer. Non-limiting examples of a multimerizer active ingredient that can be included in such a pharmaceutical composition include a compound having a structure according to Formula A, a compound having a structure according to Formula B, a rimiducid analog (e.g., a compound having a structure according to Formula C, D, E, F or G, or a pharmaceutically acceptable salt, ester or amide thereof), as described herein.

In certain aspects, provided is a pharmaceutical composition for administration by injection, such as subcutaneous (SQ) or intramuscular (IM) administration. In certain implementations, a pharmaceutical composition for administration by injection includes a multimerizer as an active ingredient and includes an oil component. In certain instances, a pharmaceutical composition for administration by injection includes a multimerizer active ingredient, includes an oil component, includes no aqueous component, and optionally includes a solvent component, or a surfactant component, or a solvent component and a surfactant component. In certain implementations, a pharmaceutical composition for administration by injection includes a multimerizer active ingredient and (i) an aqueous component, a solvent component and a surfactant component, or (ii) an aqueous component, a surfactant component and an oil component, or (iii) an aqueous component, a solvent component, a surfactant component and an oil component. An aqueous component, a solvent component, a surfactant component and an oil component are referred to herein as an aqueous excipient, a solvent excipient, a surfactant excipient and an oil excipient, respectively. A surfactant component sometimes is a solubility enhancer component.

An oil component can include one or more of medium-chain triglycerides (MCT), castor oil (CO), medium-chain triglycerides of caprylic (C8) and capric (C10) acids (CAP), oleoyl polyoxyl-6 glycerides (OPG), polyethoxylated castor oil (PECO), Triacetin, oleic acid (OA), propylene glycol monocaprylate (PGMC) (Type II), glycerol monooleate (GMO), caprylocaproyl polyoxyl-8 glycerides (CCPG), propylene glycol monolaurate (PGML) Type II, glyceryl monolinoleate (GML) and Pluronic® polymers (PP). An oil component sometimes is MCT or CO. A MCT oil component sometimes includes one or more of C6, C8, C10 and C12 fatty acids (for example, C8 and C10 fatty acids and optionally a C12 fatty acid), sometimes includes one or more of C6, C8, C10 and C12 saturated fatty acids (for example, C8 and C10 saturated fatty acids and optionally a C12 saturated fatty acid), and sometimes is from a plant (e.g., coconut). An aqueous component often is water, a solvent component sometimes is a polyethylene glycol (PEG) (for example, PEG400), and a surfactant component sometimes is a polysorbate (PS) (for example, PS80).

In certain aspects, provided is a pharmaceutical composition for topical administration (e.g., transdermal administration, transmucosal administration). In certain implementations, a pharmaceutical composition includes a multimerizer active ingredient, and a solubilizing excipient component that includes one or more of ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

Certain implementations are described further in the following description, examples and claims.

### DETAILED DESCRIPTION

For a compound to reach systemic circulation, it must first be in solution, then permeate the cellular absorption barriers and finally escape intestinal/hepatic first-pass metabolism. Rimiducid is a multimerizer characterized as a class IV compound having low aqueous solubility (i.e., a volume of greater than 250 milliliters required to dissolve an effective dose), low gastrointestinal permeability (i.e., less than 10⁻⁵ centimeters per second), high molecular weight and poor bioavailability (i.e., less than 90% of a dose administered by a non-intravenous route is absorbed, compared to an intravenous dose). Rimiducid is considered a poor candidate for oral administration and other routes of administration in view of these physical properties, as addressed in greater detail hereafter.

An API possessing all physical properties identified according to Lipinski's Rule of Five (R05) is expected to have a relatively high probability of drug absorption from the intestinal track, and thereby a relatively high probability of oral bioavailability (Lipinski et al., Adv Drug Deliv Rev. 2001 Mar. 1; 46(1-3):3-26. doi: 10.1016/s0169-409x(00)00129-0). According to the R05, an API formulated for oral administration should have no more than one violation of the identified physical properties. The physical properties according to Lipinski's R05 are: (1) no more than 5 hydrogen bond donors (the total number of nitrogen-hydrogen and oxygen-hydrogen bonds); (2) no more than 10 hydrogen bond acceptors (all nitrogen or oxygen atoms); (3) a molecular mass less than 500 Daltons (Da); and (4) an octanol-water partition coefficient (log P) that does not exceed 5.

Rimiducid, having a structure according to Formula B herein and within the family of molecules defined by the structure according to Formula A herein, violates all of the physical properties according to Lipinsky's Rule and therefore is not expected to display clinically relevant oral absorption. Rimiducid has 22 hydrogen bond donors (maximum allowed is 5), 24 hetero-atoms (0+N; maximum allowed is 10), a molecular mass of 1411.6 Da (maximum allowed is 500 Da) and a cLogP of 11.9 (maximum allowed is 5) (PubChem).

Rimiducid has an extremely low water solubility of less than 0.5 μg/mL (Drug Bank Online), likely because it possesses no ionizable functional units. As such, rimiducid is categorized as a biopharmaceutical classification system (BCS) class IV drug. Although there are exceptions, BCS class IV compounds generally are viewed as poor candidates for oral delivery (Markovic et al., Pharmaceutics 2020, 12(12), 1175. doi.org/10.3390/pharmaceutics12121175). For a compound to be absorbed, it must first be in solution. Because of the extremely low water solubility, assuming an upper gastro-intestinal (GI) tract water content of approximately 500 mLs (if the dose were taken with 240 mLs of water), the theoretical maximal solubility of rimiducid within the upper GI tract likely is limited to less than 250 μg. Once solubilized, rimiducid must cross the plasma membranes of the intestinal tract to be absorbed. However, because of the extremely high number of hydrogen bond donors (22) possessed by rimiducid, the de-solvation energy required for lipid permeation would preclude passive permeability (Milanetti et al., Bioinformatics, 2016 April:32(8); 1163-1169, doi.org/10.1093/bioinformatics/btv725). Because of the molecular weight of 1411.6 Da, passive absorption of rimiducid through the intestinal aqueous porin channels is unlikely (Dahlgren et al., Pharmaceutics. 2019; 11(8):411. Published 2019 Aug. 13. doi:10.3390/pharmaceutics11080411).

Due to the challenging physical and chemical properties of rimiducid, a limited number of formulation excipients has been identified and route of administration is limited. In a clinical setting, rimiducid typically is infused slowly by intravenous (IV) administration to patients over a timespan covering two hours or longer. Rimiducid typically is stored in 25% Solutol HS15/Kolliphor®HS15 (CAS #70142-34-6) at 5 mg/ml, which is added to a 100-mL bag of saline. Solutol HS15 is associated with toxic side-effects when dosed intravenously, which is a primary reason for long infusion times. Due to risk of an infusion reaction to Solutol HS15, patients typically are treated prophylactically with the anti-histamine diphenhydramine and other anti-inflammatory agents. The labor intensity, slow infusion rate, and potential Solutol HS15 toxicity associated with Intravenous administration of rimiducid has limited it to a once-per-week dosing regimen, reducing its widespread applicability.

It was not known whether a formulation of rimiducid and related molecules could be prepared for successful administration by injection (e.g., subcutaneous, intramuscular) or topical administration (e.g., transdermal, transmucosal) given its prohibitive physical and chemical properties, as described above. Provided herein, however, are formulations for injection (e.g., subcutaneous injection, intramuscular injection) and topical administration (e.g., transdermal) of rimiducid and related molecules. By obviating the requirement for intravenous administration and pre- and post-procedure measures to manage the possibility of infusion-based complications, the formulations provided herein can be considered for administration to a larger numbers of patients and for use in situations that could benefit from more frequent and/or longer exposure to the drug.

### Multimerizer Active Ingredient

A pharmaceutical composition generally includes one or more active pharmaceutical ingredients (APIs), each of which can be referred to as an “active ingredient”. An active ingredient in a pharmaceutical composition, in certain implementations, can include a rimiducid family molecule having a structure of Formula A.

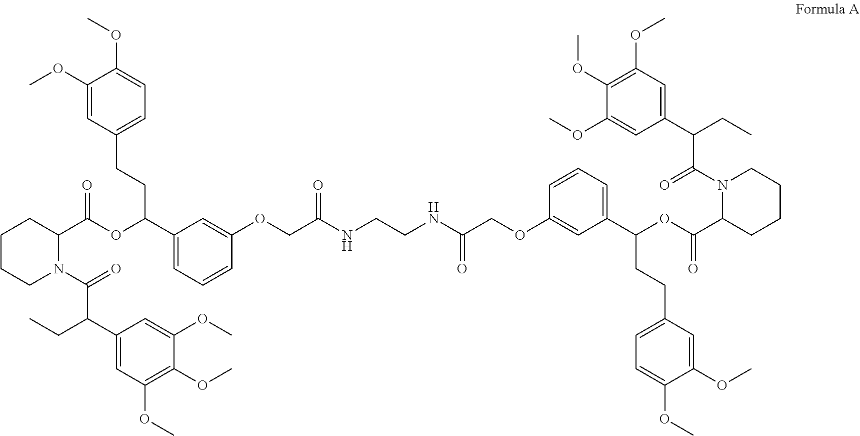 [SMILES: CCC(C(=O)N1CCCCC1C(=O)OC(CCc1ccc(OC)c(OC)c1)c1cccc(OCC(=O)NCCNC(=O)COc2cccc(C(CCc3ccc(OC)c(OC)c3)OC(=O)C3CCCCN3C(=O)C(CC)c3cc(OC)c(OC)c(OC)c3)c2)c1)c1cc(OC)c(OC)c(OC)c1]

The rimiducid family of molecules having a structure according to Formula A includes rimiducid having a structure of Formula B. The compound having the structure of Formula B also is referred to as AP1903. An active ingredient in a pharmaceutical composition, in certain implementations, includes rimiducid having a structure of Formula B.

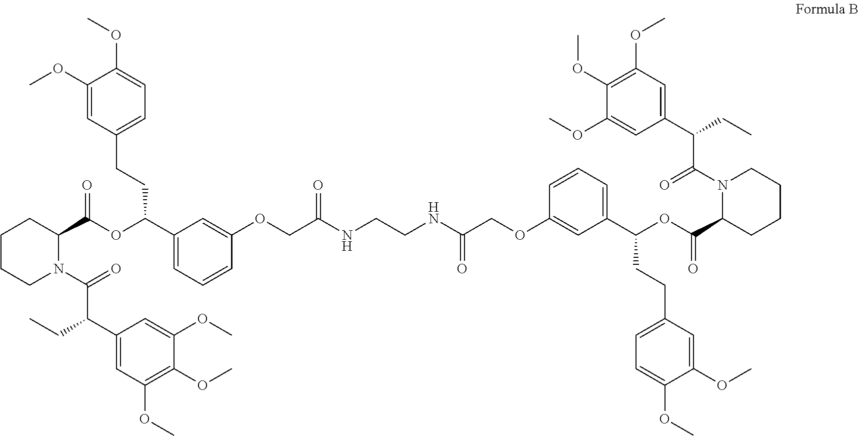 [SMILES: CC[C@H](C(=O)N1CCCC[C@H]1C(=O)O[C@H](CCc1ccc(OC)c(OC)c1)c1cccc(OCC(=O)NCCNC(=O)COc2cccc([C@@H](CCc3ccc(OC)c(OC)c3)OC(=O)[C@@H]3CCCCN3C(=O)[C@@H](CC)c3cc(OC)c(OC)c(OC)c3)c2)c1)c1cc(OC)c(OC)c(OC)c1]

An active ingredient in a pharmaceutical composition in certain instances can include a rimiducid analog. A rimiducid analog can include one or more atom modifications, one or more chemical group modifications and/or one or more linkage modifications relative to rimiducid or a rimiducid family compound. A modification may be a removal, substitution or addition, and sometime is a conservative or minor modification. A modification sometimes is in the linker or non-binding, peri-linker sides of the molecule having a structure of Formula A or Formula B that affects solubility or stability and not binding to a polypeptide (e.g., FKBP12-V36 polypeptide (e.g., polypeptide of SEQ ID NO:1 or SEQ ID NO:4). In certain instances, 1, 2, 3, 4 or 5 atoms, chemical groups and/or linkages in rimiducid or a rimiducid family compound are modified in a rimiducid analog. Non-limiting examples of rimiducid analogs include AP20187 and AP1510, structural formula for which are provided hereafter (i.e., Formula C and D). Other non-limiting examples of a rimiducid analog have a structure according to Formula E, in which two ester groups in rimiducid are substituted with amide groups, or a structure according to Formula F and Formula G, where substituents A, Y, Z, A‘and Z’ are defined in publication WO2019126344A1 (PCT/US2018/066532).

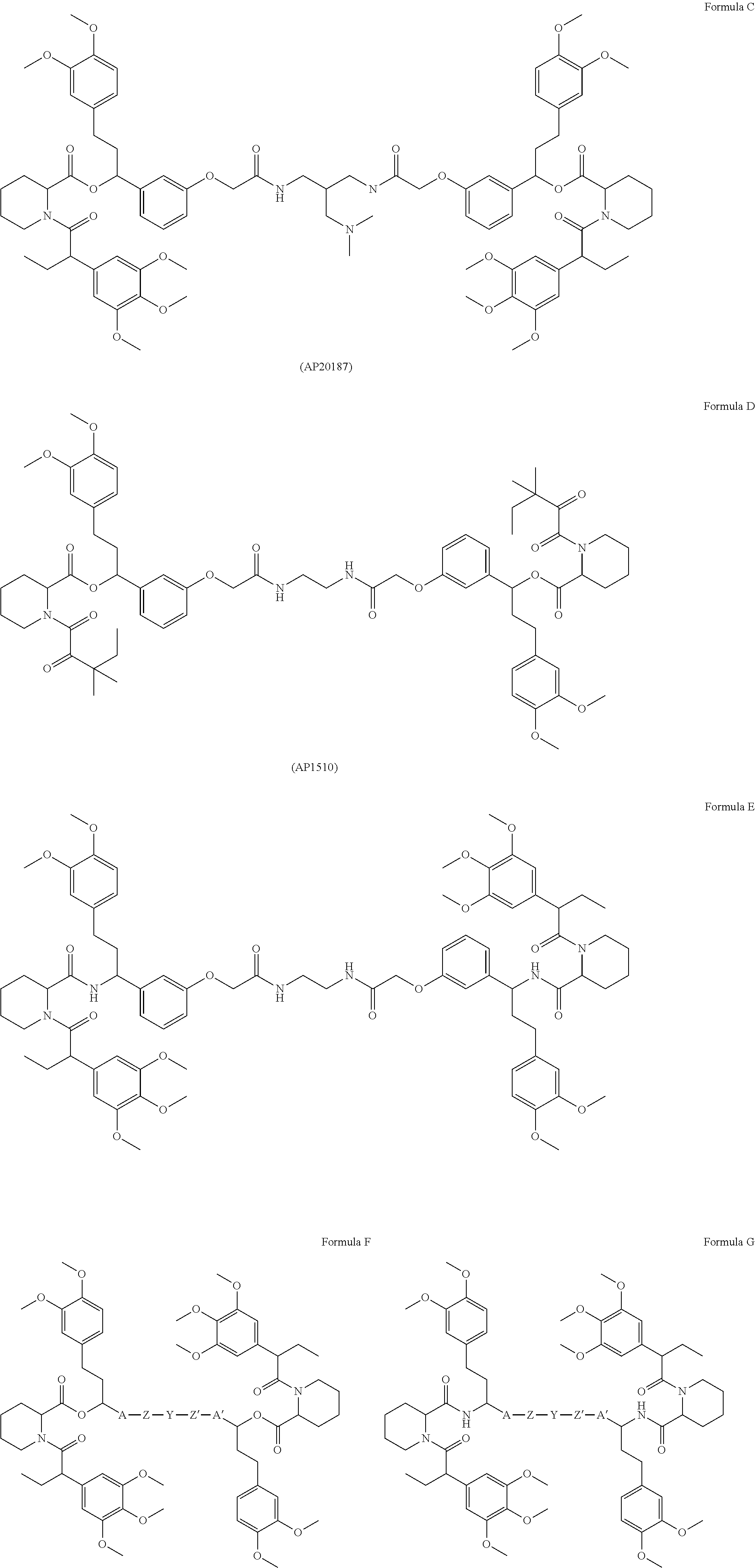

A rimiducid analog sometimes is a Class IV compound, which is characterized by low aqueous solubility, low gastrointestinal permeability and poor bioavailability (as described herein).

An active ingredient sometimes includes a pharmaceutically acceptable salt, ester or amide of a compound described herein. As used herein, the term “pharmaceutically acceptable salt” includes but is not limited to a carboxylate salt, amino acid addition salt or zwitterionic form thereof, known as suitable for use with humans and animals. (See, e.g., Gerge, S. M., et al, “Pharmaceutical Salts,” Pharm. Sci. (1977) 66:1-19). In cases where a compound (e.g., rimiducid analog) is sufficiently basic or acidic to form a stable nontoxic acid or base salt, an active ingredient sometimes includes a pharmaceutically acceptable salt of the compound. Non-limiting examples of pharmaceutically acceptable salts are organic acid addition salts formed with acids that form a physiological acceptable anion, non-limiting examples of which include tosylate, methanesulfonate, acetate, citrate, malonate, tartarate, succinate, benzoate, ascorbate, [alpha]-ketoglutarate, and [alpha]-glycerophosphate. Suitable inorganic salts may also be formed, including hydrochloride, sulfate, nitrate, bicarbonate, and carbonate salts. Pharmaceutically acceptable salts are obtained using standard procedures known in the art. For example, pharmaceutically acceptable salts may be obtained by reacting a sufficiently basic compound with a suitable acid affording a physiologically acceptable anion. Alkali metal (e.g., sodium, potassium or lithium) or alkaline earth metal (e.g., calcium, magnesium) salts of carboxylic acids and other anionic groups in molecules within a pharmaceutical composition also are contemplated. For an ester or amide of a compound, a carboxylic acid of a compound sometimes is modified to an amide or an ester, and sometimes an ester of a compound is converted to an amide.

An active ingredient in a pharmaceutical composition in certain instances can include an isomer of rimiducid having a structure according to Formula B (e.g., an isomer having a structure according to Formula A), or an isomer of a rimiducid analog (e.g., having a structure according to Formula C, D, E, F or G, or a pharmaceutically acceptable salt, ester or amide thereof). Non-limiting examples of isomers are stereoisomers (e.g., diastereomers and enantiomers), and structural isomers (e.g., skeletal isomers, positional isomers (or regioisomers), functional isomers, tautomers, and structural topoisomers). An active ingredient in a pharmaceutical composition in certain instances can include a mixture that contains two or more stereoisomers of a compound having a structure according to Formula A or Formula B, or two or more stereoisomers of a rimiducid analog. In certain instances, a mixture can include an stereoisomer of a compound having a structure according to Formula A or Formula B, or a rimiducid analog, that predominates over other stereoisomers thereof (e.g., the molar amount of one stereoisomer represents about 60% or more of all isomers of a compound having a structure according to Formula A or Formula B or a rimiducid analog (e.g., about 65% or more, about 70% or more, about 75% or more, about 80% or more, about 85% or more). An active ingredient in a pharmaceutical composition in certain instances can include an isomerically pure form of a compound having a structure according to Formula A or Formula B or a rimiducid analog. For an isomerically pure form of an active ingredient, the molar amount of one isomer represents about 90% or more (e.g., about 91% or more, about 92% or more, about 93% or more, about 94% or more, about 95% or more, about 96% or more, about 97% or more, about 98% or more, about 99% or more, about 99.5% or more or about 99.9% or more) of all isomers of the active ingredient (e.g., rimiducid having a structure of Formula B).

A compound having a structure according to Formula A or Formula B or a rimiducid analog can be present in a pharmaceutical composition described herein in a weight to weight (wt/wt) amount. A weight to weight amount typically is weight in milligrams (mg) of a compound of Formula A or Formula B or a rimiducid analog divided by the weight in grams (g) of the pharmaceutical composition. In certain implementations, a compound having a structure according to Formula A or Formula B or a rimiducid analog can be present in a pharmaceutical composition described herein in a weight to weight (wt/wt) amount of about 5 mg/g to about 200 mg/g, or about 10 mg/g or greater, about 15 mg/g or greater, about 20 mg/g or greater, about 25 mg/g or greater, about 30 mg/g or greater, about 35 mg/g or greater, about 40 mg/g or greater, about 45 mg/g or greater, about 50 mg/g or greater, about 55 mg/g or greater, about 60 mg/g or greater, about 65 mg/g or greater, about 70 mg/g or greater, about 75 mg/g or greater, about 80 mg/g or greater, about 85 mg/g or greater, about 90 mg/g or greater, about 95 mg/g or greater, about 100 mg/g or greater, about 105 mg/g or greater, about 110 mg/g or greater, about 115 mg/g or greater, about 120 mg/g or greater, about 125 mg/g or greater, about 130 mg/g or greater, about 140 mg/g or greater, about 150 mg/g or greater, about 160 mg/g or greater, about 170 mg/g or greater, about 180 mg/g or greater, or about 190 mg/g or greater. In certain implementations, a compound having a structure according to Formula A or Formula B or a rimiducid analog is present in a pharmaceutical composition described herein at a concentration of about 5 millimolar (mM) to about 140 mM. In certain implementations, an active ingredient (e.g., a compound having a structure according to Formula A or Formula B) is present in a pharmaceutical composition described herein in an amount of about 10 mM or greater, about 15 mM or greater, about 20 mM or greater, about 25 mM or greater, about 30 mM or greater, about 35 mM or greater, about 40 mM or greater, about 45 mM or greater, about 50 mM or greater, about 55 mM or greater, about 60 mM or greater, about 65 mM or greater, about 70 mM or greater, about 75 mM or greater, about 80 mM or greater, about 85 mM or greater, about 90 mM or greater, about 95 mM or greater, about 100 mM or greater, about 105 mM or greater, about 110 mM or greater, about 115 mM or greater, about 120 mM or greater, about 125 mM or greater, or about 130 mM or greater.

### Additives

A pharmaceutical composition can include an active ingredient and one or more pharmaceutical additives, including one or more pharmaceutically acceptable excipients. One or more pharmaceutically acceptable excipients in a pharmaceutical composition can form a carrier for the active ingredient. Pharmaceutical compositions provided herein include additives suitable for an injection route of administration (e.g., subcutaneous (SQ) or intramuscular (IM) route of administration) or topical route of administration (e.g., transdermal or transmucosal (e.g., intranasal, buccal, sublingual, vaginal or rectal) route of administration).

One or more additives can be combined with an active ingredient for the manufacture of a pharmaceutical composition by methods known in the art and described herein. Additives typically are pharmaceutically acceptable additives and non-limiting examples of additives include an oil component, surfactant component, solvent component, aqueous component, penetration enhancer component, solubility enhancer component, diluent, isotonic agent, buffering agent, stabilizer, preservative, vasoconstrictive agent, antibacterial agent, antifungal agent, adsorption delaying agents and the like). A pharmaceutical composition sometimes is prepared as a solid (e.g., powder) or liquid (e.g., aqueous solution, emulsion (e.g., micro-emulsion, nano-emulsion)), for example.

Non-limiting examples of solvents and diluents include water, saline, dextrose, ethanol, glycerol, oil, water-miscible organic cosolvents such as acetone or dimethyl sulfoxide (DMSO), and the like. A solvent component (also referred to as a solvent excipient) utilized sometimes includes a polyethylene glycol (PEG), such as PEG 300 or PEG400, for example. The PEG in PEG300 typically has an average molecular weight of 300 g/mol. The PEG in PEG400 typically has an average molecular weight of 400 g/mol and PEG-8 (PEG chains with 400 MW) often predominates. A PEG300 component or PEG400 component typically is not a mixture with non-PEG compounds (e.g., not typically a mixture of monoesters and diesters and/or diglycerides and triglycerides). A solvent component sometimes is a non-oil solvent component. A solvent component can include one or more polar solvents, non-limiting examples of which include N-methyl 2-pyrrolidone (NMP), polyethylene glycol 300 (PEG300), polyethylene glycol 400 (PEG400), DMSO, 2-(2-ethoxyethoxy)ethanol (EEE) and ethanol. The polar solvent EEE has a structure according to the formula CH₃CH₂OCH₂CH₂OCH₂CH₂OH), and is commercially available under the tradename Transcutol® and Transcutol® HP.

Non-limiting examples of isotonic agents include sodium chloride, dextrose, mannitol, glucose, sucrose, sorbitol, lactose, and the like. Non-limiting examples of buffering agents include bicarbonate, phosphate, and the like. Phosphate-buffered saline (PBS), which may be buffered to provide a neutral pH, or in certain embodiments an acidic pH, sometimes is utilized. Non-limiting examples of stabilizers include gelatin, albumin, and the like. Non-limiting examples of a preservatives include gentamicin, Merthiolate, chlorocresol and the like.

Water or saline used for preparing a pharmaceutical composition may be buffered or not buffered. Non-limiting examples of saline solutions that can be used to prepare a pharmaceutical composition include lactated Ringer's solution, acetated Ringer's solution, intravenous sugar solutions (e.g., 5% dextrose in normal saline (D5NS), 10% dextrose in normal saline (D10NS), 5% dextrose in half-normal saline (D5HNS) and 10% dextrose in half-normal saline (D10HNS)). Non-limiting example of buffered saline solutions and related solutions include phosphate buffered saline (PBS), TRIS-buffered saline (TBS), Hank's balanced salt solution (HBSS), Earle's balanced salt solution (EBSS), standard saline citrate (SSC), HEPES-buffered saline (HBS), and Gey's balanced salt solution (GBSS).

A pharmaceutical composition can include an excipient component that enhances solubility of an active ingredient. Such a “solubilizing excipient component” can enhance solubility of rimiducid or a rimiducid analog relative to solubility of rimiducid or the rimiducid analog without the solubilizing excipient component. A solubilizing excipient component may exhibit other features, such as enhancing bioavailability of an active ingredient (e.g., a compound having a structure according to Formula A or Formula B, or a rimiducid analog (e.g., a compound having a structure according to Formula C, D, E, F or G)), for example. In certain implementations, an excipient additive includes one or more of the following: lipid, polyethylene glycol (PEG), polysorbate, glycerol, glycerin, dimethylacetamide, triacetin, and an oil (e.g., a vegetable oil), and combinations thereof. Various sustained release agents for drugs have also been devised, and can included in a pharmaceutical composition (see, e.g., U.S. Pat. No. 5,624,677). In some embodiments, solubility of an active ingredient is characterized by a particular amount of excipient that confers solubility to the agent. In some embodiments, an active ingredient is characterized as requiring at least about 1% excipient for solubility in water. For example, an active ingredient may be characterized as requiring at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% total solubility enhancing excipient (e.g., non-aqueous component excipient) for solubility in water.

### Pharmaceutical Compositions for Administration by Injection

Certain pharmaceutical compositions provided herein are for administration by injection, including subcutaneous (SQ) administration or intramuscular (IM) administration, and each is referred to herein as a “pharmaceutical composition for injection.” In certain implementations, a pharmaceutical composition for injection is not for intravenous injection. In certain implementations, an active ingredient in a pharmaceutical composition for injection includes a compound having a structure according to Formula A. In certain implementations, an active ingredient in a pharmaceutical composition for injection includes a compound having a structure according to Formula B. In certain instances, an active ingredient in a pharmaceutical composition for injection includes a rimiducid analog, such as a compound having a structure according to Formula C, D, E, F or G, or a pharmaceutically acceptable salt, ester or amide thereof.

In a pharmaceutical composition for injection, a compound having a structure according to Formula A (a rimiducid family compound), or a compound having a structure according to Formula B (rimiducid), or a rimiducid analog compound (e.g., a compound having a structure of Formula C, D, E, F or G or a pharmaceutically acceptable salt, ester or amide thereof), sometimes is in a weight to weight (wt/wt) amount (defined herein) of about 5 mg/g to about 200 mg/g (e.g., a weight to weight (wt/wt) of amount of about 10 mg/g or greater, about 15 mg/g or greater, about 20 mg/g or greater, about 25 mg/g or greater, about 30 mg/g or greater, about 35 mg/g or greater, about 40 mg/g or greater, about 45 mg/g or greater, about 50 mg/g or greater, about 55 mg/g or greater, about 60 mg/g or greater, about 65 mg/g or greater, about 70 mg/g or greater, about 75 mg/g or greater, about 80 mg/g or greater, about 85 mg/g or greater, about 90 mg/g or greater, about 95 mg/g or greater, about 100 mg/g or greater, about 105 mg/g or greater, about 110 mg/g or greater, about 115 mg/g or greater, about 120 mg/g or greater, about 125 mg/g or greater, about 130 mg/g or greater, about 140 mg/g or greater, about 150 mg/g or greater, about 160 mg/g or greater, about 170 mg/g or greater, about 180 mg/g or greater, or about 190 mg/g or greater). For a compound having a structure according to Formula A or Formula B, a concentration of 8 mg/mL is about 5.67 millimolar (mM), a concentration of 10 mg/mL is about 7.09 mM, a concentration of 12 mg/mL is about 8.50 mM and a concentration of 20 mg/mL is about 14.2 mM, for example. In a pharmaceutical composition for injection, a compound having a structure according to Formula A (a rimiducid family compound), or a compound having a structure according to Formula B (rimiducid), or a rimiducid analog compound (e.g., a compound having a structure of Formula C, D, E, F or G, or a pharmaceutically acceptable salt, ester or amide thereof) sometimes is at a concentration of about 1 millimolar (mM) to about 30 mM, or about 10 mM to about 20 mM, or about 12 mM to about 16 mM, or about 13 mM to about 15 mM (e.g., a concentration of about 14.1 mM, about 14.2 mM or 14.3 mM), or about 5 mM to about 10 mM, or about 6 mM to about 8 mM (e.g., a concentration of about 7.0 mM, 7.09 mM, 7.1 mM or 7.2 mM); or in an amount of 10 mM or greater, 15 mM or greater, 20 mM or greater, 25 mM or greater, 30 mM or greater, 35 mM or greater, 40 mM or greater, 45 mM or greater, 50 mM or greater, 55 mM or greater, 60 mM or greater, 65 mM or greater, 70 mM or greater, 75 mM or greater, 80 mM or greater, 85 mM or greater, 90 mM or greater, 95 mM or greater, 100 mM or greater, 105 mM or greater, 110 mM or greater, 115 mM or greater, 120 mM or greater, 125 mM or greater, or 130 mM or greater. In certain implementations, a total volume of a pharmaceutical composition for injection is about 0.1 mL to about 10 mL, or about 0.5 mL to about 5 mL, or about 1 mL to about 5 mL, or about 1 mL to about 3 mL, or about 2 mL.

In certain implementations, a pharmaceutical composition for injection can include an active ingredient and an oil component. In certain instances, a pharmaceutical composition for injection includes an active ingredient, an oil component, no aqueous component, and optionally a surfactant component.

In certain implementations, a pharmaceutical composition for injection includes an active ingredient, an aqueous component, a solvent component and a surfactant component. In certain implementations, a pharmaceutical composition for injection includes an active ingredient, an aqueous component, a surfactant component and an oil component. In certain implementations, a pharmaceutical composition for injection includes an active ingredient, an oil component and a solvent component (e.g., polar solvent component). In certain implementations, a pharmaceutical composition for injection includes an active ingredient and a solvent component (e.g., polar solvent component). In certain implementations, a pharmaceutical composition for injection includes an active ingredient, an aqueous component, a solvent component, a surfactant component and an oil component.

### Oil Component

An oil component in a pharmaceutical composition for injection can include one or more of medium-chain triglycerides (MCT), castor oil (CO), medium-chain triglycerides of caprylic (C8) and capric (C10) acids (CAP) (e.g., Labrafac®), oleoyl polyoxyl-6 glycerides (OPG) (e.g., Labrafil®, Labrafil® M 1944 CS), polyethoxylated castor oil (PECO) (e.g., Cremophor EL®), a triglyceride having a structure according to the formula C₃H₅(OCOCH₃)₃ (i.e., Triacetin), oleic acid (OA), propylene glycol monocaprylate (PGMC) (Type II) (e.g., Capryol®90), glycerol monooleate (GMO) (e.g., Peceol®, Span40), caprylocaproyl polyoxyl-8 glycerides (CCPG) (Labrasol®), propylene glycol monolaurate (PGML) Type II (e.g., Lauroglycol™ 90), glyceryl monolinoleate (GML) (e.g., Maisine® CC) and Pluronic® polymers (PP). A Pluronic® polymers (PP) additive can include synthetic triblock copolymers containing hydrophobic polypropylene oxide (PPO) and hydrophilic polyethylene oxide (PEO) arranged in a PEO-PPO-PEO manner. An amount of an oil component by volume in a pharmaceutical composition for injection, prepared as an emulsion, can be about 5% to about 50%, or about 10% to about 40%, or about 20% to about 40%, or about 25% to about 35% or about 30% of the oil compound in the pharmaceutical composition. An amount of an oil component by volume in a pharmaceutical composition for injection, prepared as an emulsion, can be about 5% to about 50%, or about 10% to about 40%, or about 20% to about 40%, or about 25% to about 35% or about 30%, or in another amount described herein.

An oil component in a pharmaceutical composition for injection can include a mixture of oils, such as two or more, three or more, four or more or five or more oil components chosen from MCT, CO, CAP, OPG, PECO, Triacetin, OA, PGMC, GMO, CCPG, PGML, GML and PP. For an oil component containing two more components, a first component and a second component can be included at a ratio between 95:5 and 5:95 (e.g., about 10:90, 15:85, 20:80, 25:75, 30:70, 35:65, 40:60, 45:55, 50:50, 55:45, 60:40, 65:35, 70:30, 75:25, 80:20, 85:15 and 90:10), which ratio may be weight to weight or volume to volume. In certain implementations, an oil component includes MCT:CO at a ratio of about 60:40 to about 40:60 or about 50:50, by weight (w:w). In certain implementations, an oil component includes Triacetin:PECO at a ratio of about 30:70 to about 50:50 or about 33:67, by weight (w:w). In certain implementations, an oil component of a pharmaceutical composition for injection consists of or consists essentially of a single oil (e.g., MCT, CO, CAP, OPG, PECO, Triacetin, OA, PGMC, GMO, CCPG, PGML, GML or PP).

In certain instances, an oil component of a pharmaceutical composition for injection includes a vegetable oil or a vegetable oil derivative. Non-limiting examples of vegetable oils include cottonseed oil, castor oil (CO), sesame oil, almond oil, canola oil, corn oil, coconut oil, mineral oil, olive oil, soybean oil, sunflower oil and safflower oil. Non-limiting examples of vegetable oil derivatives include hydrogenated corn oil and PECO. An amount by volume of an oil component containing a vegetable oil or a vegetable oil derivative in a pharmaceutical composition for injection, prepared as a predominantly oil composition, can be about 50% to about 100%, about 60% to about 100%, or about 70% to about 100%, or about 80% to about 100%, or about 85% to about 100%, or about 90% to about 100%, or about 95% to about 100% of the oil component. An amount of vegetable oil or vegetable oil derivative by volume in a pharmaceutical composition for injection, prepared as an emulsion composition, can be about 5% to about 50%, or about 10% to about 40%, or about 20% to about 40%, or about 25% to about 35% or about 30% of the oil component.

In certain implementations, an oil component of a pharmaceutical composition for injection includes MCT, consists of MCT or consists essentially of MCT. An oil component in a pharmaceutical composition consisting essentially of MCT can include one or more other components that do not materially affect the basic and novel characteristics of the MCT. A component that does not materially affect the basic and novel characteristics of the MCT typically alters the solubility of an active ingredient (e.g., rimiducid, rimiducid family compound or rimiducid analog) in the pharmaceutical composition by 5% or less. MCT typically contains 95% or more caprylic/capric triglyceride (saturated), and the purity of the MCT oil typically is at least 90% (e.g., at least 95%; about 100%; USP grade). An amount of MCT by volume in a pharmaceutical composition for injection, prepared as an emulsion (described herein), can be about 5% to about 50%, or about 10% to about 40%, or about 20% to about 40%, or about 25% to about 35% or about 30%. A pharmaceutical composition for injection, prepared as an emulsion, can include about 110 mM to about 1170 mM MCT, or about 200 mM to about 1000 mM MCT, or about 300 mM to about 1000 mM MCT, or about 400 mM to about 1000 mM MCT, or about 500 mM to about 900 mM MCT, or about 600 mM to about 800 mM MCT, or about 650 mM to about 750 mM MCT, or about 690 mM to about 700 mM MCT, or about 695 mM to about 700 mM MCT (e.g., about 696 mM MCT, about 697 mM MCT, about 698 mM MCT, about 699 mM MCT). An amount of MCT by volume in a pharmaceutical composition for injection, prepared predominantly as an oil (described herein), can be about 50% to about 100%, about 60% to about 100%, or about 70% to about 100%, or about 80% to about 100%, or about 85% to about 100%, or about 90% to about 100%, or about 95% to about 100%. A pharmaceutical composition for injection, prepared predominantly as an oil, can include about 1170 mM to about 2323 mM MCT, or about 1300 mM to about 2323 mM MCT, or about 1500 mM to about 2323 mM MCT, or about 1700 mM to about 2323 mM MCT, or about 1900 mM to about 2323 mM MCT, or about 2000 mM to about 2323 mM MCT, or about 2100 mM to about 2323 mM MCT, or about 2200 mM to about 2323 mM MCT (e.g., about 2250 mM MCT, about 2300 mM MCT).

In certain instances, an oil component of a pharmaceutical composition for injection includes CO, consists of CO or consists essentially of CO. An oil component in a pharmaceutical composition for injection consisting essentially of CO can include one or more other components that do not materially affect the basic and novel characteristics of the CO. A component that does not materially affect the basic and novel characteristics of the CO typically alters the solubility of an active ingredient (e.g., rimiducid, rimiducid family compound or rimiducid analog) in the pharmaceutical composition by 5% or less. A CO component of a pharmaceutical composition for injection often contains triglycerides of fatty acids, with a fatty acid composition sometimes of about 87% ricinoleic acid, about 7% oleic acid, about 3% linoleic acid, about 2% palmitic acid, about 1% stearic acid, and trace amounts of dihydroxystearic acid. An amount of CO by volume in a pharmaceutical composition for injection, prepared as an emulsion, can be about 5% to about 50%, or about 10% to about 40%, or about 20% to about 40%, or about 25% to about 35% or about 30%. A pharmaceutical composition for injection, prepared as an emulsion, can include about 50 mM to about 520 mM CO, or about 100 mM to about 450 mM CO, or about 150 mM to about 400 mM CO, or about 200 mM to about 400 mM CO, or about 250 mM to about 350 mM CO, or about 280 mM to about 320 mM CO, or about 300 mM to about 310 mM CO, or about 305 mM to about 310 mM CO (e.g., about 306 mM CO, about 307 mM CO, about 308 mM CO, about 309 mM CO). An amount of CO by volume in a pharmaceutical composition for injection, prepared as a predominantly oil composition, can be about 50% to about 100%, about 60% to about 100%, or about 70% to about 100%, or about 80% to about 100%, or about 85% to about 100%, or about 90% to about 100%, or about 95% to about 100%. A pharmaceutical composition for injection, prepared as a predominantly oil composition, can include about 600 mM to about 1023 mM CO, or about 700 mM to about 1023 mM CO, or about 800 mM to about 1023 mM CO, or about 900 mM to about 1023 mM CO, or about 950 mM to about 1023 mM CO, or about 1000 mM to about 1023 mM CO.

### Aqueous Component

A pharmaceutical composition sometimes includes an aqueous component, such as water or saline, for example. In certain implementations, an aqueous component consists of water or consists essentially of water. An aqueous component in a pharmaceutical composition consisting essentially of water can include one or more other components that do not materially affect the basic and novel characteristics of the water. A component that does not materially affect the basic and novel characteristics of the water typically alters the solubility of an active ingredient (e.g., rimiducid, rimiducid family compound or rimiducid analog) in the pharmaceutical composition by 5% or less.

### Emulsions

A pharmaceutical composition for injection that includes an oil component and aqueous component can be an oil-in-water emulsion containing particles (e.g. a nano-emulsion). An oil-in-water emulsion often contains a surfactant component, which can form micelles in the pharmaceutical composition. A surfactant component can include one or more surfactants described herein, such as PS80, for example.

In certain instances the particles are defined by a mean particle diameter of about 1 nanometer (nm) to about 2000 nm. An emulsion sometimes is characterized by a mean particle diameter of 200 nm or less, or 150 nm or less, or 100 nm or less, or 75 nm or less, or 50 nm or less. In certain instances, a standard deviation of the mean particle diameter is about 30 nm or less, or about 25 nm or less, or about 20 nm or less, or about 15 nm or less, or about 10 nm or less, or about 5 nm or less, or about 1 nm or less, or about 0.5 nm or less, or about 0.01 nm or less. Particles in an emulsion sometimes are defined by dispersity. Dispersity is the standard deviation (a) of the particle diameter distribution divided by the mean particle diameter and is alternatively referred to herein as polydispersity. In certain instances, the particles are defined by a dispersity of about 0.005 to about 0.50, or about 0.01 to about 0.40, or about 0.01 to about 0.30, or about 0.01 to about 0.25, or about 0.10 to about 0.25. In certain instances, a standard deviation of the dispersity is about 0.25 or less, about 0.20 or less, about 0.15 or less, about 0.10 or less, about 0.05 or less, about 0.01 or less, about 0.005 or less or about 0.001 or less. Particle size and dispersity can be determined using a suitable known technique, such as Dynamic Light Scattering (DLS), for example. Devices for DLS determination are commercially available (e.g., Zetasizer device (Malvern Panalytical Ltd., Malvern, United Kingdom).

In certain implementations, the particles in an emulsion are defined by a mean particle diameter of about 100 nm to about 200 nm, or about 130 nm to about 170 nm, or about 140 nm to about 160 nm, or about 150 nm (e.g., about 148.6 nm). In certain instances, a standard deviation of the mean particle diameter is about 0.5 nm to about 5 nm, or about 0.5 nm to about 4 nm, or about 1 nm to about 3 nm or about 2 nm (e.g., about 1.96). In certain instances, the particles are defined by a dispersity of about 0.05 to about 0.30, or about 0.10 to about 0.20, or about 0.12. In certain instances, a standard deviation of the dispersity is about 0.005 to about 0.10, or about 0.01 to about 0.03, or about 0.02.

In certain implementations, the particles in an emulsion are defined by a mean particle diameter of about 100 nm to about 300 nm, or about 180 nm to about 190 nm, or about 185 nm (e.g., about 185.03 nm). In certain instances, a standard deviation of the mean particle diameter is about 0.5 nm to about 10 nm, or about 2 nm to about 4 nm, or about 3 nm (e.g., about 2.85). In certain instances, the particles are defined by a dispersity of about 0.005 to about 0.10, or about 0.01 to about 0.03, or about 0.02. In certain instances, a standard deviation of the dispersity is about 0.005 to about 0.10, or about 0.01 to about 0.03, or about 0.02.

In certain implementations, the particles in an emulsion are defined by a mean particle diameter of about 500 nm to about 800 nm, or about 600 nm to about 750 nm or about 670 nm (e.g., about 668.37 nm). In certain instances, a standard deviation of the mean particle diameter is about 15 nm to about 35 nm, or about 20 nm to about 25 nm, or about 22 nm to about 23 nm (e.g., about 22.47 nm). In certain instances, the particles are defined by a dispersity of about 0.2 to about 0.65, or about 0.4 to about 0.5, or about 0.44. In certain instances, a standard deviation of the dispersity is about 0.2 to about 0.65, or about 0.4 to about 0.5, or about 0.42.

In certain implementations, the particles in an emulsion are defined by a mean particle diameter of about 1000 nm to about 1400 nm, or about 1100 nm to about 1300 nm, or about 1200 nm to about 1250 nm, or about 1228 nm to about 1229 nm, or about 1228 nm (e.g., about 1228.67 nm). In certain instances, a standard deviation of the mean particle diameter is about 1 nm to about 20 nm, or about 8 nm to about 12 nm, or about 9 nm to about 11 nm, or about 10 nm (e.g., 10.12 nm). In certain instances, the particles are defined by a dispersity of about 0.8 to about 1.2, or about 0.95 to about 1.05, or about 1.0. In certain instances, a standard deviation of the dispersity is about 0 to about 0.1, or about 0 to about 0.01, or about 0.

### Surfactant Component

A pharmaceutical composition for injection can include a surfactant component. A surfactant component sometimes is a solubility enhancer component. Non-limiting examples of a solubility enhancer component include lecithin; polysorbate (PS) 20, 40, 60 and/or 80; Span 20, 40, 60 and/or 80; Pluronic® polymers (PP); propylene glycol (PG) and cyclodextrin (CD). A PS additive can include a polyoxyethylene (20) sorbitan mono-ester molecule, with “(20)” referring to the total number of oxyethylene groups, including without limitation polyoxyethylene (20) sorbitan monolaurate (PS20), polyoxyethylene (20) sorbitan monopalmitate (PS40), polyoxyethylene (20) sorbitan monostearate (PS60), and/or polyoxyethylene (20) sorbitan monooleate (PS80). Polysorbate 80 (PS80) also is characterized as a polyoxyethylene sorbitan fatty acid ester with a molecular weight of 1310 Da. A “Span” additive can include sorbitan monolaurate (Span20), sorbitan monopalmitate (Span40), sorbitan monostearate (Span60), and/or sorbitan monooleate (Span80). A Pluronic® polymers (PP) additive is described herein. A cyclodextrin (CD) additive can include one or more cyclodextrin types, where each cyclodextrin type is a macrocyclic ring of glucose subunits joined by alpha-1,4 glycosidic bonds. Non-limiting examples of CD types include alpha-CD that includes 6 glucose units, beta-CD that includes 7 glucose units and gamma-CD that includes 8 glucose units.

In certain implementations, a surfactant component or solubility enhancer component in a pharmaceutical composition for injection includes a polysorbate, or consists of a polysorbate or consists essentially of a polysorbate. In certain implementations, a surfactant component or solubility enhancer component in a pharmaceutical composition for injection includes PS80, or consists of PS80 or consists essentially of PS80. A surfactant component or solubility enhancer component in a pharmaceutical composition for injection consisting essentially of a polysorbate (e.g., PS80), can include one or more other components that do not materially affect the basic and novel characteristics of the polysorbate (e.g., PS80). A component that does not materially affect the basic and novel characteristics of the polysorbate (e.g., PS80) typically alters the solubility of an active ingredient (e.g., rimiducid, rimiducid family compound or rimiducid analog) in the pharmaceutical composition by 5% or less. A pharmaceutical composition for injection that contains a polysorbate, such as PS80, sometimes includes the polysorbate (e.g., PS80) at a percent volume of about 0.1% to about 35%, or about 1% to about 40%, or about 5% to about 35%, or about 10% to about 30%, or about 15% to about 30%, or about 15% to about 25%, or about 10%, or about 15%, or about 20%, or about 25% or about 30%. In a pharmaceutical composition for injection containing PS80, 20% by volume of PS80 is about 162 mM PS80, 30% by volume of PS80 is about 243 mM PS80 and 40% by volume of PS80 is about 323 mM PS80. In certain implementations, a pharmaceutical composition for injection contains about 5 mM to about 330 mM PS80, or about 40 mM to about 290 mM PS80, about 80 mM to about 250 mM PS80, or about 120 mM to about 240 mM PS80, or about 120 mM to about 200 mM PS80, or about 60 mM to about 100 mM PS80, or about 70 mM to about 90 mM PS80, or about 75 mM to about 85 mM PS80, or about 80 mM PS80, or about 110 mM to about 130 mM PS80, or about 115 mM to about 125 mM PS80, or about 120 mM PS80, or about 150 mM to about 170 mM PS80, or about 155 mM to about 165 mM PS80, or about 160 mM PS80, or about 220 mM to about 260 mM PS80, or about 230 mM to about 250 mM PS80, or about 235 mM to about 245 PS80, or about 238 mM to about 242 mM PS80 (e.g., about 239 mM PS80, about 240 mM PS80, about 241 mM PS80) in the pharmaceutical composition.

### Penetration Enhancer Component

A pharmaceutical composition for injection can include a penetration enhancer component. Non-limiting examples of a penetration enhancer component include ethanol, NMP and 2-(2-ethoxyethoxy)ethanol (EEE), having formula CH₃CH₂OCH₂CH₂OCH₂CH₂OH), which is commercially available under the tradename Transcutol® and Transcutol® HP.

### Solvent Component

A pharmaceutical composition for injection sometimes includes a solvent component. In certain implementations, a pharmaceutical composition for injection includes a solvent component containing one or more polar solvents. A polar solvent sometimes includes a polyethylene glycol (PEG), consists of a PEG, or consists essentially of a PEG. Non-limiting examples of polar solvents are N-methyl 2-pyrrolidone (NMP), polyethylene glycol 300 (PEG300), polyethylene glycol 400 (PEG400), dimethyl sulfoxide (DMSO), 2-(2-ethoxyethoxy)ethanol (EEE) or ethanol. A pharmaceutical composition for injection sometimes is a predominantly polar solvent composition. An amount by volume of a polar solvent component containing one or more polar solvents, in a predominantly polar solvent pharmaceutical composition, can be about 50% to about 100%, about 60% to about 100%, or about 70% to about 100%, or about 80% to about 100%, or about 85% to about 100%, or about 90% to about 100%, or about 95% to about 100%.

In certain instances, a solvent component in a pharmaceutical composition containing such a component includes PEG400, consists of PEG400, or consists essentially of PEG400. A solvent component in a pharmaceutical composition consisting essentially of a PEG (e.g., PEG400), can include one or more other components that do not materially affect the basic and novel characteristics of the PEG (e.g., PEG400). A component that does not materially affect the basic and novel characteristics of the PEG (e.g., PEG400) typically alters the solubility of an active ingredient (e.g., rimiducid, rimiducid family compound or rimiducid analog) in the pharmaceutical composition by 5% or less. Pharmaceutical compositions provided herein that include PEG400 sometimes include about 1% to about 40% PEG400, or about 20% to about 40% PEG400, or about 5% to about 25% PEG400, or about 10% to about 20% PEG400, or about 30% PEG400, or about 15% PEG400. In a pharmaceutical composition for injection containing PEG400, PEG400 at a percent by volume of 20% is about 565 mM PEG400, PEG400 at a percent by volume of 30% is about 848 mM PEG400, and PEG400 at a percent by volume of 40% is about 1130 mM. A pharmaceutical composition for injection sometimes includes about 550 mM to about 1150 mM PEG400, or about 650 mM to about 1000 mM PEG400, or about 750 mM to about 900 mM PEG400, or about 800 mM to about 900 mM PEG400, or about 840 mM to about 860 mM PEG400, or about 845 mM to about 855 mM PEG400, or about 846 mM to about 850 mM PEG400.

### Particular Pharmaceutical Compositions for Administration by Injection

In certain instances, a pharmaceutical composition includes particular combinations of an oil component, aqueous component and one or more of a solubility enhancer component and a penetration enhancer component. In a hypothetical pharmaceutical composition that includes three additives x, y and z, component x may be at a weight percent of about x″ that is between a weight percent of about x′ to about x″, component y may be at a weight percent of about y′″ that is between a weight percent of about y′ to about y″, and component z may be at a weight percent of about z′″ that is between a weight percent of about z′ to about z″, where the weight percent of component x, y or z is relative to total weight of other additives in the pharmaceutical composition. As used herein, such ratios of components x, y and z can be expressed as “x′-x″:y′-y″:z′-z” and can be expressed as “x′″:y′″:z″” which are provided according to weight percentage (i.e., wt %).

In certain implementations, additives in a pharmaceutical composition include MCT:water:PS80:Span80:ethanol (25-35:45-55:5-10:5-10:1-10), or sometimes include MCT:water:PS80:Span80:ethanol (30:50:7.5:7.5:5). In certain instances, additives in a pharmaceutical composition include CO:saline:PS60:EEE (15-25:60-70:5-15:1-10), or sometimes include CO:saline:PS60:EEE (20:65:10:5). In certain instances, additives in a pharmaceutical composition include CO:saline:PS80 (10-20:65-75:10-20), or sometimes include CO:saline:PS80 (15:70:15). In certain instances, additives in a pharmaceutical composition include OA:saline:Span40:EEE (30-40:45-55:5-15:1-10), or sometimes include OA:saline:Span40:EEE (35:50:10:5). In certain instances, additives in a pharmaceutical composition include PECO:saline:Span80 (10-20:70-80:5-15), or sometimes include PECO:saline:Span80 (15:75:10).

In certain instances, additives in a pharmaceutical composition include CAP:saline:lecithin (15-25:45-55:25-35), or sometimes include CAP:saline:lecithin (20:50:30). In certain instances, additives in a pharmaceutical composition include OPG:saline:CD:EEE (10-20:60-70:10-20:1-10), or sometimes include OPG:saline:CD:EEE (15:65:15:5). In certain instances, additives in a pharmaceutical composition include PGMC:saline:PP:ethanol (25-35:45-55:10-20:1-10), or sometimes include PGMC:saline:PP:ethanol (30:50:15:5).

In certain implementations, a pharmaceutical composition includes CO (5-30 wt %), saline (50-75 wt %), PS80 (15-30 wt %) and rimiducid (2.5-10 wt %), which sometimes is in dosage form and sometimes is for subcutaneous administration. The term “wt %” as used in the foregoing sentence is a percentage of the weight of a specified component to the total weight of active ingredient(s) and additives in the pharmaceutical composition.

In certain implementations, an active ingredient in a pharmaceutical composition consists of or consists essentially of a compound having a structure according to Formula A (rimiducid family compound) or a compound having a structure according to Formula B (rimiducid). An active ingredient in a pharmaceutical composition consisting essentially of a compound having a structure according to Formula A or Formula B can include one or more other components that do not materially affect the basic and novel characteristics of a compound having a structure according to Formula B. A component that does not materially affect the basic and novel characteristics of a compound having a structure according to Formula B typically alters the solubility of the compound having the structure according to Formula B in the pharmaceutical composition by 5% or less.

### Predominantly Oil Pharmaceutical Compositions for Injection

In certain implementations, a pharmaceutical composition for injection can include an active ingredient and an oil component. In certain instances, a pharmaceutical composition contains (i) no aqueous component; (ii) contains no water; (iii) contains no added water; (iv) contains no surfactant component; (v) contains no solvent component; (vi) contains no polar solvent component; or (vii) a combination of two, three, four, five or all of (i), (ii), (iii), (iv), (v) or (vi). In certain instances, a pharmaceutical composition for injection consists of or consists essentially of an active ingredient and an additive consisting of or consisting essentially of an oil component. In certain instances, a pharmaceutical composition for injection includes an active ingredient, an oil component, no aqueous component, and optionally a surfactant component. In certain instances, a pharmaceutical composition for injection includes an active ingredient, an oil component, and optionally a solvent component containing one or more polar solvents. A pharmaceutical composition for injection sometimes includes one or more antioxidants or other preservatives.

An active ingredient in a predominantly oil pharmaceutical composition for injection often includes, consists of, or consists essentially of a compound having a structure according to Formula A, a compound having a structure according to Formula B, a rimiducid analog (e.g., a compound having a structure according to Formula C, D, E, F or G, or a pharmaceutically acceptable salt, ester or amide thereof). In certain implementations, the active ingredient is at a concentration in the pharmaceutical composition of about 1 milligram per milliliter (mg/mL) to about 40 mg/mL, or about 5 milligrams per milliliter (mg/mL) to about 30 mg/mL, or about 10 mg/mL to about 25 mg/mL, or about 15 mg/mL to about 25 mg/mL, or about 19 mg/mL to about 21 mg/mL (e.g., about 20 mg/mL), about 5 mg/mL to about 15 mg/mL, or about 8 mg/mL to about 12 mg/mL, or about 9 mg/mL to about 11 mg/mL (e.g., about 10 mg/mL). In certain instances, the active ingredient is at a concentration in the pharmaceutical composition of about 1 millimolar (mM) to about 25 mM, or about 5 mM to about 20 mM, or about 10 mM to about 20 mM, or about 12 mM to about 16 mM, or about 13 mM to about 15 mM (e.g., a concentration of about 14.1 mM, 14.2 mM or 14.3 mM), or about 5 mM to about 10 mM, or about 6 mM to about 9 mM, or about 6 mM to about 8 mM (e.g., about 7.0 mM, 7.09 mM, 7.1 mM or about 7.2 mM). In certain implementations, a compound having a structure according to Formula A or Formula B or a rimiducid analog is present in a predominantly oil pharmaceutical composition described herein in a weight to weight (wt/wt) amount of about 5 mg/g to about 200 mg/g (e.g., about 10 mg/g or greater, about 15 mg/g or greater, about 20 mg/g or greater, about 25 mg/g or greater, about 30 mg/g or greater, about 35 mg/g or greater, about 40 mg/g or greater, about 45 mg/g or greater, about 50 mg/g or greater, about 55 mg/g or greater, about 60 mg/g or greater, about 65 mg/g or greater, about 70 mg/g or greater, about 75 mg/g or greater, about 80 mg/g or greater, about 85 mg/g or greater, about 90 mg/g or greater, about 95 mg/g or greater, about 100 mg/g or greater, about 105 mg/g or greater, about 110 mg/g or greater, about 115 mg/g or greater, about 120 mg/g or greater, about 125 mg/g or greater, about 130 mg/g or greater, about 140 mg/g or greater, about 150 mg/g or greater, about 160 mg/g or greater, about 170 mg/g or greater, about 180 mg/g or greater, or about 190 mg/g or greater). In certain implementations, a compound having a structure according to Formula A or Formula B or a rimiducid analog is present in a predominantly oil pharmaceutical composition described herein in a concentration of about 5 millimolar (mM) to about 140 mM (e.g., about 10 mM or greater, about 15 mM or greater, about 20 mM or greater, about 25 mM or greater, about 30 mM or greater, about 35 mM or greater, about 40 mM or greater, about 45 mM or greater, about 50 mM or greater, about 55 mM or greater, about 60 mM or greater, about 65 mM or greater, about 70 mM or greater, about 75 mM or greater, about 80 mM or greater, about 85 mM or greater, about 90 mM or greater, about 95 mM or greater, about 100 mM or greater, about 105 mM or greater, about 110 mM or greater, about 115 mM or greater, about 120 mM or greater, about 125 mM or greater, or about 130 mM or greater). In certain implementations, a total volume of a pharmaceutical composition is about 0.1 mL to about 10 mL, or about 0.5 mL to about 5 mL, or about 1 mL to about 5 mL, or about 1 mL to about 3 mL, or about 2 mL.

The amount of an oil component in a predominantly oil pharmaceutical composition for injection can be about 50% to about 100%, about 60% to about 100%, or about 70% to about 100%, or about 80% to about 100%, or about 85% to about 100%, or about 90% to about 100%, or about 95% to about 100%, by volume in the pharmaceutical composition. A percent volume amount of an oil component in a predominantly oil pharmaceutical composition for injection can be about 50% or greater, or about 60% or greater, or about 70% or greater, or about 80% or greater, or about 85% or greater, or about 90% or greater, or about 95% or greater, or about 99% or greater. An oil in a predominantly oil pharmaceutical composition often is at least 90% pure, and sometimes is at least 95% pure. An oil in a predominantly oil pharmaceutical composition can be any suitable oil, such as an oil described herein, and sometimes includes MCT, CO, PECO (e.g., Cremophor EL®) or cottonseed oil.

An oil component in a predominantly oil pharmaceutical composition for injection sometimes consists of or consists essentially of MCT. The amount of MCT in a predominantly oil pharmaceutical composition for injection can be about 50% or greater, or about 60% or greater, or about 70% or greater, or about 80% or greater, or about 85% or greater, or about 90% or greater, or about 95% or greater, or about 99% or greater, MCT. The amount of MCT in a predominantly oil pharmaceutical composition for injection can be about 1170 mM to about 2323 mM MCT, or about 1300 mM to about 2323 mM MCT, or about 1500 mM to about 2323 mM MCT, or about 1700 mM to about 2323 mM MCT, or about 1900 mM to about 2323 mM MCT, or about 2000 mM to about 2323 mM MCT, or about 2100 mM to about 2323 mM MCT, or about 2200 mM to about 2323 mM MCT (e.g., about 2250 mM, about 2300 mM) in the pharmaceutical composition. An oil component in a predominantly oil pharmaceutical composition for injection sometimes consists of or consists essentially of CO. The amount of CO in a predominantly oil pharmaceutical composition for injection can be about 50% or greater, or about 60% or greater, or about 70% or greater, or about 80% or greater, or about 85% or greater, or about 90% or greater, or about 95% or greater, or about 99% or greater, CO. The amount of CO in a predominantly oil pharmaceutical composition for injection can be about 600 mM to about 1023 mM CO, or about 700 mM to about 1023 mM CO, or about 800 mM to about 1023 mM CO, or about 900 mM to about 1023 mM CO, or about 950 mM to about 1023 mM CO, or about 1000 mM to about 1023 mM CO, in the pharmaceutical composition.

The amount of a surfactant component, if present in a predominantly oil pharmaceutical composition for injection, can be about 0.1% to about 35%, or about 1% to about 40%, or about 5% to about 35%, or about 10% to about 30%, or about 15% to about 30%, or about 15% to about 25%, or about 10%, or about 15%, or about 20%, or about 25% or about 30%, by volume in the pharmaceutical composition. A surfactant component, if present in a predominantly oil pharmaceutical composition for injection, sometimes consists of or consists essentially of PS80. A predominantly oil pharmaceutical composition for injection sometimes contains about 5 mM to about 330 mM PS80, or about 40 mM to about 290 mM PS80, about 80 mM to about 250 mM PS80, or about 120 mM to about 240 mM PS80, or about 120 mM to about 200 mM PS80, or about 60 mM to about 100 mM PS80, or about 70 mM to about 90 mM PS80, or about 75 mM to about 85 mM PS80, or about 80 mM PS80, or about 110 mM to about 130 mM PS80, or about 115 mM to about 125 mM PS80, or about 120 mM PS80, or about 150 mM to about 170 mM PS80, or about 155 mM to about 165 mM PS80, or about 160 mM PS80, or about 220 mM to about 260 mM PS80, or about 230 mM to about 250 mM PS80, or about 235 mM to about 245 PS80, or about 238 mM to about 242 mM PS80 (e.g., about 239 mM PS80, about 240 mM PS80, about 241 mM PS80) in the pharmaceutical composition.

In certain implementations, a predominantly oil pharmaceutical composition for injection includes about 45% by volume or less of other components, including as an aqueous component (e.g., water), or a solvent component (e.g., a polar solvent component containing one or more of NMP, PEG300, PEG400, DMSO, EEE or ethanol), or surfactant component (e.g., PS80), or combination of two or more of an aqueous component, solvent component or surfactant component, for example. In certain instances, a predominantly oil pharmaceutical composition for injection includes about 40% by volume or less, about 35% by volume or less, about 30% by volume or less, about 25% by volume or less, about 20% by volume or less, about 15% by volume or less, about 10% by volume or less, about 9% by volume or less, about 8% by volume or less, about 7% by volume or less, about 6% by volume or less, about 5% by volume or less, about 4% by volume or less, about 3% by volume or less, about 2% by volume or less, about 1% by volume or less of other components.

In certain implementations, a predominantly oil pharmaceutical composition for injection contains an active ingredient (e.g., a compound having a structure according to Formula A, a compound having a structure according to Formula B, or a rimiducid analog), an oil component and a solvent component. In certain instances, a solvent component includes one or more polar solvents and is referred to as a polar solvent component. In certain implementations, a polar solvent component includes one or more of NMP, PEG300, PEG400, DMSO, EEE or ethanol. The EEE sometimes is Transcutol®. In a predominantly oil pharmaceutical composition for injection containing an oil component and polar solvent component, the oil component sometimes includes polyethoxylated castor oil (PECO), castor oil or cottonseed oil, and the PECO sometimes is Cremophor EL®. In a predominantly oil pharmaceutical composition for injection containing an oil component and polar solvent component, the active ingredient often is at least 90% pure and sometimes at least 95% pure, the oil component often is at least 90% pure and sometimes at least 95% pure, and the one or more of the polar solvents often are at least 90% pure and sometimes at least 95% pure. In certain implementations, a predominantly oil pharmaceutical composition containing a solvent component contains an amount by volume of the solvent component of about 45% (v/v) or less (e.g., about 40% (v/v) or less, about 35% (v/v) or less, about 25% (v/v) or less, about 20% (v/v) or less, about 15% (v/v) or less, about 10% (v/v) or less or about 5% (v/v) or less of a solvent component). In certain implementations, a predominantly oil pharmaceutical composition containing a solvent component contains no added water, or contains no aqueous component (e.g., saline). In certain implementations, a predominantly oil pharmaceutical composition containing a solvent component contains an aqueous component (e.g., water; saline), sometimes in an amount by volume of about 20% (v/v) or less (e.g., about 15% (v/v) or less, about 10% (v/v) or less or about 5% (v/v) or less of an aqueous component).

In certain implementations, a predominantly oil pharmaceutical composition for injection includes one polar solvent, which sometimes is NMP, PEG300, PEG400, DMSO, EEE or ethanol. The oil component and the polar solvent each are in a percent volume (v/v) amount in the pharmaceutical composition, and the sum of the percent volume of each of the oil component and the polar solvent component in the pharmaceutical composition can be about 100%. In certain instances, a predominantly oil pharmaceutical composition for injection includes about 80% to about 99% (v/v) oil component and about 1% to about 20% (v/v) PEG300. In certain instances, a predominantly oil pharmaceutical composition for injection includes about 51% to about 99% (v/v) oil component and about 1% to about 49% (v/v) PEG400. In certain instances, a predominantly oil pharmaceutical composition for injection includes about 51% to about 99% (v/v) oil component and about 1% to about 49% (v/v) NMP. In certain instances, a predominantly oil pharmaceutical composition for injection includes about 90% to about 99% (v/v) oil component and about 1% to about 10% (v/v) DMSO. In certain instances, a predominantly oil pharmaceutical composition for injection includes about 94% to about 99% (v/v) NMP and about 1% to about 6% (v/v) ethanol.

In certain implementations, a predominantly oil pharmaceutical composition for injection includes a polar solvent component containing two polar solvents. The oil component and the polar solvents in the polar solvent composition each are in a percent volume (v/v) amount in the pharmaceutical composition, and the sum of the percent volume of each of the oil component and polar solvents in the pharmaceutical composition can be about 100%. In certain instances, a predominantly oil pharmaceutical composition for injection includes about 50% to about 98% (v/v) oil component, about 1% to about 20% (v/v) PEG300 and about 1% to about 40% (v/v) NMP. In certain instances, a predominantly oil pharmaceutical composition for injection includes about 50% to about 98% (v/v) oil component, about 1% to about 20% (v/v) PEG400 and about 1% to about 40% (v/v) NMP. In certain instances, a predominantly oil pharmaceutical composition for injection includes about 50% to about 98% (v/v) oil component, about 1% to about 40% (v/v) NMP and about 1% to about 10% (v/v) DMSO. In certain instances, a predominantly oil pharmaceutical composition for injection includes about 50% to about 98% (v/v) oil component, about 1% to about 40% (v/v) NMP and about 1% to about 6% (v/v) ethanol. In certain instances, a predominantly oil pharmaceutical composition for injection includes about 50% to about 98% (v/v) oil component, about 1% to about 20% (v/v) PEG300 and about 1% to about 10% (v/v) DMSO. In certain instances, a predominantly oil pharmaceutical composition for injection includes about 50% to about 98% (v/v) oil component, about 1% to about 20% (v/v) PEG400 and about 1% to about 10% (v/v) DMSO. In certain instances, a predominantly oil pharmaceutical composition for injection includes about 50% to about 98% (v/v) oil component, about 1% to about 20% (v/v) PEG300 and about 1% to about 6% (v/v) ethanol. In certain instances, a predominantly oil pharmaceutical composition for injection includes about 50% to about 98% (v/v) oil component, about 1% to about 20% (v/v) PEG400 and about 1% to about 6% (v/v) ethanol.

In certain implementations, a predominantly oil pharmaceutical composition for injection includes a polar solvent component containing three polar solvents. The oil component and the polar solvents in the polar solvent composition each are in a percent volume (v/v) amount in the pharmaceutical composition, and the sum of the percent volume of each of the oil component and polar solvents in the pharmaceutical composition can be about 100%. In certain instances, a predominantly oil pharmaceutical composition for injection includes about 50% to about 97% (v/v) oil component, about 1% to about 40% (v/v) NMP, about 1% to about 10% (v/v) DMSO and about 1% to about 6% (v/v) ethanol. In certain instances, a predominantly oil pharmaceutical composition for injection includes about 50% to about 97% (v/v) oil component, about 1% to about 40% (v/v) NMP, about 1% to about 6% (v/v) ethanol and about 1% to about 20% (v/v) PEG300. In certain instances, a predominantly oil pharmaceutical composition for injection includes about 50% to about 97% (v/v) oil component, about 1% to about 40% (v/v) NMP, about 1% to about 6% (v/v) ethanol and about 1% to about 50% (v/v) PEG400. In certain instances, a predominantly oil pharmaceutical composition for injection includes about 50% to about 97% (v/v) oil component, about 1% to about 20% (v/v) PEG300, about 1% to about 10% (v/v) DMSO and about 1% to about 6% (v/v) ethanol. In certain instances, a predominantly oil pharmaceutical composition for injection includes about 50% to about 97% (v/v) oil component, about 1% to about 50% (v/v) PEG300, about 1% to about 10% (v/v) DMSO and about 1% to about 6% (v/v) ethanol.

In certain implementations, a predominantly oil pharmaceutical composition for injection includes a polar solvent component containing four polar solvents. The oil component and the polar solvents in the polar solvent composition each are in a percent volume (v/v) amount in the pharmaceutical composition, and the sum of the percent volume of each of the oil component and polar solvents in the pharmaceutical composition can be about 100%. In certain instances, a predominantly oil pharmaceutical composition for injection includes about 50% to about 96% (v/v) oil component, about 1% to about 20% (v/v) PEG300, about 1% to about 40% (v/v) NMP, about 1% to about 15% (v/v) DMSO and about 1% to about 6% (v/v) ethanol. In certain instances, a predominantly oil pharmaceutical composition for injection includes about 50% to about 96% (v/v) oil component, about 1% to about 20% (v/v) PEG400, about 1% to about 40% (v/v) NMP, about 1% to about 15% (v/v) DMSO and about 1% to about 6% (v/v) ethanol.

### Oil-In-Water Emulsion Pharmaceutical Compositions for Injection

In certain implementations, a pharmaceutical composition for injection includes an active ingredient, an aqueous component, a surfactant component and an oil component. In certain implementations, a pharmaceutical composition for injection includes an active ingredient, an aqueous component, a solvent component, a surfactant component and an oil component. An active ingredient in an oil-in-water emulsion pharmaceutical composition for injection often includes, consists of, or consists essentially of a compound having a structure according to Formula A, a compound having a structure according to Formula B, a rimiducid analog (e.g., a compound having a structure according to Formula C, D, E, F or G, or a pharmaceutically acceptable salt, ester or amide thereof).

### A. Non-Limiting Example of Oil-In-Water Emulsion Pharmaceutical Composition for Injection

In certain implementations, a pharmaceutical composition includes: an active ingredient including a compound having a structure according to Formula A, a compound having a structure according to Formula B; an aqueous component including water; a surfactant component including a polysorbate (PS); and an oil component including castor oil (CO). The PS sometimes includes PS80 or sometimes consists of or consists essentially of PS80. In certain implementations, the total volume of the pharmaceutical composition is about 0.1 mL to about 5 mL, or about 1 mL to about 5 mL, about 1 mL to about 3 mL, or about 2 mL.

In certain implementations, the active ingredient contains, consists of or consists essentially of a compound having a structure according to Formula A or Formula B. In certain instances, the active ingredient is at a final concentration of about 1 milligram (mg) to about 40 mg active ingredient per milliliter (mL) volume, or about 10 milligrams (mg) to about 30 mg active ingredient per mL volume, or about 15 milligrams (mg) to about 25 mg active ingredient per mL volume, or about or about 20 milligrams (mg) active ingredient per mL volume. In certain instances, a pharmaceutical composition for injection includes about 1 millimolar (mM) to about 30 mM, a concentration of about 10 mM to about 20 mM, a concentration of about 12 mM to about 16 mM, or a concentration of about 13 mM to about 15 mM (e.g., a concentration of about 14.1 mM, about 14.2 mM or 14.3 mM) of a compound having the structure according to Formula A or Formula B.

In certain implementations, a pharmaceutical composition for injection includes: an active ingredient containing, consisting of or consisting essentially of a compound having a structure according to Formula A or Formula B; an aqueous component consisting of or consisting essentially of water, a surfactant component consisting of or consisting essentially of PS80, and an oil component consisting of or consisting essentially of CO. In certain instances, the aqueous component is at a percent volume of about 50% to about 70%, the oil component is at a percent volume of about 20% to about 40%, and the surfactant component is at a percent volume of about 1% to about 20%. In certain instances, the aqueous component, the oil component and the surfactant component each are at a percent volume and the percent volumes of the aqueous component, the oil component and the surfactant component sum to 100%; the percent volume of the aqueous component is about 55% to about 65%; the percent volume of the oil component is about 25% to about 35%; and the percent volume of the surfactant component is about 5% to about 15%. In certain instances, the percent volume of the aqueous component is about 60%, the percent volume of the oil component is about 30%, and the percent volume of the surfactant component is about 10%.

In certain instances, the pharmaceutical composition for injection includes: about 8 mg to about 12 mg per mL volume, or about 15 mg to about 25 mg per mL volume, of a compound having a structure according to Formula A or Formula B; about 50% to about 70% water by volume; about 20% to about 40% CO by volume; and about 1% to about 20% PS80 by volume, where the percentage by volume of the water, where the percentage by volume of the CO and the percentage by volume of the PS80 sum to 100%. In certain instances, a pharmaceutical composition for injection includes: about 8 mg to about 12 mg per mL volume, or about 15 mg to about 25 mg per mL volume, of a compound having a structure according to Formula A or Formula B; about 55% to about 65% water by volume; about 25% to about 35% CO by volume; and about 5% to about 15% PS80 by volume, where the percentage by volume of the water; where the percentage by volume of the CO and the percentage by volume of the PS80 sum to 100%. In certain implementations, the pharmaceutical composition for injection includes: about 1 mM to about 30 mM, or about 5 mM to about 25 mM, or about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula A or Formula B; about 50 mM to about 520 mM CO, or about 100 mM to about 450 mM CO, or about 150 mM to about 400 mM CO, or about 200 mM to about 400 mM CO, or about 250 mM to about 350 mM CO, or about 280 mM to about 320 mM CO, or about 300 mM to about 310 mM CO, or about 305 mM to about 310 mM CO (e.g., about 306 mM CO, about 307 mM CO, about 308 mM CO, about 309 mM CO); about 40 mM to about 125 mM PS80, or about 60 mM to about 100 mM PS80, or about 70 mM to about 90 mM PS80, or about 75 mM to about 85 mM PS80, or about 79 mM to about 83 mM PS80 (e.g., about 80 mM PS80, about 81 mM PS80, about 82 mM PS80); and sometimes about 55% to about 65% water by volume.

In certain instances, the pharmaceutical composition includes: about 10 mg per mL volume or about 20 mg per mL volume of a compound having a structure according to Formula B; about 60% water by volume, about 30% CO by volume, and about 10% PS80 by volume. In certain implementations, the pharmaceutical composition includes: about 6.9 mM to about 7.3 mM (e.g., about 7.0 mM, about 7.1 mM, about 7.2 mM), or about 13 mM to about 15 mM (e.g., about 14.1 mM, 14.2 mM or about 14.3 mM) of a compound having the structure of Formula B; about 305 mM to about 310 mM CO (e.g., about 306 mM CO, about 307 mM CO, about 308 mM CO, about 309 mM CO); about 79 mM to about 83 mM PS80 (e.g., about 80 mM PS80, about 81 mM PS80, about 82 mM PS80); and sometimes about 60% water by volume.

The oil-in-water emulsion pharmaceutical composition for injection typically includes particles. In certain implementations, the particles have one or more of the following characteristics: (i) a mean particle diameter of 200 nm or less, or 150 nm or less, or 100 nm or less, or 75 nm or less, or 50 nm or less. In certain instances, (ii) a standard deviation of the mean particle diameter of about 30 nm or less, or about 25 nm or less, or about 20 nm or less, or about 15 nm or less, or about 10 nm or less, or about 5 nm or less, or about 1 nm or less, or about 0.5 nm or less or about 0.01 nm or less; (iii) a dispersity of about 0.005 to about 0.50, or about 0.01 to about 0.40, or about 0.01 to about 0.30, or about 0.01 to about 0.25, or about 0.10 to about 0.25; or (iv) a standard deviation of the dispersity of about 0.25 or less, about 0.20 or less, about 0.15 or less, about 0.10 or less, about 0.05 or less, about 0.01 or less, about 0.005 or less or about 0.001 or less. In certain instances, the particles have one or more of the following characteristics: (i) the particles are defined by a mean particle diameter of about 500 nm to about 800 nm, or about 600 nm to about 750 nm, or about 670 nm (e.g., about 668 nm); (ii) a standard deviation of the mean particle diameter of about 15 nm to about 35 nm, or about 20 nm to about 25 nm, or about 22.5 nm; (iii) a dispersity of about 0.2 to about 0.65, or about 0.4 to about 0.5, or about 0.44; and (iv) a standard deviation of the dispersity of about 0.2 to about 0.65 or about 0.4 to about 0.5, or about 0.42.

### B. Non-Limiting Example of Oil-In-Water Emulsion Pharmaceutical Composition for Injection

In certain implementations, a pharmaceutical composition for injection includes: an active ingredient including a compound having a structure according to Formula A, a compound having a structure according to Formula B; an aqueous component including water; a surfactant component including a polysorbate (PS); and an oil component including Medium Chain Triglycerides (MCT). The PS sometimes includes PS80 or sometimes is PS80. The MCT sometimes includes one or more of C6, C8, C10 and C12 fatty acids, and sometimes one or more or all of the C6, C8, C10 and C12 fatty acids are saturated fatty acids. The MCT sometimes includes a C8 fatty acid and a C10 fatty acid and optionally a C12 fatty acid. The MCT sometimes is from a plant, such as coconut for example.

In certain implementations, the active ingredient contains, consists of or consists essentially of a compound having a structure according to Formula A or Formula B. In certain instances, the active ingredient is at a final concentration of about 1 milligram (mg) to about 40 mg active ingredient per milliliter (mL) volume, or about 5 milligrams (g) to about 30 mg active ingredient per mL volume, or about 8 milligrams (mg) to about 12 mg active ingredient per mL volume, or about 10 milligrams (mg) active ingredient per mL volume of the pharmaceutical composition, or about 15 mg to about 25 mg active ingredient per mL volume or about 18 mg to about 22 mg active ingredient per mL volume (e.g., about 20 mg active ingredient per mL volume). In certain instances, a pharmaceutical composition for injection includes about 1 mM to about 30 mM, or about 5 mM to about 25 mM, or about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure according to Formula A or Formula B.

In certain implementations, the pharmaceutical composition includes: an active ingredient containing, consisting of or consisting essentially of a compound having a structure according to Formula A or Formula B; an aqueous component consisting of or consisting essentially of water, a surfactant component consisting of or consisting essentially of PS80, and an oil component consisting of or consisting essentially of MCT. In certain instances, the aqueous component is at a percent volume of about 50% to about 70%, the oil component at a percent volume of about 20% to about 40%, and the surfactant component at a percent volume of about 1% to about 20%. In certain instances, the aqueous component, the oil component and the surfactant component each are at a percent volume and the percent volumes of the aqueous component, the oil component and the surfactant component sum to 100%; the percent volume of the aqueous component is about 55% to about 65%; the percent volume of the oil component is about 25% to about 35%; and the percent volume of the surfactant component is about 5% to about 15%. In certain instances, the percent volume of the aqueous component is about 60%, the percent volume of the oil component is about 30%, and the percent volume of the surfactant component is about 10%.

In certain instances, the pharmaceutical composition includes: about 8 mg to about 12 mg per mL volume, or about 15 mg to about 25 mg per mL volume, of a compound having the structure of Formula A or Formula B; about 50% to about 70% water by volume, about 20% to about 40% MCT by volume, and about 1% to about 20% PS80 by volume, where the percentage by volume of the water, the percentage by volume of the MCT and the percentage by volume of the PS80 sum to 100%. In certain implementations, the pharmaceutical composition includes: about 8 mg to about 12 mg per mL volume, or about 15 mg to about 25 mg per mL volume, of a compound having the structure of Formula A or Formula B, about 55% to about 65% water by volume, about 25% to about 35% MCT by volume, and about 5% to about 15% PS80 by volume, where the percentage by volume of the water, the percentage by volume of the MCT and the percentage by volume of the PS80 sum to 100%. In certain implementations, the pharmaceutical composition for injection includes: about 1 mM to about 30 mM, or about 5 mM to about 25 mM, or about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula A or Formula B; about 110 mM to about 1170 mM MCT, or about 200 mM to about 1000 mM MCT, or about 300 mM to about 1000 mM MCT, or about 400 mM to about 1000 mM MCT, or about 500 mM to about 900 mM MCT, or about 600 mM to about 800 mM MCT, or about 650 mM to about 750 mM MCT, or about 690 mM to about 700 mM MCT, or about 695 mM to about 700 mM MCT (e.g., about 696 mM MCT, about 697 mM MCT, about 698 mM MCT, about 699 mM MCT); about 40 mM to about 125 mM PS80, or about 60 mM to about 100 mM PS80, or about 70 mM to about 90 mM PS80, or about 75 mM to about 85 mM PS80, or about 79 mM to about 83 mM PS80 (e.g., about 80 mM PS80, about 81 mM PS80, about 82 mM PS80); and sometimes about 55% to about 65% water by volume.

In certain instances, the pharmaceutical composition includes: about 10 mg per mL volume or about 20 mg per mL volume of a compound having a structure according to Formula B, as described herein, about 60% water by volume, about 30% MCT by volume, and about 10% PS80 by volume. In certain implementations, the pharmaceutical composition includes: about 6.9 mM to about 7.3 mM (e.g., about 7.0 mM, about 7.1 mM, about 7.2 mM) or about 13 mM to about 15 mM (e.g., about 14.1 mM, 14.2 mM or about 14.3 mM) of a compound having the structure of Formula B; about 695 mM to about 700 mM MCT (e.g., about 696 mM MCT, about 697 mM MCT, about 698 mM MCT, about 699 mM MCT); about 79 mM to about 83 mM PS80 (e.g., about 80 mM PS80, about 81 mM PS80, about 82 mM PS80); and sometimes about 60% water by volume.

The oil-in-water emulsion pharmaceutical composition for injection typically includes particles. In certain implementations, the particles have one or more of the following characteristics: (i) a mean particle diameter of 200 nm or less, or 150 nm or less, or 100 nm or less, or 75 nm or less, or 50 nm or less. In certain instances, (ii) a standard deviation of the mean particle diameter of about 30 nm or less, or about 25 nm or less, or about 20 nm or less, or about 15 nm or less, or about 10 nm or less, or about 5 nm or less, or about 1 nm or less, or about 0.5 nm or less or about 0.01 nm or less; (iii) a dispersity of about 0.005 to about 0.50, or about 0.01 to about 0.40, or about 0.01 to about 0.30, or about 0.01 to about 0.25, or about 0.10 to about 0.25; or (iv) a standard deviation of the dispersity of about 0.25 or less, about 0.20 or less, about 0.15 or less, about 0.10 or less, about 0.05 or less, about 0.01 or less, about 0.005 or less or about 0.001 or less. In certain implementations, the particles have one or more of the following characteristics: (i) a mean particle diameter of about 100 nm to about 200 nm, or about 130 nm to about 170 nm, or about 140 nm to about 160 nm, or about 150 nm (e.g., about 148.6 nm); (ii) a standard deviation of the mean particle diameter of about 0.5 nm to about 5 nm, or about 0.5 nm to about 4 nm, or about 1 nm to about 3 nm or about 2 nm (e.g., about 1.96); (iii) a dispersity of about 0.05 to about 0.30, or about 0.10 to about 0.20, or about 0.12; (iv) a standard deviation of the dispersity of about 0.005 to about 0.10, or about 0.01 to about 0.03, or about 0.02.

### C. Non-Limiting Example of Oil-In-Water Emulsion Pharmaceutical Composition for Injection

In certain implementations, a pharmaceutical composition includes: an active ingredient including a compound having a structure according to Formula A, a compound having a structure according to Formula B; an aqueous component including water; a surfactant component including a polysorbate (PS); a solvent component including a polyethylene glycol (PEG); and an oil component including Medium Chain Triglycerides (MCT). The PS sometimes includes PS80 or sometimes consists of or consists essentially of PS80. The MCT sometimes includes one or more of C6, C8, C10 and C12 fatty acids, and sometimes one or more or all of the C6, C8, C10 and C12 fatty acids are saturated fatty acids. The MCT sometimes includes a C8 fatty acid and a C10 fatty acid and optionally a C12 fatty acid. The MCT sometimes is from a plant, such as coconut for example. The PEG sometimes includes PEG400 or is sometimes consists of or consists essentially of PEG400.

In certain implementations, the active ingredient contains, consists of or consists essentially of a compound having a structure according to Formula A or Formula B. In certain instances, the active ingredient is at a final concentration of about 1 milligram (mg) to about 20 mg active ingredient per milliliter (mL) volume, or about 8 mg to about 12 mg active ingredient per mL volume, or about 10 mg active ingredient per mL volume, or about 10 mg to about 30 mg active ingredient per mL volume, or about 15 mg to about 25 mg active ingredient per mL volume, or about 18 mg to about 22 mg per mL volume, or about 20 mg active ingredient per mL volume, of the pharmaceutical composition. In certain instances, a pharmaceutical composition for injection includes about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of an active ingredient consisting of or consisting essentially of a compound having the structure according to Formula A or Formula B.

In certain implementations, the pharmaceutical composition includes: an active ingredient containing, consisting of or consisting essentially of a compound having a structure according to Formula A or Formula B; an aqueous component consisting of or consisting essentially of water; a surfactant component consisting of or consisting essentially of PS80; a solvent component consisting of or consisting essentially of PEG400; and an oil component consisting of or consisting essentially of MCT. In certain instances, the aqueous component is at a percent volume of about 30% to about 50%, the oil component is at a percent volume of about 20% to about 40%, the solvent component is at a percent volume of about 5% to about 25%, and the surfactant component is at a percent volume of about 5% to about 25%. In certain instances, the aqueous component, the solvent component, the oil component and the surfactant component each are at a percent volume and the percent volumes of the aqueous component, the solvent component, the oil component and the surfactant component sum to 100%; the percent volume of the aqueous component is about 35% to about 45%; the percent volume of the oil component is about 25% to about 35%; the percent volume of the solvent component is about 10% to about 20%; and the percent volume of the surfactant component is about 10% to about 20%. In certain instances, the percent volume of the aqueous component is about 40%, the percent volume of the oil component is about 30%, the percent volume of the solvent component is about 15%, and the percent volume of the surfactant component is about 15%.

In certain instances, the pharmaceutical composition for injection includes: about 8 mg to about 12 mg per mL volume, or about 15 mg to about 25 mg per mL volume, of a compound having the structure of Formula A or Formula B; about 30% to about 50% water by volume; about 20% to about 40% MCT by volume; about 5% to about 25% PEG400 by volume; and about 5% to about 25% PS80 by volume, where the percentage by volume of the water, the percentage by volume of the MCT, the percentage by volume of the PEG400 and the percentage by volume of the PS80 sum to 100%. In certain instances, the pharmaceutical composition includes: about 8 mg to about 12 mg per mL volume, or about 15 mg to about 25 mg per mL volume, of a compound having the structure of Formula A or Formula B; about 35% to about 45% water by volume; about 25% to about 35% MCT by volume; about 10% to about 20% PEG400 by volume; and about 10% to about 20% PS80 by volume, where the percentage by volume of the water, the percentage by volume of the MCT and the percentage by volume of the PS80 sum to 100%. In certain implementations, the pharmaceutical composition for injection includes: about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula A or Formula B; about 110 mM to about 1170 mM MCT, or about 200 mM to about 1000 mM MCT, or about 300 mM to about 1000 mM MCT, or about 400 mM to about 1000 mM MCT, or about 500 mM to about 900 mM MCT, or about 600 mM to about 800 mM MCT, or about 650 mM to about 750 mM MCT, or about 690 mM to about 700 mM MCT, or about 695 mM to about 700 mM MCT (e.g., about 696 mM MCT, about 697 mM MCT, about 698 mM MCT, about 699 mM MCT); about 80 mM to about 165 mM PS80, or about 90 mM to about 150 mM PS80, or about 110 mM to about 130 mM PS80, or about 115 mM to about 125 mM PS80, or about 119 mM to about 123 mM PS80 (e.g., about 120 mM PS80, about 121 mM PS80, about 121.5 mM PS80, about 122 mM PS80); about 280 mM to about 570 mM PEG400, or about 330 mM to about 500 mM PEG400, or about 370 mM to about 470 mM PEG400, or about 400 mM to about 450 mM PEG400, or about 422 mM to about 426 mM PEG400 (e.g., about 423 mM PEG400, about 424 PEG400, about 425 PEG400); and sometimes about 35% to about 45% water by volume.

In certain instances, the pharmaceutical composition includes: about 10 mg per mL volume or about 20 mg per mL volume of a compound having the structure of Formula B; about 40% water by volume, about 30% MCT by volume; about 15% PEG400 by volume; and about 15% PS80 by volume. In certain implementations, the pharmaceutical composition for injection includes: about 6.9 mM to about 7.3 mM (e.g., about 7.0 mM, about 7.1 mM, about 7.2 mM) or about 13 mM to about 15 mM (e.g., about 14.1 mM, 14.2 mM or about 14.3 mM) of a compound having the structure of Formula A or Formula B; about 695 mM to about 700 mM MCT (e.g., about 696 mM MCT, about 697 mM MCT, about 698 mM MCT, about 699 mM MCT); about 119 mM to about 123 mM PS80 (e.g., about 120 mM PS80, about 121 mM PS80, about 121.5 mM PS80, about 122 mM PS80); about 422 mM to about 426 mM PEG400 (e.g., about 423 mM PEG400, about 424 PEG400, about 425 PEG400); and sometimes about 40% water by volume.

The oil-in-water emulsion pharmaceutical composition for injection typically includes particles. In certain implementations, the particles have one or more of the following characteristics: (i) a mean particle diameter of 200 nm or less, or 150 nm or less, or 100 nm or less, or 75 nm or less, or 50 nm or less. In certain instances, (ii) a standard deviation of the mean particle diameter of about 30 nm or less, or about 25 nm or less, or about 20 nm or less, or about 15 nm or less, or about 10 nm or less, or about 5 nm or less, or about 1 nm or less, or about 0.5 nm or less or about 0.01 nm or less; (iii) a dispersity of about 0.005 to about 0.50, or about 0.01 to about 0.40, or about 0.01 to about 0.30, or about 0.01 to about 0.25, or about 0.10 to about 0.25; or (iv) a standard deviation of the dispersity of about 0.25 or less, about 0.20 or less, about 0.15 or less, about 0.10 or less, about 0.05 or less, about 0.01 or less, about 0.005 or less or about 0.001 or less. In certain implementations, the particles have one or more of the following characteristics: (i) a mean particle diameter of about 1000 nm to about 1400 nm, or about 1100 nm to about 1300 nm, or about 1200 nm to about 1250 nm, or about 1228 nm to about 1229 nm, or about 1228 nm; (ii) a standard deviation of the mean particle diameter of about 1 nm to about 20 nm, or about 8 nm to about 12 nm, or about 9 nm to about 11 nm, or about 10 nm; (iii) a dispersity of about 0.8 to about 1.2, or about 0.95 to about 1.05, or about 1.0; and (iv) a standard deviation of the dispersity of about 0 to about 0.1, or about 0 to about 0.01, or about 0.

### D. Non-Limiting Example of Oil-In-Water Emulsion Pharmaceutical Composition for Injection

In certain implementations, a pharmaceutical composition includes: an active ingredient including a compound having a structure according to Formula A, a compound having a structure according to Formula B; an aqueous component including water; a surfactant component including a polysorbate (PS); a solvent component including a polyethylene glycol (PEG); and an oil component including castor oil (CO). The PS sometimes includes PS80 or sometimes consists of or consists essentially of PS80. The PEG sometimes includes PEG400 or sometimes consists of or consists essentially of PEG400.

In certain implementations, the active ingredient contains, consists of or consists essentially of a compound having a structure according to Formula A or Formula B. In certain instances, the active ingredient is at a final concentration of about 1 milligram (mg) to about 20 mg active ingredient per milliliter (mL) volume, or about 8 mg to about 12 mg active ingredient per mL volume, or about 10 mg active ingredient per mL volume, or about 15 mg to about 25 mg active ingredient per mL volume, or about 18 mg to about 22 mg active ingredient per mL volume, or about 20 mg active ingredient per mL volume, of the pharmaceutical composition. In certain instances, a pharmaceutical composition for injection includes about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure according to Formula A or Formula B.

The pharmaceutical composition sometimes includes: an active ingredient containing, consisting of or consisting essentially of a compound having the structure of Formula A or Formula B; an aqueous component consisting of or consisting essentially of water; a surfactant component consisting of or consisting essentially of PS80; a solvent component consisting of or consisting essentially of PEG400; and an oil component consisting of or consisting essentially of CO. In certain instances, the aqueous component is at a percent volume of about 30% to about 50%, the oil component is at a percent volume of about 20% to about 40%, the solvent component is at a percent volume of about 5% to about 25%, and the surfactant component is at a percent volume of about 5% to about 25%. In certain instances, the aqueous component, the solvent component, the oil component and the surfactant component each are at a percent volume and the percent volumes of the aqueous component, the solvent component, the oil component and the surfactant component sum to 100%; the percent volume of the aqueous component is about 35% to about 45%; the percent volume of the oil component is about 25% to about 35%; the percent volume of the solvent component is about 10% to about 20%; and the percent volume of the surfactant component is about 10% to about 20%. In certain instances, the percent volume of the aqueous component is about 40%, the percent volume of the oil component is about 30%, the percent volume of the solvent component is about 15%, and the percent volume of the surfactant component is about 15%.

The pharmaceutical composition sometimes includes: about 8 mg to about 12 mg per mL volume, or about 15 mg to about 25 mg per mL volume, of a compound having the structure of Formula A or Formula B; about 30% to about 50% water by volume; about 20% to about 40% CO by volume; about 5% to about 25% PEG400 by volume; and about 5% to about 25% PS80 by volume, where the percentage by volume of the water, the percentage by volume of the CO, the percentage by volume of the PEG400 and the percentage by volume of the PS80 sum to 100%. The pharmaceutical composition sometimes includes: about 8 mg to about 12 mg per mL volume, or about 15 mg to about 25 mg per mL volume, of a compound having the structure of Formula A or Formula B; about 35% to about 45% water by volume; about 25% to about 35% CO by volume; about 10% to about 20% PEG400 by volume; and about 10% to about 20% PS80 by volume, where the percentage by volume of the water, the percentage by volume of the CO and the percentage by volume of the PS80 sum to 100%. In certain implementations, the pharmaceutical composition for injection includes: about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula A or Formula B; about 50 mM to about 520 mM CO, or about 100 mM to about 450 mM CO, or about 150 mM to about 400 mM CO, or about 200 mM to about 400 mM CO, or about 250 mM to about 350 mM CO, or about 280 mM to about 320 mM CO, or about 300 mM to about 310 mM CO, or about 305 mM to about 310 mM CO (e.g., about 306 mM CO, about 307 mM CO, about 308 mM CO, about 309 mM CO); about 80 mM to about 165 mM PS80, or about 90 mM to about 150 mM PS80, or about 110 mM to about 130 mM PS80, or about 115 mM to about 125 mM PS80, or about 119 mM to about 123 mM PS80 (e.g., about 120 mM PS80, about 121 mM PS80, about 121.5 mM PS80, about 122 mM PS80); about 280 mM to about 570 mM PEG400, or about 330 mM to about 500 mM PEG400, or about 370 mM to about 470 mM PEG400, or about 400 mM to about 450 mM PEG400, or about 422 mM to about 426 mM PEG400 (e.g., about 423 mM PEG400, about 424 PEG400, about 425 PEG400); and sometimes about 35% to about 45% water by volume.

The pharmaceutical composition for injection sometimes includes: about 10 mg per mL volume or about 20 mg per mL volume of a compound having the structure of Formula B; about 40% water by volume; about 30% CO by volume; about 15% PEG400 by volume; and about 15% PS80 by volume. In certain implementations, the pharmaceutical composition for injection includes: about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula B; about 305 mM to about 310 mM CO (e.g., about 306 mM CO, about 307 mM CO, about 308 mM CO, about 309 mM CO); about 119 mM to about 123 mM PS80 (e.g., about 120 mM PS80, about 121 mM PS80, about 121.5 mM PS80, about 122 mM PS80); about 422 mM to about 426 mM PEG400 (e.g., about 423 mM PEG400, about 424 PEG400, about 425 PEG400); and sometimes about 40% water by volume.

The oil-in-water emulsion pharmaceutical composition for injection typically includes particles. In certain implementations, the particles have one or more of the following characteristics: (i) a mean particle diameter of 200 nm or less, or 150 nm or less, or 100 nm or less, or 75 nm or less, or 50 nm or less. In certain instances, (ii) a standard deviation of the mean particle diameter of about 30 nm or less, or about 25 nm or less, or about 20 nm or less, or about 15 nm or less, or about 10 nm or less, or about 5 nm or less, or about 1 nm or less, or about 0.5 nm or less or about 0.01 nm or less; (iii) a dispersity of about 0.005 to about 0.50, or about 0.01 to about 0.40, or about 0.01 to about 0.30, or about 0.01 to about 0.25, or about 0.10 to about 0.25; or (iv) a standard deviation of the dispersity of about 0.25 or less, about 0.20 or less, about 0.15 or less, about 0.10 or less, about 0.05 or less, about 0.01 or less, about 0.005 or less or about 0.001 or less. In certain implementations, the particles have one or more of the following characteristics: (i) a mean particle diameter of about 100 nm to about 300 nm, or about 180 nm to about 190 nm, or about 185 nm; (ii) a standard deviation of the mean particle diameter of about 0.5 nm to about 10 nm, or about 2 nm to about 4 nm, or about 3 nm; (iii) a dispersity of about 0.005 to about 0.10, or about 0.01 to about 0.03, or about 0.02; and (iv) a standard deviation of the dispersity of about 0.005 to about 0.10, or about 0.01 to about 0.03, or about 0.02.

### Aqueous and Predominantly Aqueous Pharmaceutical Compositions for Injection

In certain implementations, a pharmaceutical composition for injection includes an active ingredient, an aqueous component, and: a solvent component, or a surfactant component, or a solvent component and a surfactant component.

In certain instances, the active ingredient includes a compound having a structure according to Formula A, a compound having a structure according to Formula B, a rimiducid analog (e.g., a compound having a structure of Formula C, D, E, F or G), or a pharmaceutically acceptable slat, ester or amide thereof. In certain implementations, a pharmaceutical composition for injection includes: an active ingredient including a compound having a structure according to Formula A, or a compound having a structure according to Formula B; an aqueous component including water; a solvent component including a polyethylene glycol (PEG); and a surfactant component including a polysorbate (PS). The PEG sometimes includes PEG400 or sometimes consists of or consists essentially of PEG400. The PS sometimes includes PS80 or sometimes consists of or consists essentially of PS80.

In certain instances, the active ingredient is at a final concentration of about 1 milligram (mg) to about 20 mg active ingredient per milliliter (mL) volume, or about 8 mg to about 12 mg active ingredient per mL volume, or about 10 mg active ingredient per mL volume, or about 15 mg to about 25 mg active ingredient per mL volume, or about 18 mg to about 22 mg active ingredient per mL volume, or about 20 mg active ingredient per mL volume. In certain instances, the active ingredient contains, consists of or consists essentially of a compound having a structure of Formula A or Formula B.

In certain implementations, the pharmaceutical composition for injection includes: an active ingredient containing, consisting of or consisting essentially of a compound having a structure according to Formula A, a compound having a structure according to Formula B; an aqueous component consisting of or consisting essentially of water; a solvent component consisting of or consisting essentially of PEG400; and a surfactant component consisting of or consisting essentially of PS80.

In certain instances, the aqueous component is at a percent volume of about 30% to about 50%, the solvent component is at a percent volume of about 20% to about 40%, and the surfactant component is at a percent volume of about 20% to about 40%. In certain instances, the aqueous component, the solvent component and the surfactant component each are at a percent volume, the percent volumes of the aqueous component, the solvent component and the surfactant component sum to 100%, the percent volume of the aqueous component is about 35% to about 45%, the percent volume of the solvent component is about 25% to about 35%, and the percent volume of the surfactant component is about 25% to about 35%. In certain instances, the percent volume of the aqueous component is about 40%, the percent volume of the solvent component is about 30%, and the percent volume of the surfactant component is about 30%.

In certain instances, the pharmaceutical composition for injection includes about 8 mg to about 12 mg per mL volume, or about 15 mg to about 25 mg per mL volume, of a compound having the structure of Formula A or Formula B; about 30% to about 50% water by volume; about 20% to about 40% PEG400 by volume; and about 20% to about 40% PS80 by volume, where the percentage by volume of the water, the percentage by volume of the PEG400 and the percentage by volume of the PS80 sum to 100%. In certain implementations, the pharmaceutical composition for injection includes about 5 mM to about 9 mM, or about 6 mM to about 8 mM, or about 6.5 mM to about 7.5 mM, or about 6.9 mM to about 7.3 mM (e.g., about 7.0 mM, about 7.1 mM, about 7.2 mM), or about 10 mM to about 20 mM, or about 12 mM to about 16 mM, or about 13 mM to about 15 mM (e.g., about 14.1 mM, 14.2 mM or about 14.3 mM) of a compound having the structure of Formula A or Formula B; about 550 mM to about 1150 mM, or about 600 mM to about 1000 mM, or about 700 mM to about 950 mM, or about 800 mM to about 900 mM, or about 830 mM to about 870 mM (e.g., about 840 mM, 850 mM, 860 mM) PEG400; and about 160 mM to about 330 mM, or about 180 mM to about 300 mM, or about 220 mM to about 280 mM, or about 230 mM to about 250 mM, or about 240 mM to about 245 mM (e.g., about 241 mM, about 242 mM, about 243 mM, about 244 mM) PS80, and sometimes about 30% to about 50% water by volume.

In certain instances, a pharmaceutical composition for injection includes about 10 mg per mL volume or about 20 mg per mL volume of a compound having the structure of Formula B; about 40% water by volume; about 30% PEG400 by volume; and about 30% PS80 by volume. In certain implementations, the pharmaceutical composition for injection includes about 6.9 mM to about 7.3 mM (e.g., about 7.0 mM, about 7.1 mM, about 7.2 mM) or about 13 mM to about 15 mM (e.g., about 14.1 mM, 14.2 mM or about 14.3 mM) of a compound having the structure of Formula B; about 830 mM to about 870 mM (e.g., about 840 mM, 850 mM, 860 mM) PEG400; and about 240 mM to about 245 mM (e.g., about 241 mM, about 242 mM, about 243 mM, about 244 mM) PS80, and sometimes about 40% water by volume.

### Predominantly Polar Solvent Pharmaceutical Compositions for Injection

In certain implementations, a predominantly polar solvent pharmaceutical composition for injection contains an active ingredient (e.g., a compound having a structure according to Formula A, a compound having a structure according to Formula B, or a rimiducid analog), and a polar solvent component. A percent volume amount of a polar solvent component in a predominantly polar solvent pharmaceutical composition for injection can be about 50% or greater, or about 60% or greater, or about 70% or greater, or about 80% or greater, or about 85% or greater, or about 90% or greater, or about 95% or greater, or about 99% or greater.

In certain instances, a polar solvent component includes one or more polar solvents. In certain implementations, a polar solvent component includes one or more of NMP, PEG300, PEG400, DMSO, EEE or ethanol. The EEE sometimes is Transcutol®. In a predominantly polar solvent pharmaceutical composition for injection, the active ingredient often is at least 90% pure and sometimes at least 95% pure, and the one or more of the polar solvents often are at least 90% pure and sometimes at least 95% pure.

In certain implementations, a predominantly polar solvent pharmaceutical composition contains no added water, or contains no aqueous component (e.g., saline). In certain implementations, a predominantly polar solvent pharmaceutical composition contains an aqueous component (e.g., water; saline), sometimes in an amount by volume of about 20% (v/v) or less (e.g., about 15% (v/v) or less, about 10% (v/v) or less or about 5% (v/v) or less, of an aqueous component). In certain implementations, a predominantly polar solvent pharmaceutical composition for injection contains no added oil component. In certain implementations, a predominantly polar solvent pharmaceutical composition for injection consists of or consists essentially of the active ingredient and the polar solvent component. A predominantly polar solvent pharmaceutical composition for injection consisting essentially of the active ingredient and the polar solvent component may contain a minor amount of one or more other components that do not materially affect the basic and novel characteristics of the polar solvent component. A component that does not materially affect the basic and novel characteristics of a polar solvent component typically alters the solubility of the active ingredient in the pharmaceutical composition by 5% or less.

An active ingredient in a predominantly polar solvent pharmaceutical composition for injection often includes, consists of, or consists essentially of a compound having a structure according to Formula A, or a compound having a structure according to Formula B, or a rimiducid analog (e.g., a compound having a structure according to Formula C, D, E, F or G, or a pharmaceutically acceptable salt, ester or amide thereof). In certain instances, a compound having a structure of Formula A or Formula B or a rimiducid analog is in a predominantly polar solvent pharmaceutical composition in a weight to weight (wt/wt) amount of about 5 mg/g to about 200 mg/g (e.g., about 10 mg/g or greater, about 15 mg/g or greater, about 20 mg/g or greater, about 25 mg/g or greater, about 30 mg/g or greater, about 35 mg/g or greater, about 40 mg/g or greater, about 45 mg/g or greater, about 50 mg/g or greater, about 55 mg/g or greater, about 60 mg/g or greater, about 65 mg/g or greater, about 70 mg/g or greater, about 75 mg/g or greater, about 80 mg/g or greater, about 85 mg/g or greater, about 90 mg/g or greater, about 95 mg/g or greater, about 100 mg/g or greater, about 105 mg/g or greater, about 110 mg/g or greater, about 115 mg/g or greater, about 120 mg/g or greater, about 125 mg/g or greater, about 130 mg/g or greater, about 140 mg/g or greater, about 150 mg/g or greater, about 160 mg/g or greater, about 170 mg/g or greater, about 180 mg/g or greater, or about 190 mg/g or greater). In certain implementations, a compound having a structure according to Formula A or Formula B or a rimiducid analog is present in a predominantly polar solvent pharmaceutical composition described herein at a concentration of about 5 millimolar (mM) to about 140 mM (e.g., about 10 mM or greater, about 15 mM or greater, about 20 mM or greater, about 25 mM or greater, about 30 mM or greater, about 35 mM or greater, about 40 mM or greater, about 45 mM or greater, about 50 mM or greater, about 55 mM or greater, about 60 mM or greater, about 65 mM or greater, about 70 mM or greater, about 75 mM or greater, about 80 mM or greater, about 85 mM or greater, about 90 mM or greater, about 95 mM or greater, about 100 mM or greater, about 105 mM or greater, about 110 mM or greater, about 115 mM or greater, about 120 mM or greater, about 125 mM or greater, or about 130 mM or greater). In certain implementations, a total volume of a pharmaceutical composition is about 0.1 mL to about 10 mL, or about 0.5 mL to about 5 mL, or about 1 mL to about 5 mL, or about 1 mL to about 3 mL, or about 2 mL.

In certain implementations, a predominantly polar solvent pharmaceutical composition for injection includes one polar solvent, which sometimes is NMP, PEG300, PEG400, DMSO, EEE or ethanol.

In certain implementations, a predominantly polar solvent pharmaceutical composition for injection contains two polar solvents. The polar solvents in the polar solvent composition each are in a percent volume (v/v) amount in the pharmaceutical composition, and the sum of the percent volume of each of the polar solvents in the pharmaceutical composition can be about 100%. In certain instances, a predominantly polar solvent pharmaceutical composition for injection contains about 1% to about 20% (v/v) PEG300 and about 80% to about 99% (v/v) NMP. In certain instances, a predominantly polar solvent pharmaceutical composition for injection contains about 1% to about 50% (v/v) PEG400 and about 50% to about 99% (v/v) NMP. In certain instances, a predominantly polar solvent pharmaceutical composition for injection contains about 90% to about 99% (v/v) NMP and about 1% to about 10% (v/v) DMSO. In certain instances, a predominantly polar solvent pharmaceutical composition for injection contains about 94% to about 99% (v/v) NMP and about 1% to about 6% (v/v) ethanol.

In certain implementations, a predominantly polar solvent pharmaceutical composition for injection contains three polar solvents. The polar solvents in the polar solvent composition each are in a percent volume (v/v) amount in the pharmaceutical composition, and the sum of the percent volume of each of the polar solvents in the pharmaceutical composition can be about 100%. In certain instances, a predominantly polar solvent pharmaceutical composition for injection contains about 10% to about 80% (v/v) NMP, about 1% to about 15% (v/v) DMSO and about 1% to about 15% (v/v) ethanol. In certain instances, a predominantly polar solvent pharmaceutical composition for injection contains about 10% to about 80% (v/v) NMP, about 1% to about 15% (v/v) ethanol and about 1% to about 20% (v/v) PEG300. In certain instances, a predominantly polar solvent pharmaceutical composition for injection contains about 10% to about 80% (v/v) NMP, about 1% to about 15% (v/v) ethanol and about 1% to about 50% (v/v) PEG400.

In certain implementations, a predominantly polar solvent pharmaceutical composition for injection contains four polar solvents. The polar solvents in the polar solvent composition each are in a percent volume (v/v) amount in the pharmaceutical composition, and the sum of the percent volume of each of the polar solvents in the pharmaceutical composition can be about 100%. In certain instances, a predominantly polar solvent pharmaceutical composition for injection contains about 1% to about 20% (v/v) PEG300, about 10% to about 80% (v/v) NMP, about 1% to about 15% (v/v) DMSO and about 1% to about 15% (v/v) ethanol. In certain instances, a predominantly polar solvent pharmaceutical composition for injection contains about 1% to about 50% (v/v) PEG400, about 10% to about 40% (v/v) NMP, about 1% to about 15% (v/v) DMSO and about 1% to about 15% (v/v) ethanol.

### Dosage Form for Administration by Injection

Pharmaceutical forms suitable for injection (e.g., subcutaneous administration) include pharmaceutical forms in sterile aqueous solutions or dispersions and sterile powders for the extemporaneous preparation of sterile injectable solutions or dispersions. An injectable formulation often is sterile and often is fluid. A fluid sometimes is an emulsion, such as an oil-in-water emulsion for example. An injectable pharmaceutical composition typically is stable under the conditions of manufacture and storage and is preserved against the contaminating action of microorganisms, such as bacteria and fungi. A pharmaceutical composition can be delivered to a subject via any suitable injection device, including without limitation, a syringe, needle or microneedle (e.g., including a syringe device for self-administration).

An injectable formulation sometimes includes a carrier, which can be a solvent, excipient, and/or dispersion medium. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, other components described herein, and suitable mixtures thereof. Fluidity of an injectable formulation can be maintained, for example, by the use of a coating, such as lecithin, by the maintenance of the required particle size in the case of dispersion and by the use of surfactants. Prevention of the action of microorganisms can be affected by various antibacterial and antifungal agents, for example, parabens, chlorobutanol, phenol, sorbic acid, thimerosal, and the like. In certain examples, isotonic agents, for example, sugars or sodium chloride may be included. Prolonged absorption of an injectable compositions can be affected by the use in the compositions of agents delaying absorption, for example, aluminum monostearate and gelatin. A pharmaceutical composition may include a co-polymer such as, for example, a co-polymer selected from the group consisting of poly(vinyl alcohol), poly(vinyl pyrrolidone), and hypromellose acetate succinate.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

In certain implementations, a pharmaceutical composition described herein sometimes is in subcutaneous administration dosage form (i.e., for subcutaneous administration) or sometimes is in intramuscular administration dosage form (i.e., for intramuscular administration). A pharmaceutical composition sometimes is in an auto-injector device, which can include a spring-loaded pre-filled syringe. An auto-injector device also is referred to herein as a pen device, and often is a one-use and disposable device. An auto-injector device often includes a cover that covers the needle of the syringe, and often includes a pre-loaded spring as a power source, where the spring and associated mechanical components form a one-shot linear actuator. When triggered an actuator can drive a sequence that includes (i) accelerating the syringe forward, puncturing the injection site; (ii) actuating the piston of the syringe, injecting the pharmaceutical composition; and (iii) deploying a shield that covers the needle. Auto-injection devices are known and non-limiting examples for different pharmaceutical compositions include SureClick® auto-injector; Rebiject®, Rebiject II® and Rebidose® auto-injectors; Anapen®, EpiPen®, Emerade®, and Auvi-Q® auto-injectors; subcutaneous sumatriptan auto-injector.

### Pharmaceutical Compositions for Topical Administration

In certain implementations, such as for formulations for topical administration, including transdermal administration or transmucosal administration (e.g., intranasal, buccal, sublingual, vaginal or rectal administration) for example, a pharmaceutical composition includes an active ingredient and a solubilizing excipient component, wherein: the active ingredient includes a compound having a structure according to Formula A, a compound having a structure according to Formula B, a rimiducid analog (e.g., a compound having a structure according to Formula C, D, E, F or G, or a pharmaceutically acceptable salt, ester or amide thereof); and the solubilizing excipient compound includes one or more of ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG). The EEE sometimes is Transcutol®.

In certain instances, a solubilizing excipient component includes two components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG). A solubilizing excipient sometimes includes a first component and a second component at a ratio of about 40:60 (v:v) to about 60:40 (v:v), or about 50:50 (v:v). A solubilizing excipient component sometimes includes ethyl acetate and ethanol, and sometimes includes IPM and ethanol.

In certain implementations, a solubilizing excipient component includes three components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG). In certain instances, a solubilizing excipient component includes four components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

In a hypothetical solubilizing excipient that includes three components a, b and c, component a may be at a ratio of about a″ that is between about ratio a′ to about ratio a″, component b may be at a ratio of about b″ that is between about ratio b′ to about ratio b″ and component c may be at a ratio of about c′″ that is between about ratio c′ to about ratio c″, where the ratio of each of component is expressed by volume with respect to the volume of the other components in the solubilizing excipient component. As used herein, such ratios of components a, b and c can be expressed as “a′-b″:b′-b″:c′-c″, v:v:v” and can be expressed as “a′:b′″:c′″, v:v:v.” A solubilizing excipient component sometimes includes ethyl acetate, ethanol, DMI and EEE. In certain instances, ethyl acetate is at a ratio of about 20 to about 30, ethanol is at a ratio of about 20 to about 30, DMI is at a ratio of about 10 to about 20, and EEE is at a ratio of about 30 to about 40, by volume with respect to the volume of the other components in the solubilizing excipient component, which is expressed herein as “20-30:20-30:10-20:30-40, v:v:v:v.” In certain implementations, ethyl acetate is at a ratio of about 25, ethanol is at a ratio of about 25, DMI is at a ratio of about 15, and EEE is at a ratio of about 35, by volume with respect to the volume of the other components in the solubilizing excipient component, which is expressed herein as “25:25:15:35, v:v:v:v.”

In certain instances, a solubilizing excipient component includes IPM, ethanol, EEE and DMI, sometimes at ratios 30-40:10-20:30-40:10-20, v:v:v:v:v, and sometimes at ratios 35:15:35:15, v:v:v:v. Sometimes a solubilizing excipient component includes CCT, ethanol, EEEE and DMI, sometimes at ratios 30-40:10-20:30-40:10-20, v:v:v:v, and sometimes at ratios 35:15:35:15, v:v:v:v.

In certain implementations, a solubilizing excipient component includes IPM, PG, EEE, and DMI, sometimes at ratios 35-45:5-15:30-40:10-20, v:v:v:v, and sometimes at ratios 40:10:35:15, v:v:v:v. Sometimes a solubilizing excipient component includes ethyl acetate, IPM, ethanol, DMI and EEE, sometimes at ratios 10-20:10-20:20-30:10-20:25-35, v:v:v:v, and sometimes at ratios 15:15:25:15:30, v:v:v:v.

In certain implementations, a solubilizing excipient component includes five components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG). Sometimes a solubilizing excipient component includes CCT, ethanol, DMI, EEE and PG, sometimes at ratios 20-30:20-30:10-20:25-35:1-10, v:v:v:v:v, and sometimes at ratios 25:25:15:30:5, v:v:v:v:v.

In certain implementations, a pharmaceutical composition includes isopropyl myristate (20-50 wt %), ethanol (5-25 wt %), EEE (25-50 wt %), dimethyl isosorbide (5-25 wt %) and a compound having a structure according to Formula B (2-15 wt %), which sometimes is in dosage form and sometimes is for topical administration. The term “wt %” as used in the foregoing sentence is a percentage of the weight of a specified component to the total weight of active ingredient(s) and additives in the pharmaceutical composition.

A pharmaceutical composition may be incorporated into any suitable dosage form (e.g., unit dosage form), such as tablets, capsules, gel capsules, liquid syrups, soft gels, suppositories, enemas, in syringes or in microneedles for example. A pharmaceutical composition may be formulated as a suspension in aqueous, non-aqueous, or mixed media. Aqueous suspensions may further contain substances that increase viscosity, including for example, sodium carboxymethylcellulose, sorbitol, and/or dextran. A suspension may also contain one or more stabilizers. An amount of active ingredient required for use in treatment will vary not only with the particular form selected (e.g., the salt selected) but also with route of administration, the nature of the condition being treated and the age and condition of the patient and ultimately will be at the discretion of the attendant physician or clinician.

For topical administration, a pharmaceutical composition may be applied in liquid form. Compounds often are administered as compositions or formulations, in combination with a dermatologically acceptable carrier, which may be a solid or a liquid. Compounds may be formulated with a solid carrier, which include finely divided solids such as talc, clay, microcrystalline cellulose, silica, alumina and the like. Useful liquid carriers include water, alcohols or glycols or water-alcohol/glycol blends, or phospholipids in propylene glycol/ethylene glycol, in which the present compounds can be dissolved or dispersed at effective levels, optionally with the aid of non-toxic surfactants. A composition sometimes includes a diluent and sometimes a carrier (e.g., assimilable, editable), buffer, preservative and the like. Additives such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

### Dosage of Active Ingredient in a Pharmaceutical Composition

A dosage of an active ingredient often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD₅₀ (the dose lethal to 50% of the population) and the ED₅₀ (the dose therapeutically effective in 50% of the population) of an active ingredient. The dose ratio between a toxic and therapeutic effect is the therapeutic index and it can be expressed as the ratio ED₅₀/LD₅₀. A dosage of an active ingredient often lies within a range of circulating concentrations for which the ED₅₀ is associated with little or no toxicity. The dosage may vary within this range depending upon the dosage form employed and the route of administration utilized. A therapeutically effective dose of an active ingredient can be estimated initially from cell culture assays. A dose sometimes is formulated to achieve a circulating plasma concentration range covering the IC₅₀ (i.e., the concentration of an active ingredient that achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” levels of an active ingredient in the serum of the test subject. Such assays may utilize antibody mimics and/or “biosensors” generated by molecular imprinting techniques. The active ingredient is used as a template, or “imprinting molecule”, to spatially organize polymerizable monomers prior to their polymerization with catalytic reagents. Subsequent removal of the imprinted molecule leaves a polymer matrix that contains a repeated “negative image” of the active ingredient and is able to selectively rebind the molecule under biological assay conditions (see, e.g., Ansell, et al., Current Opinion in Biotechnology (1996) 7:89-94 and in Shea, Trends in Polymer Science (1994) 2:166-173).

Such “imprinted” affinity matrixes are amenable to ligand-binding assays, whereby the immobilized monoclonal antibody component is replaced by an appropriately imprinted matrix (see, e.g., Vlatakis, et al., Nature (1993) 361:645-647). Through the use of isotope-labeling, “free” concentration of an active ingredient can be readily monitored and used in calculations of IC₅₀. Such “imprinted” affinity matrixes can also be designed to include fluorescent groups whose photon-emitting properties measurably change upon local and selective binding of an active ingredient. These changes can be readily assayed in real time using appropriate fiberoptic devices, in turn allowing the dose in a test subject to be quickly optimized based on its individual IC50. An example of such a “biosensor” is discussed in Kriz, et al, Analytical Chemistry (1995) 67:2142-2144.

Non-limiting examples of doses include milligram or microgram amounts of an active ingredient per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific active ingredient employed, the age, body weight, general health, gender, and diet of the subject, the time of administration, the route of administration, the rate of excretion, any drug combination, and the degree of expression or activity to be modulated.

### Use of Pharmaceutical Compositions

A pharmaceutical composition described herein can be administered to cells to which a therapeutic agent has been administered. In some embodiments, a therapeutic agent includes a polynucleotide encoding a polypeptide that interacts with a multimerizer active ingredient (also referred to as a “multimerizing agent”) in a pharmaceutical composition described herein. In certain implementations, a polypeptide encoded by a polynucleotide of a therapeutic agent can facilitate cell elimination. In certain instances, a polypeptide encoded by a polynucleotide of a therapeutic agent can facilitate cell activation (e.g., immunogenic cell activation). A therapeutic agent sometimes includes a polynucleotide that encodes a chimeric polypeptide, which also is referred to herein as a fusion protein. A chimeric polypeptide sometimes is a non-naturally occurring polypeptide, or exogenous polypeptide, and sometimes is a polypeptide containing two or more different polypeptides or portions thereof that are not joined in nature. A chimeric polypeptide often includes a polypeptide portion that interacts with (e.g., binds to) a multimerizer active ingredient in a pharmaceutical composition described herein. A chimeric polypeptide often can multimerize upon interaction with a multimerizer in a pharmaceutical composition described herein (referred to as “induced multimerization”). A chimeric polypeptide may be inactive or exhibit low baseline activity, and activity of the chimeric polypeptide can be induced and/or significantly increased by induced multimerization of two or more molecules of the chimeric polypeptide (e.g., in a cell).

Multimerization of a chimeric polypeptide in a cell can be facilitated by contacting the cell with a multimerizing active ingredient in a pharmaceutical composition described herein. A cell expressing a chimeric polypeptide may be contacted by a multimerizing agent by administering a multimerizing agent to a cell (e.g., administering a multimerizing agent to a population of cells containing one or more cells expressing a chimeric polypeptide) or to a subject containing the cell. A multimerizing agent administered to induce a chimeric polypeptide activity can be in a pharmaceutical composition described herein. In certain implementations, the multimerizer (i.e., multimerizing agent) is a compound having a structure of Formula A (rimiducid family compound), a compound having a structure of Formula B (rimiducid), a rimiducid analog (e.g., a compound having a structure according to Formula C, D, E, F or G, or a pharmaceutically acceptable salt, ester or amide thereof).

Provided in certain implementations is a method that includes administering a pharmaceutical composition described herein to cells containing a polypeptide (e.g., a chimeric polypeptide, a fusion protein) encoded by a polynucleotide of a therapeutic agent, where the polypeptide is capable of interacting with an active ingredient in the pharmaceutical composition. In certain instances, provided is a method that includes administering a pharmaceutical composition described herein to a subject in need thereof, where cells of the subject (e.g., a subset of cells in the subject) contain a polypeptide (e.g., a chimeric polypeptide, a fusion protein) encoded by a polynucleotide of a therapeutic agent and the polypeptide is capable of interacting with a multimerizing active ingredient in the pharmaceutical composition. A pharmaceutical composition may be administered in vitro, ex vivo or in vivo, and a may include as a multimerizing active ingredient a compound having a structure of Formula A (rimiducid family molecule), a compound having a structure of Formula B (rimiducid), a rimiducid analog (e.g., a compound having a structure according to Formula C, D, E, F or G, or a pharmaceutically acceptable salt, ester or amide thereof).

In some embodiments, a therapeutic agent includes a polynucleotide encoding a polypeptide capable of inducing cell death, such as a pro-apoptotic (or apoptosis-facilitating) polypeptide, for example. In certain implementations, a therapeutic agent includes a polynucleotide encoding a chimeric polypeptide that includes a pro-apoptotic (or apoptosis-facilitating) polypeptide portion. The term pro-apoptotic polypeptide generally refers to a polypeptide that can directly or indirectly induce, promote, and/or cause apoptosis (i.e., programmed cell death). Pro-apoptotic polypeptides generally participate in a cascade that is triggered in response to pro-apoptotic signals, which culminates in cleavage of certain proteins/polypeptides, resulting in disassembly of the cell. Non-limiting examples of pro-apoptotic polypeptides include caspases (e.g., caspase-1, caspase-2, caspase-3, caspase-6, caspase-7, caspase-8, caspase-9, caspase-10, caspase-11, caspase-12), apoptosis-inducing factor (AIF), amyloid-beta-peptide, the pro-apoptotic bcl-2 subfamily of proteins (e.g., BAX, BID, BAK, BAD), granzymes (e.g., granzyme A, B), Caspase-binding adapter molecules (e.g., apoptotic protease activating factor 1 (APAF1), Fas, Fas-associated death domain (FADD)-containing protein, and death domain-containing protein (CRADD)), p53, death domain-containing members of the tumor necrosis factor receptor (TNFR) family (e.g., Fas and TNFR1) and certain heat shock proteins. Non-limiting examples of polypeptides capable of facilitating cell elimination are described in Savrou et al., Molecular Therapy 26(5), 1266-1276 (2018); Duong et al., Molecular Therapy: Oncolytics 12, 124-137 (2019); and U.S. Patent Application Publication No. US20160166613A1, each of which is incorporated by reference herein.

In some embodiments, a therapeutic agent includes a senolytic agent. The term senolytic agent generally refers to an agent that can selectively induce, promote, and/or cause death (e.g., apoptosis (i.e., programmed cell death)) of senescent cells. In some embodiments, a therapeutic agent includes a polynucleotide encoding a senolytic polypeptide or a functional variant or fragment thereof. In certain implementations, a therapeutic agent includes a polynucleotide encoding a chimeric polypeptide that includes a senolytic polypeptide portion. In some embodiments, a therapeutic agent includes a senescent cell-specific promoter. For example, a therapeutic agent may include a nucleic acid construct containing (1) a polynucleotide encoding pro-apoptotic polypeptide and (2) a senescent cell-specific promoter. Senescent cell-specific promoters may include one or more regulatory regions (e.g., a promoter region, or variant thereof, or fragment thereof) from a gene that is upregulated in senescent cells compared to non-senescent cells. Genes that are upregulated in senescent cells include cyclin-dependent kinase (CDK) inhibitors (e.g., p16^{INK4a}) cyclin-dependent kinase inhibitor 1 (p21^{Cip1}), senescence-associated β-galactosidase (SA-β-gal), transcription factor, E2F7, transcription factor regulator, BTG2, and others. A senolytic agent may be useful for treating one or more skin conditions, skin diseases, skin lesions, and/or skin disorders. For example, a senolytic agent may be used to treat actinic keratosis, Bowen's disease, and the like.

In some embodiments, a therapeutic agent includes a polynucleotide encoding a caspase. In some embodiments, a therapeutic agent includes a polynucleotide encoding caspase-9. Caspase-9 is an enzyme encoded by the CASP9 gene (in humans). Caspase-9 belongs to a family of caspases, cysteine-aspartic proteases involved in apoptosis and cytokine signaling. Apoptotic signals promote the release of cytochrome c from mitochondria and activation of apaf-1 (apoptosome), which then cleaves the pro-enzyme of caspase-9 into the active dimer form. Caspase-9 has three domains: N-terminal pro-domain, large subunit, and a small subunit. The N-terminal pro-domain contains the caspase activation domain (CARD) motif and is linked to a catalytic domain by a linker loop. The large subunit and small subunit both include the catalytic domain. In some embodiments, a therapeutic agent includes a polynucleotide encoding a caspase-9 polypeptide fragment lacking a CARD domain.

In some embodiments, a therapeutic agent includes a polynucleotide encoding an inducible caspase-9, which is referred to herein as “iCasp9” or “iC9”. In certain implementations, a therapeutic agent includes a polynucleotide encoding a chimeric polypeptide containing an inducible caspase-9 polypeptide portion. In some embodiments, a therapeutic agent includes a polynucleotide encoding an inducible caspase-9 (iCasp9) polypeptide fragment lacking a CARD domain. An inducible caspase 9 may be generated by modifying caspase-9 and fusing it with a polypeptide that binds to a multimerizing agent in a chimeric polypeptide (e.g., fusion protein). Such a modification sometimes is performed by linking a first polynucleotide encoding a caspase-9 to a second polynucleotide encoding a polypeptide that binds to a multimerizing agent. The first polynucleotide may be linked directly to the second polynucleotide or may be linked via a linker, for example. A non-limiting example of a polypeptide that binds to a multimerizing agent is FK506-binding protein or variant thereof (e.g., FKBP12, FKBP12-V36, FRB or variant of the foregoing). A multimerizing agent can bind to the protein fused to the modified caspase-9, dimerizing and activating caspase-9, which triggers cell death. In some embodiments, an inducible caspase-9 is induced by a multimerizing agent (e.g., a dimerizer).

In some embodiments, a therapeutic agent includes a polynucleotide encoding a homodimerizer-based inducible caspase-9 (iCasp9). Non-limiting examples of inducible caspase-9 systems are described in Fan et al., Hum. Gene Ther. 10(14):2273-85 (1999); and Straathof et al., Blood 105(11):4247-54 (2005), each of which is incorporated by reference herein. In some embodiments, a therapeutic agent includes a polynucleotide encoding an inducible caspase-9 (iCasp9) construct (e.g., chimeric polypeptide, fusion protein). Non-limiting examples of inducible caspase-9 (iCasp9) construct components are provided in the Table A below.

In some embodiments, a therapeutic agent includes a polynucleotide encoding an inducible caspase-9 (iCasp9) construct comprising a first polypeptide and a second polypeptide (i.e., chimeric polypeptide, fusion protein). In some embodiments, the first polypeptide is a FKBP12-V36 polypeptide. In some embodiments, the first polypeptide is a human FKBP12-V36 polypeptide. In some embodiments, the first polypeptide includes a polypeptide that is at least 90% identical to SEQ ID NO:1. In some embodiments, the first polypeptide includes a polypeptide that is at least 95% identical to SEQ ID NO:1. In some embodiments, the first polypeptide includes a polypeptide that is at least 99% identical to SEQ ID NO:1. In some embodiments, the first polypeptide includes the polypeptide of SEQ ID NO:1. In some embodiments, the second polypeptide is a dCasp9 (5′ truncated Casp9) polypeptide. In some embodiments, the second polypeptide is a dCasp9 (5′ truncated human Casp9) polypeptide. In some embodiments, the second polypeptide includes a polypeptide that is at least 90% identical to SEQ ID NO:3. In some embodiments, the second polypeptide includes a polypeptide that is at least 95% identical to SEQ ID NO:3. In some embodiments, the second polypeptide includes a polypeptide that is at least 99% identical to SEQ ID NO:3. In some embodiments, the second polypeptide includes the polypeptide of SEQ ID NO:3. In some embodiments, a linker is located between the first polypeptide and the second polypeptide. In some embodiments, the linker is a GS linker. In some embodiments, the linker includes about 1, 2, 3, 4, 5, 6, 7, 8, 9, or 10 amino acids. In some embodiments, the linker includes about 6 amino acids. In some embodiments, the linker includes the peptide of SEQ ID NO:2.

A chimeric polypeptide (e.g., fusion protein) encoded by a therapeutic agent nucleic acid sometimes includes a FKBP12-V36 portion that lacks an N-terminal methionine when the FKBP12-V36 portion is not located at the N-terminus of the chimeric polypeptide (SEQ ID NO:4).

In certain implementations, a therapeutic agent encodes or includes a binding domain that binds to rapamycin or a rapamycin analog, such as a FKBP-rapamycin-binding (FRB) domain, for example. In certain implementations, the first polypeptide is located at the N-terminus of the chimeric polypeptide (e.g., fusion protein), the first polypeptide is located closer to the N-terminus of the chimeric polypeptide (e.g., fusion protein) than the second polypeptide, the second polypeptide is located closer to the N-terminus of the chimeric polypeptide (e.g., fusion protein) than the first polypeptide, or the second polypeptide is located at the C-terminus of the chimeric polypeptide.

In certain implementations, a therapeutic agent includes a polynucleotide encoding a chimeric polypeptide (e.g., fusion protein) that includes (i) a polypeptide portion capable of interacting with (e.g., binding to) a multimerizing agent, and (ii) one or more polypeptide portions capable of activating a cell, such as an immune system cell. Non-limiting examples of immune system cells include T-cells (e.g., alpha-beta T-cells, gamma-delta T-cells), NK cells and the like. A chimeric polypeptide (e.g., fusion protein) can include a suitable type of polypeptide portion capable of activating a cell, such as an immune cell, non-limiting examples of which include CD3-zeta, CD19, 41BB, OX40, MyD88, CD40, a portion thereof and combinations of the foregoing. In certain implementations, a chimeric polypeptide (e.g., fusion protein) includes MyD88 or a truncated form of MyD88. A MyD88 sometimes is a polypeptide product of the myeloid differentiation primary response gene 88 (NCBI Gene ID 4615). A truncated MyD88 polypeptide is shorter than the full-length MyD88 polypeptide and sometimes lacks a domain or portion of a domain, such as the TIR domain, for example. A non-limiting example of a truncated MyD88 polypeptide has a structure according to SEQ ID NO:5. In certain implementations, a chimeric polypeptide (e.g., fusion protein) includes CD40 (e.g., human CD40) or a truncated form of CD40. A truncated CD40 polypeptide is shorter than the full-length CD40 polypeptide and sometimes lacks a domain, such as the extracellular domain and/or transmembrane domain, or portion of such a domain, for example. A non-limiting example of a truncated CD40 polypeptide referred to as “cytoplasmic CD40” has a structure according to SEQ ID NO:6.

A chimeric polypeptide (e.g., fusion protein) encoded by a therapeutic agent nucleic acid sometimes includes a first polypeptide, a second polypeptide and a third polypeptide. In some embodiments, the first polypeptide is a truncated MyD88 polypeptide or MyD88 polypeptide. In some embodiments, the first polypeptide is a truncated human MyD88 polypeptide. In some embodiments, the first polypeptide includes a polypeptide that is at least 90% identical to SEQ ID NO:5. In some embodiments, the first polypeptide includes a polypeptide that is at least 95% identical to SEQ ID NO:5. In some embodiments, the first polypeptide includes a polypeptide that is at least 99% identical to SEQ ID NO:5. In some embodiments, the first polypeptide includes the polypeptide of SEQ ID NO:5. In some embodiments, the second polypeptide is a CD40 polypeptide or cytoplasmic CD40 polypeptide. In some embodiments, the second polypeptide is a human cytoplasmic CD40 polypeptide. In some embodiments, the second polypeptide includes a polypeptide that is at least 90% identical to SEQ ID NO:6. In some embodiments, the second polypeptide includes a polypeptide that is at least 95% identical to SEQ ID NO:6. In some embodiments, the second polypeptide includes a polypeptide that is at least 99% identical to SEQ ID NO:6. In some embodiments, the second polypeptide includes the polypeptide of SEQ ID NO:6. In some embodiments, the third polypeptide is a FKBP12-V36 polypeptide. In some embodiments, the third polypeptide is a human FKBP12-V36 polypeptide. In some embodiments, the third polypeptide includes a polypeptide that is at least 90% identical to SEQ ID NO:4. In some embodiments, the third polypeptide includes a polypeptide that is at least 95% identical to SEQ ID NO:4. In some embodiments, the third polypeptide includes a polypeptide that is at least 99% identical to SEQ ID NO:4. In some embodiments, the third polypeptide includes the polypeptide of SEQ ID NO:4. In certain implementations, a therapeutic agent encodes or includes a binding domain that binds to rapamycin or a rapamycin analog, such as a FKBP-rapamycin-binding (FRB) domain, for example. In certain embodiments, the cytoplasmic polypeptide (e.g., fusion protein) includes the following polypeptides in the following order: N-terminus—first polypeptide—second polypeptide—third polypeptide—C-terminus, where there optionally is a linker between the first polypeptide and the second polypeptide and there optionally is a linker between the second polypeptide and the third polypeptide. Any suitable linker can be incorporated, such as a linker containing about 10 or fewer contiguous amino acids (e.g., a linker that includes about 1, 2, 3, 4, 5, 6, 7, 8, 9, or 10 amino acids), non-limiting examples which include a GS linker (e.g., SEQ ID NO:2), a linker containing or consisting of the amino acids LN, RT, VG (designated by the one-letter amino acid code), and the like.

A chimeric polypeptide (e.g., fusion protein) encoded by a therapeutic agent nucleic acid sometimes includes a polypeptide portion that associates with a portion of a cell membrane, which can facilitate membrane localization and multimerization of multiple molecules of the chimeric polypeptide. A non-limiting example of a membrane association polypeptide portion is a myristoyl-association polypeptide portion, which can have structure according to SEQ ID NO: 7. In certain implementations, a chimeric polypeptide (e.g., fusion protein) includes a myristoyl-association polypeptide at least 90% identical to SEQ ID NO:7, or a myristoyl-association polypeptide at least 95% identical to SEQ ID NO:7, or a myristoyl-association polypeptide at least 99% identical to SEQ ID NO:7, or the myristoyl-association polypeptide of SEQ ID NO:7. A membrane-association polypeptide portion of a chimeric polypeptide (e.g., fusion protein) sometimes is located at the N-terminus of the chimeric polypeptide, and sometimes is spaced from an adjacent domain in the chimeric polypeptide by a linker containing 10 or fewer contiguous amino acids (e.g., a GS linker (e.g., SEQ ID NO:2), or a linker containing or consisting of LN, RT, VG, and the like).

TABLE A

| Polypeptide | |
|---|---|
| element | Polypeptide sequence |
| human | MGVQVETISPGDGRTFPKRGQTCVV |
| FKBP12-V36 | HYTGMLEDGKKVDSSRDRNKPFKFM |
| | LGKQEVIRGWEEGVAQMSVGQRAKL |
| | TISPDYAYGATGHPGIIPPHATLVF |
| | DVELLKLE |
| | (SEQ ID NO: 1) |
| GS linker | SGGGSG (SEQ ID NO: 2) |
| dCasp9 (5′ | GFGDVGALESLRGNADLAYILSMEP |
| truncated | CGHCLIINNVNFCRESGLRTRTGSN |
| human | IDCEKLRRRFSSLHFMVEVKGDLTA |
| Casp9) | KKMVLALLELARQDHGALDCCVVVI |
| | LSHGCQASHLQFPGAVYGTDGCPVS |
| | VEKIVNIFNGTSCPSLGGKPKLFFI |
| | QACGGEQKDHGFEVASTSPEDESPG |
| | SNPEPDATPFQEGLRTFDQLDAISS |
| | LPTPSDIFVSYSTFPGFVSWRDPKS |
| | GSWYVETLDDIFEQWAHSEDLQSLL |
| | LRVANAVSVKGIYKQMPGCFNFLRK |
| | KLFFKTSASRA (SEQ ID NO: 3) |
| human | GVQVETISPGDGRTFPKRGQTCVVH |
| FKBP12-V36 | YTGMLEDGKKVDSSRDRNKPFKFML |
| lacking | GKQEVIRGWEEGVAQMSVGQRAKLT |
| N-terminal | ISPDYAYGATGHPGIIPPHATLVFD |
| methionine | VELLKLE(SEQ ID NO: 4) |
| truncated | MAAGGPGAGSAAPVSSTSSLPLAAL |
| MyD88 | NMRVRRRLSLFLNVRTQVAADWTAL |
| | AEEMDFEYLEIRQLETQADPTGRLL |
| | DAWQGRPGASVGRLLDLLTKLGRDD |
| | VLLELGPSIEEDCQKYILKQQQEEA |
| | EKPLQVAAVDSSVPRTAELAGITTL |
| | DDPLGHMPERFDAFICYCPSDI |
| | (SEQ ID NO: 5) |
| cytoplasmic | KKVAKKPTNKAPHPKQEPQEINFPD |
| CD40 | DLPGSNTAAPVQETLHGCQPVTQED |
| | GKESRISVQERQ |
| | (SEQ ID NO: 6) |
| myristoyl- | MGSSKSKPKDPSQR |
| association | (SEQ ID NO: 7) |
| polypeptide | |

In certain implementations, a therapeutic agent polynucleotide encoding a chimeric polypeptide (e.g., iC9) is incorporated in cells using methodology known in the art (e.g., transduction, transfection and the like), and the chimeric polypeptide is encoded in cells by the incorporated polynucleotide. In certain instances, a therapeutic polynucleotide encoding a chimeric polypeptide is incorporated into cell nucleic acid using a known genome editing process. In some embodiments, a therapeutic agent includes a polynucleotide encoding a genome-editing polypeptide, genome-editing enzyme, or genome-editing system. In some embodiments, a therapeutic agent includes a polynucleotide encoding a gene-editing polypeptide, gene-editing enzyme, or gene-editing system. In some embodiments, a gene-editing system includes one or more gene-editing polypeptides, gene-editing enzymes, and/or a gene-editing fusion protein. A gene-editing polypeptide, gene-editing enzyme, or gene-editing system generally has one or more capabilities chosen from DNA binding, DNA cleaving, DNA modification, DNA deletion, DNA insertion, and DNA replacement. Often, gene-editing systems target the binding, cleaving, modification, deletion, insertion, and/or replacement to site-specific locations (e.g., specific location(s) in the genome; specific gene(s)). Non-limiting examples of gene-editing polypeptides and/or gene-editing enzymes include CRISPR (clustered regularly interspaced short palindromic repeats) associated proteins (Cas) (e.g., Cas3, Cas9, Cas10), CRISPR-associated endonuclease in Prevotella and Francisella 1 (Cpf1), transcription activator-like effector (TALE) proteins, transcription activator-like effector nucleases (TALENs), zinc finger proteins, endonucleases, integrases, recombinases, transposases, restriction enzymes, hybrid proteins such as a nuclease-inactivated Cas9 polypeptide fused to a Clo51 endonuclease (e.g., Cas-CLOVER), and the like. In some embodiments, a gene-editing system further includes a polynucleotide encoding a guide RNA (gRNA). Guide RNA can help one or more gene-editing enzymes bind specific sequences of DNA in a genome. Guide RNA may be of any suitable length (e.g., about 10 to 50 nucleotides in length, about 15 to 30 nucleotides in length, about 18 to 22 nucleotides in length, or about 20 nucleotides in length). A gene-editing system herein may be useful for treating one or more genetic conditions, genetic diseases, and/or genetic disorders. For example, a gene-editing system herein may be used for treating hair loss/baldness (e.g., by knocking out one or more genes encoding an androgen receptor in cells associated with hair growth (e.g., basal epidermal cells, hair follicle cells).

A pharmaceutical composition sometimes is administered to multimerize a chimeric polypeptide (e.g., fusion protein) that includes a polypeptide that binds to a multimerizing active ingredient (e.g., a compound having a structure of Formula A (rimiducid family molecule), a compound having a structure of Formula B (rimiducid), a rimiducid analog (e.g., a compound having a structure according to Formula C, D, E, F or G, or a pharmaceutically acceptable salt, ester or amide thereof)). A non-limiting example of a polypeptide that can bind to rimiducid (i.e., a compound having a structure according to Formula B) is FKBP12-V36. A FKBP12-V36 polypeptide sometimes includes the polypeptide of SEQ ID NO:1 or SEQ ID NO:4. A chimeric polypeptide (e.g., fusion protein) sometimes includes a polypeptide capable of inducing cell death (e.g., apoptosis), such as a caspase-9 polypeptide, for example. A caspase-9 polypeptide incorporated in chimeric polypeptide (e.g., a fusion protein) sometimes is a truncated caspase-9 polypeptide, such as a polypeptide of SEQ ID NO:3, for example. A chimeric polypeptide (e.g., fusion protein) sometimes includes the polypeptides of SEQ ID NO:1, SEQ ID NO:2 and SEQ ID NO:3, which can be presented in the foregoing order in a chimeric polypeptide (e.g., fusion protein) in the N-terminal to C-terminal direction. A chimeric polypeptide (e.g., fusion protein) often is expressed in cells, where the cells sometimes occur in vitro or ex vivo, or are in vivo.

A pharmaceutical composition sometimes is administered as part of a chimeric antigen receptor (CAR) molecule treatment. In a CAR molecule treatment, a subject often is administered immunologic cells containing a nucleic acid encoding a CAR molecule and/or a CAR molecule encoded by the nucleic acid. Where the immunologic cells administered are T-cells, the treatment typically is referred to as a CAR-T treatment.

A pharmaceutical composition sometimes is administered for elimination of cells containing a fusion protein, wherein the fusion protein contains a polypeptide that binds to the compound having the structure of Formula A, or Formula B or a rimiducid analog and often contains a polypeptide capable of inducing cell death (e.g., apoptosis). A pharmaceutical composition sometimes is administered as part of a treatment of a cell proliferative condition. A cell proliferative condition sometimes is a cancer. Non-limiting examples of cancers include an adrenal gland tumor, an AIDS-associated cancer, an alveolar soft part sarcoma, an astrocytic tumor, bladder cancer, bone cancer, a brain cancer, a spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

A pharmaceutical composition sometimes is administered as part of a treatment of a skin condition. Non-limiting examples of skin conditions include a skin lesion, actinic keratosis, skin carcinoma and Bowen's disease.

A pharmaceutical composition sometimes is administered as part of a treatment of a pathogen condition. A pathogen condition sometimes is a viral condition, fungal condition, parasitic condition or bacterial condition. Non-limiting examples of pathogen conditions include those characterized the presence of Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, or Leishmania donovani.

A pharmaceutical composition can be administered to a mammalian patient or subject, such as a human patient or nonhuman animal, in a variety of forms adapted to the chosen route of administration. The terms “patient” or “subject” are interchangeable, and include without limitation a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

Provided hereafter is a non-limiting representative application of pharmaceutical compositions described herein. The following representative application pertains to eradication of modified cells that express a therapeutic protein in a sub-population of subjects receiving a treatment. In a specific representative application, a large portion of the population has been exposed to an infectious virus. There is an identified monoclonal antibody (MAb) that neutralizes the virus, but there is not enough purified MAb available for the entire at-risk population. Instead, there is a delivery system available that carries nanoparticles (NPs) encapsulating transposase mRNA plus integrating transposon plasmids that encodes the antibody. Upon administration of the particles, MAbs are released systemically at effective levels after genetic modification occurs in cells of the subject. Once the risk of infection has abated or a counterindication occurs for systemic and constitutive MAb production, genetically engineered skin cells may need to be eliminated. In addition, a subset of individuals may present an adverse reaction associated with the genetically modified cells or antibodies they produce. For example, certain individuals may have a rare cross-reactivity to a hepatocyte surface marker due to the MAb. To increase safety and terminate MAb expression when obsolete, an inducible safety switch chimeric polypeptide (e.g., fusion protein), containing FKBP12V36 and a truncated caspase-9 (iC9), is co-expressed with the MAb. To activate the pro-apoptotic iC9 switch, the individuals will be administered, or self-administer, a rimiducid pharmaceutical composition described herein, such as a pharmaceutical composition in unit dosage form for example (e.g., administered by a pen-injection device), leading to apoptosis of genetically modified cells and termination of MAb expression.

### Methods for Manufacturing Pharmaceutical Compositions

Any suitable known method can be used to manufacture a pharmaceutical composition described herein. An active ingredient, such as a rimiducid family compound of a structure of Formula A, rimiducid of a structure of Formula B, or a rimiducid analog (e.g., of a structure of Formula B, C, D, E, F or G, or a pharmaceutically acceptable salt, ester or amide thereof), for example, often is in a purified form. One or more or all components (e.g., excipient components), such as an oil component, solvent component (e.g., polar solvent component), aqueous component, surfactant component, for example, mixed with the active ingredient often are in a purified form. A purified form can be referred to as a refined form, and sometimes an active ingredient and one or more or all components mixed with the active ingredient are in a highly purified or highly refined form. An active ingredient and one or more other components utilized to a prepare a pharmaceutical composition formulation each sometimes is at least 90% pure, and sometimes at least 95% pure (e.g., 90% pure or greater, 91% pure or greater, 92% pure or greater, 93% pure or greater, 94% pure or greater, 95% pure or greater, 96% pure or greater, 97% pure or greater, 98% pure or greater, 99% pure or greater, 99.5% pure or greater or 99.9% pure or greater). A percent purity of an active ingredient or other component typically is an amount of the active ingredient or component in a reagent utilized to prepare a pharmaceutical composition, relative to other elements that may be present in the reagent. The amount sometimes is by weight, typically weight of the component to the overall weight of the reagent, and sometimes is by volume, typically volume of the component to the overall volume of the reagent. An active ingredient and one or more other components utilized to prepare a pharmaceutical composition formulation each sometimes is clinical grade, American Chemical Society (ACS) grade, Reagent grade, National Formulary (NF) grade, United States Pharmacopeia (USP) grade and/or GMP grade (i.e., manufactured according to Good Manufacturing Practice (GMP) or Current Good Manufacturing Practice (cGMP)).

Provided herein are methods for manufacturing a pharmaceutical composition suitable for administration by injection (e.g., SQ or IM administration). In certain implementations, provided is a method for manufacturing a pharmaceutical composition for injection containing an active ingredient that includes, consists of or consists essentially of a compound having a structure according to Formula A, a compound having a structure according to Formula B, a rimiducid analog (e.g., a compound having a structure according to Formula C, D, E, F or G, or a pharmaceutically acceptable salt, ester or amide thereof). In certain implementations, the method includes: combining the active ingredient with an aqueous component, a surfactant component and an oil component. In certain instances, the method for manufacture includes (a) combining the active ingredient with the oil component, thereby generating an oil mixture; (b) combining the surfactant component with the aqueous component, thereby generating an aqueous mixture; and (c) after (a) and (b), combining the oil mixture with the aqueous mixture.

In certain implementations, the method for manufacture includes: combining the active ingredient with an aqueous component, a solvent component, a surfactant component and an oil component. In certain instances, the method includes (a) combining the active ingredient with the oil component, thereby generating an oil mixture; (b) combining the surfactant component and the solvent component with the aqueous component, thereby generating an aqueous mixture; and (c) after (a) and (b), combining the oil mixture with the aqueous mixture.

In certain implementations, (i) the oil mixture is, or (ii) the aqueous mixture is, or (iii) the oil mixture and the aqueous mixture are, exposed to conditions in which the temperature is above room temperature. In certain instances, the temperature is about 40 degrees Celsius to about 80 degrees Celsius, or about 50 degrees Celsius to about 70 degrees Celsius, or about 60 degrees Celsius.

For implementations in which an oil component is utilized, a composition prepared often is an oil-in-water emulsion including particles. For example, a composition prepared by mixing an oil mixture with an aqueous mixture often is an oil-in-water emulsion including particles. In certain implementations, an emulsion containing particles is formed, and the method of manufacture does not include exposing the particles to conditions that reduce the size of the particles. In certain implementations, an emulsion containing particles is formed, and the method of manufacture includes exposing the particles to conditions that reduce the size of the particles. In the latter implementations, any suitable conditions that reduce the size of the particles can be utilized, non-limiting examples of which include sonication conditions, micro-fluidization conditions, ball-milling conditions, or the like, or a combination thereof.

In certain manufacturing processing implementations, which can include or not include exposing particles to conditions that reduce the size of particles, the particles in an intermediate composition, or in a final pharmaceutical composition, can have one or more of the following characteristics: (i) a mean particle diameter of 200 nm or less, or 150 nm or less, or 100 nm or less, or 75 nm or less, or 50 nm or less. In certain instances, (ii) a standard deviation of the mean particle diameter of about 30 nm or less, or about 25 nm or less, or about 20 nm or less, or about 15 nm or less, or about 10 nm or less, or about 5 nm or less, or about 1 nm or less, or about 0.5 nm or less or about 0.01 nm or less; (iii) a dispersity of about 0.005 to about 0.50, or about 0.01 to about 0.40, or about 0.01 to about 0.30, or about 0.01 to about 0.25, or about 0.10 to about 0.25; or (iv) a standard deviation of the dispersity of about 0.25 or less, about 0.20 or less, about 0.15 or less, about 0.10 or less, about 0.05 or less, about 0.01 or less, about 0.005 or less or about 0.001 or less.

In certain implementations of a method of manufacture that includes processing an oil component, an oil component can include one or more of medium-chain triglycerides (MCT), castor oil (CO), medium-chain triglycerides of caprylic (C8) and capric (C10) acids (CAP), oleoyl polyoxyl-6 glycerides (OPG), polyethoxylated castor oil (PECO), Triacetin, oleic acid (OA), propylene glycol monocaprylate (PGMC) (Type II), glycerol monooleate (GMO), caprylocaproyl polyoxyl-8 glycerides (CCPG), propylene glycol monolaurate (PGML) Type II, glyceryl monolinoleate (GML) and Pluronic® polymers (PP). In certain instances, an oil component includes MCT, or consists of MCT or consists essentially of MCT. In certain instances, an oil component includes CO, consists of CO or consists essentially of CO. In certain implementations of a method of manufacture that includes processing a solvent component, a solvent component sometimes includes a polyethylene glycol (PEG), or sometimes consists of or consists essentially of a polyethylene glycol (PEG). Sometimes the PEG is PEG400. In certain implementations, an aqueous component includes water or saline, or consists of water or consists essentially of water. In certain implementations, a surfactant component includes a polysorbate (PS). A PS utilized sometimes includes PS 20, 40, 60 and/or 80. A PS utilized sometimes includes PS80, or sometimes consists of PS80 or consists essentially of PS80. Specific combinations of components (e.g., active ingredient, aqueous component, surfactant component, oil component, solvent component) can combined in amounts described herein in specific methods for manufacturing pharmaceutical compositions. Provided in certain implementations are pharmaceutical compositions obtainable by methods of manufacture described herein.

In certain implementations, provided is a method for manufacturing a pharmaceutical composition for injection, the method including: combining an active ingredient with an aqueous component, a solvent component and a surfactant component, where the active ingredient includes, consists of or consists essentially of a compound having a structure according to Formula A, a compound having a structure according to Formula B, a rimiducid analog (e.g., a compound having a structure according to Formula C, D, E, F or G, or a pharmaceutically acceptable salt, ester or amide thereof).

In certain implementations, a method for manufacturing a pharmaceutical composition includes: combining an active ingredient with one or more polar solvents, where the active ingredient contains a compound having the structure of Formula A, a compound having the structure of Formula B, or a rimiducid analog. In certain instances, the method includes (a) combining two or more polar solvents, thereby generating a first mixture; and (b) combining the active ingredient with the polar solvent mixture, thereby generating a second mixture. In certain instances, the first mixture, or the second mixture, or the first mixture and the second mixture are exposed to: conditions in which the temperature is above room temperature, or agitation conditions, or agitation conditions and conditions in which the temperature is above room temperature. In certain instances, the temperature is about 40 degrees Celsius to about 60 degrees Celsius or about 50 degrees Celsius. In certain instances, the first mixture, or the second mixture, or the first mixture and the second mixture, is exposed to conditions in which the temperature is above room temperature for about 1 hour to about 5 hours. In certain instances, the first mixture, or the second mixture, or the first mixture and the second mixture, is exposed to conditions in which the temperature is above room temperature for about 3 hours. In certain instances, the first mixture, or the second mixture, or the first mixture and the second mixture, is exposed to agitation conditions for about 12 hours to about 36 hours, or about 24 hours.

In certain implementations, a method for manufacturing a pharmaceutical composition includes: combining an active ingredient with an oil component and one or more polar solvents, where the active ingredient contains a compound having the structure of Formula A, a compound having the structure of Formula B, or a rimiducid analog. In certain instances, the method includes (a) combining the active ingredient with the polar solvent mixture, thereby generating a first mixture; and (b) combining the first mixture with the oil component, thereby generating a second mixture. In certain instances, the first mixture, or the second mixture, or the first mixture and the second mixture, is exposed to: conditions in which the temperature is above room temperature, or agitation conditions, or agitation conditions and conditions in which the temperature is above room temperature. In certain instances, the temperature is about 40 degrees Celsius to about 60 degrees Celsius, or about 50 degrees Celsius. In certain instances, the first mixture, or the second mixture, or the first mixture and the second mixture, is exposed to conditions in which the temperature is above room temperature for about 1 hour to about 5 hours, or about 3 hours. In certain instances, the first mixture, or the second mixture, or the first mixture and the second mixture, is exposed to agitation conditions for about 12 hours to about 36 hours, or about 24 hours.

Certain implementations of methods for manufacturing pharmaceutical compositions include preparing a pharmaceutical composition in a dosage form. A dosage form sometimes is a subcutaneous administration dosage form (i.e., a dosage form for subcutaneous administration), and sometimes is an intramuscular administration dosage form (i.e., a dosage form for intramuscular administration). Certain implementations of a method of manufacture include loading a pharmaceutical composition into an auto-injector device (e.g., auto-injector pen device). A dosage form sometimes is a topical administration dosage form (i.e., a dosage form for topical administration).

### Certain Implementations

Following are non-limiting examples of certain implementations of the technology.

B0.1. A pharmaceutical composition, comprising an active ingredient and an oil component, wherein the active ingredient comprises a compound having a structure according to Formula A, a compound having a structure according to Formula B, a rimiducid analog (e.g., a compound having a structure according to Formula C, D, E, F or G, or a pharmaceutically acceptable salt, ester or amide thereof).

B0.2. The pharmaceutical composition of embodiment B0.1, wherein the active ingredient comprises a compound having a structure according to Formula A.

B0.3. The pharmaceutical composition of embodiment B0.2, wherein the active ingredient comprises a compound having a structure according to Formula B.

B0.4. The pharmaceutical composition of any one of embodiments B0.1-B0.3, wherein the active ingredient is at a concentration of about 1 millimolar (mM) to about 30 mM in the pharmaceutical composition.

B0.5. The pharmaceutical composition of any one of embodiments B0.1-B0.3, wherein the active ingredient is at a concentration of about 10 mM to about 20 mM in the pharmaceutical composition.

B0.6. The pharmaceutical composition of any one of embodiments B0.1-B0.3, wherein the active ingredient is at a concentration of about 12 mM to about 16 mM in the pharmaceutical composition.

B0.7. The pharmaceutical composition of any one of embodiments B0.1-B0.3, wherein the active ingredient is at a concentration of about 14 mM to about 15 mM in the pharmaceutical composition.

B1. The pharmaceutical composition of any one of embodiments B0.1-B0.7, wherein the oil component comprises one or more of medium-chain triglycerides (MCT), castor oil (CO), medium-chain triglycerides of caprylic (C8) and capric (C10) acids (CAP), oleoyl polyoxyl-6 glycerides (OPG), polyethoxylated castor oil (PECO), Triacetin, oleic acid (OA), propylene glycol monocaprylate (PGMC) (Type II), glycerol monooleate (GMO), caprylocaproyl polyoxyl-8 glycerides (CCPG), propylene glycol monolaurate (PGML) Type II, glyceryl monolinoleate (GML) and Pluronic® polymers (PP).

B2. The pharmaceutical composition of embodiment 1, wherein the oil component consists of one of MCT, CO, CAP, OPG, PECO, Triacetin, OA, PGMC, GMO, CCPG, PGML, GML or PP.

B3. The pharmaceutical composition of embodiment 1, wherein the oil component comprises two or more of MCT, CO, CAP, OPG, PECO, Triacetin, OA, PGMC, GMO, CCPG, PGML, GML and PP.

B4. The pharmaceutical composition of embodiment B3, wherein the oil component comprises a first component and a second component at a ratio between 95:5 and 5:95 by weight (w:w) for the oil component.

B5. The pharmaceutical composition of embodiment B4, wherein the oil component comprises MCT and CO at a ratio of about 60:40 to about 40:60.

B6. The pharmaceutical composition of embodiment B5, wherein the ratio is about 50:50.

B7. The pharmaceutical composition of embodiment B4, wherein the oil component comprises Triacetin and PECO at a ratio of about 30:70 to about 50:50.

B9. The pharmaceutical composition of embodiment B4, wherein the ratio is about 33:67.

B10. The pharmaceutical composition of any one of embodiments B1-B9, wherein the oil component comprises MCT.

B10.1. The pharmaceutical composition of any one of embodiments B1-B9, wherein the oil component consists of MCT or consists essentially of MCT.

E11. The pharmaceutical composition of any one of embodiments B1-B9, wherein the oil component comprises CO.

B11.1. The pharmaceutical composition of any one of embodiments B1-B9, wherein the oil component consists of CO or consists essentially of CO.

B12. The pharmaceutical composition of any one of embodiments B1-B9, wherein the oil component comprises CAP.

B13. The pharmaceutical composition of embodiment B12, wherein the oil component comprises Labrafac®.

B14. The pharmaceutical composition of any one of embodiments B1-B9, wherein the oil component comprises OPG.

B15. The pharmaceutical composition of embodiment B14, wherein the oil component comprises Labrafil®.

B16. The pharmaceutical composition of any one of embodiments B1-B9, wherein the oil component comprises PECO.

B17. The pharmaceutical composition of embodiment B16, wherein the oil component comprises Cremophor EL®.

B18. The pharmaceutical composition of any one of embodiments B1-B9, wherein the oil component comprises Triacetin.

B19. The pharmaceutical composition of any one of embodiments B1-B9, wherein the oil component comprises OA.

B20. The pharmaceutical composition of any one of embodiments B1-B9, wherein the oil component comprises PGMC.

B21. The pharmaceutical composition of embodiment B20, wherein the oil component comprises Capryol® 90.

B21.1. The pharmaceutical composition of any one of embodiments B1-B9, wherein the oil component comprises GMO.

B21.2. The pharmaceutical composition of embodiment B21.1, wherein the oil component comprises Peceol®).

B21.3. The pharmaceutical composition of any one of embodiments B1-B9, wherein the oil component comprises CCPG.

B21.4. The pharmaceutical composition of embodiment B21.3, wherein the oil component comprises Labrasol®.

B21.5. The pharmaceutical composition of any one of embodiments B1-B9, wherein the oil component comprises PGML Type II.

B21.6. The pharmaceutical composition of embodiment B21.5, wherein the oil component comprises Lauroglycol™ 90.

B21.7. The pharmaceutical composition of any one of embodiments B1-B9, wherein the oil component comprises GML.

B21.8. The pharmaceutical composition of embodiment B21.7, wherein the oil component comprises Maisine® CC.

B21.9. The pharmaceutical composition of any one of embodiments B0.01-B9, wherein the oil component comprises PP.

B22. The pharmaceutical composition of any one of embodiments B0.1-B21.9, wherein the oil component is about 50% to about 100% by volume in the pharmaceutical composition.

B22.1. The pharmaceutical composition of embodiment B22, wherein the oil component is about 60% to about 100% by volume in the pharmaceutical composition.

B23. The pharmaceutical composition of embodiment B22, wherein the oil component is about 70% to about 100% by volume in the pharmaceutical composition.

B24. The pharmaceutical composition of embodiment B22, wherein the oil component is about 80% to about 100% by volume in the pharmaceutical composition.

B25. The pharmaceutical composition of embodiment B22, wherein the oil component is about 90% to about 100% by volume in the pharmaceutical composition.

B26. The pharmaceutical composition of embodiment B22, wherein the oil component is about 95% to about 100% by volume in the pharmaceutical composition.

B27. The pharmaceutical composition of any one of embodiments B22-B26, wherein the oil component consists of or consists essentially of MCT.

B28. The pharmaceutical composition of embodiment B27, wherein the composition contains about 1170 mM to about 2323 mM MCT.

B29. The pharmaceutical composition of embodiment B27, wherein the composition contains about 1700 mM to about 2323 mM MCT.

B30. The pharmaceutical composition of embodiment B27, wherein the composition contains about 2000 mM to about 2323 mM MCT.

B31. The pharmaceutical composition of embodiment B27, wherein the composition contains about 2200 mM to about 2323 mM MCT.

B32. The pharmaceutical composition of any one of embodiments B22-B26, wherein the oil component consists of or consists essentially of CO.

B33. The pharmaceutical composition of embodiment B32, wherein the composition contains about 600 mM to about 1023 mM CO.

B34. The pharmaceutical composition of embodiment B32, wherein the composition contains about 900 mM to about 1023 mM CO.

B35. The pharmaceutical composition of embodiment B32, wherein the composition contains about 1000 mM to about 1023 mM CO.

B36. The pharmaceutical composition of any one of embodiments B22-B35, comprising a surfactant component.

B37. The pharmaceutical composition of embodiment B36, wherein the composition contains about 0.1% to about 35% of the surfactant by volume.

B38. The pharmaceutical composition of embodiment B36, wherein the composition contains about 5% to about 35% of the surfactant by volume.

B39. The pharmaceutical composition of embodiment B36, wherein the composition contains about 10% to about 30% of the surfactant by volume.

B40. The pharmaceutical composition of any one of embodiments B36-B39, wherein the surfactant consists of or consists essentially of PS80.

B41. The pharmaceutical composition of any one of embodiments B36-B40, wherein the composition contains about 5 mM to about 330 mM PS80.

B42. The pharmaceutical composition of embodiment B41, wherein the composition contains about 80 mM to about 250 mM PS80.

B43. The pharmaceutical composition of embodiment B41, wherein the composition contains about 120 mM to about 240 mM PS80.

B44. The pharmaceutical composition of any one of embodiments B0.1-B43, comprising an aqueous component, a surfactant component and an oil component.

B45. A pharmaceutical composition of any one of embodiments B0.1-B43, comprising an aqueous component, a solvent component, a surfactant component and an oil component.

B46. The pharmaceutical composition of any one of embodiments B0.1-B45 which composition is an oil-in-water emulsion comprising particles.

B47. The pharmaceutical composition of embodiment B46, wherein the particles in the composition are defined by a mean particle diameter of 200 nm or less.

B48. The pharmaceutical composition of embodiment B46, wherein the particles in the composition are defined by a mean particle diameter of 100 nm or less.

B49. The pharmaceutical composition of embodiment B47 or B48, wherein a standard deviation of the mean particle diameter is about 20 nm or less.

B50. The pharmaceutical composition of embodiment B47 or B48, wherein a standard deviation of the mean particle diameter is about 10 nm or less.

B51. The pharmaceutical composition of embodiment B47 or B48, wherein a standard deviation of the mean particle diameter is about 5 nm or less.

B52. The pharmaceutical composition of embodiment B47 or B48, wherein a standard deviation of the mean particle diameter is about 1 nm or less.

B53. The pharmaceutical composition of any one of embodiments B46-B52, wherein the particles in the composition are defined by a dispersity of about 0.005 to about 0.30.

B54. The pharmaceutical composition of any one of embodiments B46-B52, wherein the particles in the composition are defined by a dispersity of about 0.10 to about 0.30.

B55. The pharmaceutical composition of any one of embodiments B46-B52, wherein the particles in the composition are defined by a dispersity of about 0.10 to about 0.25.

B56. The pharmaceutical composition of any one of embodiments B53-B55, wherein a standard deviation of the dispersity of about 0.10 or less.

B57. The pharmaceutical composition of any one of embodiments B53-B55, wherein a standard deviation of the dispersity of about 0.05 or less.

B58. The pharmaceutical composition of any one of embodiments B53-B55, wherein a standard deviation of the dispersity of about 0.01 or less.

B59. A pharmaceutical composition, comprising an active ingredient, an aqueous component, a solvent component and a surfactant component, wherein the active ingredient comprises a compound having a structure according to Formula A, a compound having a structure according to Formula B, a rimiducid analog (e.g., a compound having a structure according to Formula C, D, E, F or G, or a pharmaceutically acceptable salt, ester or amide thereof).

B60. The pharmaceutical composition of any one of embodiments B44-B59, wherein the aqueous component comprises water or saline.

B61. The pharmaceutical composition of embodiment B60, wherein the aqueous component consists of water or consists essentially of water.

B62. The pharmaceutical composition of any one of embodiments B44-B61, wherein the surfactant component comprises a solubility enhancer component.

B63. The pharmaceutical composition of embodiment B62, wherein the surfactant component is a solubility enhancer component comprising one or more of lecithin; polysorbate (PS) 20, 40, 60 and/or 80; Span 20, 40, 60 and/or 80; Pluronic® polymers (PP), propylene glycol (PG) and cyclodextrin (CD).

B64. The pharmaceutical composition of embodiment B62, wherein the solubility enhancer component comprises a first component and a second component at a ratio between 95:5 and 5:95 by weight (w:w) for the solubility enhancer component.

B65. The pharmaceutical composition of embodiment B64, wherein the solubility enhancer component comprises the first component and the second component at a ratio of about 60:40 to about 40:60.

B66. The pharmaceutical composition of any one of embodiments B62-B65, wherein the solubility enhancer component comprises lecithin.

B67. The pharmaceutical composition of any one of embodiments B62-B66, wherein the surfactant component or the solubility enhancer component comprises PS 20, 40, 60 and/or 80.

B68. The pharmaceutical composition of embodiment B67, wherein the surfactant component or the solubility enhancer component comprises PS80.

B69. The pharmaceutical composition of embodiment B67, wherein the surfactant component or the solubility enhancer component consists of PS80 or consists essentially of PS80.

B70. The pharmaceutical composition of any one of embodiments B62-B69, wherein the solubility enhancer component comprises Span 20, 40, 60 and/or 80.

B71. The pharmaceutical composition of any one of embodiments B62-B70, wherein the solubility enhancer component comprises PP.

B72. The pharmaceutical composition of any one of embodiments B62-B71, wherein the solubility enhancer component comprises PG.

B73. The pharmaceutical composition of any one of embodiments B62-B72, wherein the solubility enhancer component comprises CD.

B74. The pharmaceutical composition of any one of embodiments B0.1-B73, comprising a penetration enhancer component.

B75. The pharmaceutical composition of embodiment B74, wherein the penetration enhancer component comprises one or more of ethanol and/or 2-(2-ethoxyethoxy)ethanol (EEE).

B76. The pharmaceutical composition of embodiment B74, wherein the penetration enhancer component comprises one or both of Transcutol® and Transcutol® HP.

B77. The pharmaceutical composition of any one of embodiments B44-B35, wherein the solvent component is a polyethylene glycol (PEG).

B78. The pharmaceutical composition of embodiment B77, wherein the PEG comprises PEG400.

B79. The pharmaceutical composition of any one of embodiments B0.1-B78, comprising an oil component, aqueous component and one or more of a solubility enhancer component and a penetration enhancer component.

B80. The pharmaceutical composition of embodiment B79, comprising MCT:water:PS80:Span80:ethanol.

B81. The pharmaceutical composition of embodiment B80, comprising MCT:water:PS80:Span80:ethanol (25-35:45-55:5-10:5-10:1-10).

B82. The pharmaceutical composition of embodiment B81, comprising MCT:water:PS80:Span80:ethanol (30:50:7.5:7.5:5).

B83. The pharmaceutical composition of embodiment B79, comprising CO:saline:PS60:EEE.

B84. The pharmaceutical composition of embodiment B83, comprising CO:saline:PS60:EEE (15-25:60-70:5-15:1-10).

B85. The pharmaceutical composition of embodiment B83, comprising CO:saline:PS60:EEE (20:65:10:5).

B86. The pharmaceutical composition of embodiment B79, comprising CO:saline:PS80

B87. The pharmaceutical composition of embodiment B86, comprising CO:saline:PS80 (10-20:65-75:10-20).

B88. The pharmaceutical composition of embodiment B86, comprising CO:saline:PS80 (15:70:15).

B89. The pharmaceutical composition of embodiment B79, comprising OA:saline:Span40:EEE.

B90. The pharmaceutical composition of embodiment B89, comprising OA:saline:Span40:EEE (30-40:45-55:5-15:1-10).

B91. The pharmaceutical composition of embodiment B89, comprising OA:saline:Span40:EEE (35:50:10:5).

B92. The pharmaceutical composition of embodiment B79, comprising PECO:saline:Span80.

B93. The pharmaceutical composition of embodiment B92, comprising PECO:saline:Span80 (10-20:70-80:5-15).

B94. The pharmaceutical composition of embodiment B92, comprising PECO:saline:Span80 (15:75:10).

B95. The pharmaceutical composition of embodiment B79, comprising CAP:saline:lecithin.

B96. The pharmaceutical composition of embodiment B95, comprising CAP:saline:lecithin (15-25:45-55:25-35).

B97. The pharmaceutical composition of embodiment B95, comprising CAP:saline:lecithin (20:50:30).

B98. The pharmaceutical composition of embodiment B79, comprising OPG:saline:CD:EEE.

B99. The pharmaceutical composition of embodiment B98, comprising OPG:saline:CD:EEE (10-20:60-70:10-20:1-10).

B100. The pharmaceutical composition of embodiment B98, comprising OPG:saline:CD:EEE (15:65:15:5).

B101. The pharmaceutical composition of embodiment B79, comprising PGM:saline:PP:ethanol.

B102. The pharmaceutical composition of embodiment B101, comprising PGM:saline:PP:ethanol (25-35:45-55:10-20:1-10).

B103. The pharmaceutical composition of embodiment B101, comprising PGM:saline:PP:ethanol (30:50:15:5).

B104. A pharmaceutical composition, comprising: an active ingredient comprising a compound having a structure according to Formula A, a compound having a structure according to Formula B; an aqueous component including water; a surfactant component including a polysorbate (PS); and an oil component including castor oil (CO).

B105. The pharmaceutical composition of embodiment B104, wherein the PS comprises PS80.

B106. The pharmaceutical composition of embodiment B104 or B105, wherein the active ingredient consists of or consists essentially of a compound having a structure according to Formula A or Formula B.

B107. The pharmaceutical composition of any one of embodiments B104-B106, wherein the composition comprises about 5 mM to about 9 mM of a compound having the structure according to Formula A or Formula B, or about 10 mM to about 20 mM of a compound having the structure according to Formula A or Formula B.

B108. The pharmaceutical composition of any one of embodiments B104-B107, wherein the composition comprises about 6.9 mM to about 7.3 mM of a compound having the structure according to Formula A or Formula B or about 13 mM to about 15 mM of a compound having the structure according to Formula A or Formula B.

B109. A pharmaceutical composition, comprising: an active ingredient consisting of or consisting essentially of a compound having a structure according to Formula A or Formula B; an aqueous component consisting of or consisting essentially of water, a surfactant component consisting of or consisting essentially of PS80, and an oil component consisting of or consisting essentially of CO.

B110. The pharmaceutical composition of any one of embodiments B104-B109, wherein the aqueous component is at a percent volume of about 50% to about 70%, the oil component is at a percent volume of about 20% to about 40%, and the surfactant component is at a percent volume of about 1% to about 20%.

B111. The pharmaceutical composition of any one of embodiments B104-B110, wherein the aqueous component, the oil component and the surfactant component each are at a percent volume and the percent volumes of the aqueous component, the oil component and the surfactant component sum to 100%; the percent volume of the aqueous component is about 55% to about 65%; the percent volume of the oil component is about 25% to about 35%; and the percent volume of the surfactant component is about 5% to about 15%.

B112. The pharmaceutical composition of any one of embodiments B104-B111, wherein the percent volume of the aqueous component is about 60%, the percent volume of the oil component is about 30%, and the percent volume of the surfactant component is about 10%.

B113. The pharmaceutical composition of any one of embodiments B104-B112, wherein the composition comprises: about 8 mg to about 12 mg per mL volume, or about 15 mg to about 25 mg per mL volume, of a compound having a structure according to Formula A or Formula B; about 50% to about 70% water by volume; about 20% to about 40% CO by volume; and about 1% to about 20% PS80 by volume; wherein the percentage by volume of the water, the percentage by volume of the CO and the percentage by volume of the PS80 sum to 100%.

B114. The pharmaceutical composition of any one of embodiments B104-B113, wherein the composition comprises: about 8 mg to about 12 mg per mL volume, or about 15 mg to about 25 mg per mL volume, of a compound having a structure according to Formula A or Formula B; about 55% to about 65% water by volume; about 25% to about 35% CO by volume; and about 5% to about 15% PS80 by volume, wherein the percentage by volume of the water; the percentage by volume of the CO and the percentage by volume of the PS80 sum to 100%.

B115. The pharmaceutical composition of any one of embodiments B104-B114, wherein the composition comprises: about 5 mM to about 9 mM or about 10 mM to about 20 mM of a compound having the structure of Formula A or Formula B; about 100 mM to about 450 mM CO; about 40 mM to about 125 mM PS80; and about 55% to about 65% water by volume.

B116. The pharmaceutical composition of any one of embodiments B104-B115, wherein the composition comprises: about 10 mg per mL volume or about 20 mg per mL volume of a compound having a structure according to Formula B; about 60% water by volume, about 30% CO by volume, and about 10% PS80 by volume.

B117. The pharmaceutical composition of any one of embodiments B104-B116, wherein the composition comprises: about 6.9 mM to about 7.3 mM or about 13 mM to about 15 mM of a compound having the structure of Formula B; about 305 mM to about 310 mM CO; about 79 mM to about 83 mM PS80; and about 60% water by volume.

B118. The pharmaceutical composition of any one of embodiments B104-B117, wherein the pharmaceutical composition comprises particles.

B119. The pharmaceutical composition of any one of embodiments B104-B118, wherein the particles have one or more of the following characteristics: (i) a mean particle diameter of less than or equal to 100 nm; (ii) a standard deviation of the mean particle diameter of about 20 nm or less; (iii) a dispersity of about 0.10 to about 0.25; and (iv) a standard deviation of the dispersity of about 0.10 or less.

B120. A pharmaceutical composition, comprising: an active ingredient comprising a compound having a structure according to Formula A, a compound having a structure according to Formula B; an aqueous component comprising water; a surfactant component comprising a polysorbate (PS); and an oil component comprising Medium Chain Triglycerides (MCT).

B121. The pharmaceutical composition embodiment B120, wherein the PS comprises PS80.

B122. The pharmaceutical composition of embodiment B120 or B121, wherein the MCT comprises one or more of C6, C8, C10 and C12 fatty acids.

B123. The pharmaceutical composition of embodiment B122, wherein one or more or all of the C6, C8, C10 and C12 fatty acids are saturated fatty acids.

B124. The pharmaceutical composition of embodiment B122 or B123, wherein the MCT comprises a C8 fatty acid and a C10 fatty acid and optionally a C12 fatty acid.

B125. The pharmaceutical composition of any one of embodiments B120-B124, wherein the MCT is from a plant.

B126. The pharmaceutical composition of embodiment B125, wherein the MCT is from coconut.

B127. The pharmaceutical composition of any one of embodiments B120-B126, wherein the active ingredient consists of or consists essentially of a compound having a structure according to Formula A or Formula B.

B128. The pharmaceutical composition of any one of embodiments B120-B127, wherein the composition comprises about 5 mM to about 9 mM or about 10 mM to about 20 mM of a compound having the structure according to Formula A or Formula B.

B129. The pharmaceutical composition of any one of embodiments B120-128, wherein the composition comprises about 6.9 mM to about 7.3 mM or about 13 mM to about 15 mM of a compound having the structure according to Formula A or Formula B.

B130. A pharmaceutical composition comprising: an active ingredient consisting of or consisting essentially of a compound having a structure according to Formula A or Formula B; an aqueous component consisting of or consisting essentially of water, a surfactant component consisting of or consisting essentially of PS80, and an oil component consisting of or consisting essentially of MCT.

B131. The pharmaceutical composition of any one of embodiments B120-B130, wherein the aqueous component is at a percent volume of about 50% to about 70%, the oil component is at a percent volume of about 20% to about 40%, and the surfactant component is at a percent volume of about 1% to about 20%.

B131. The pharmaceutical composition of any one of embodiments B120-B130, wherein the aqueous component, the oil component and the surfactant component each are at a percent volume and the percent volumes of the aqueous component, the oil component and the surfactant component sum to 100%; the percent volume of the aqueous component is about 55% to about 65%; the percent volume of the oil component is about 25% to about 35%; and the percent volume of the surfactant component is about 5% to about 15%.

B132. The pharmaceutical composition of any one of embodiments B120-B130, wherein the percent volume of the aqueous component is about 60%, the percent volume of the oil component is about 30%, and the percent volume of the surfactant component is about 10%.

B133. The pharmaceutical composition of any one of embodiments B120-B130, wherein the pharmaceutical composition comprises: about 8 mg to about 12 mg per mL volume, or about 15 mg to about 25 mg per mL volume, of a compound having the structure of Formula A or Formula B, about 50% to about 70% water by volume, about 20% to about 40% MCT by volume, and about 1% to about 20% PS80 by volume, wherein the percentage by volume of the water, the percentage by volume of the MCT and the percentage by volume of the PS80 sum to 100%.

B134. The pharmaceutical composition of any one of embodiments B120-B130, wherein the pharmaceutical composition comprises: about 8 mg to about 12 mg per mL volume, or about 15 mg to about 25 mg per mL volume, of a compound having the structure of Formula A or Formula B, about 55% to about 65% water by volume, about 25% to about 35% MCT by volume, and about 5% to about 15% PS80 by volume, wherein the percentage by volume of the water, the percentage by volume of the MCT and the percentage by volume of the PS80 sum to 100%.

B134.1. The pharmaceutical composition of any one of embodiments B120-B130, wherein the pharmaceutical composition comprises: about 8 mg to about 12 mg per mL volume, or about 15 mg to about 25 mg per mL volume, of a compound having the structure of Formula B, about 60% water by volume, about 30% MCT by volume, and about 10% PS80 by volume, wherein the percentage by volume of the water, the percentage by volume of the MCT and the percentage by volume of the PS80 sum to 100%.

B135. The pharmaceutical composition of any one of embodiments B120-B130, wherein the pharmaceutical composition comprises: about 5 mM to about 9 mM or about 10 mM to about 20 mM of a compound having the structure of Formula A or Formula B; about 110 mM to about 1170 mM MCT; about 40 mM to about 125 mM PS80; and about 55% to about 65% water by volume.

B136. The pharmaceutical composition of any one of embodiments B120-B130, wherein the pharmaceutical composition comprises: about 6.5 mM to about 7.5 mM or about 12 mM to about 16 mM of a compound having the structure of Formula A or Formula B; about 500 mM to about 900 mM MCT; about 60 mM to about 100 mM PS80; and about 55% to about 65% water by volume.

B137. The pharmaceutical composition of any one of embodiments B120-B130, wherein the pharmaceutical composition comprises: about 6.9 mM to about 7.3 mM or about 13 mM to about 15 mM of a compound having the structure of Formula A or Formula B; about 695 mM to about 700 mM MCT; about 79 mM to about 83 mM PS80; and about 60% water by volume.

B138. The pharmaceutical composition of any one of embodiments B120-B137, wherein the pharmaceutical composition comprises particles.

B139. The pharmaceutical composition of embodiment B138, wherein the particles have one or more of the following characteristics: (i) a mean particle diameter of less than or equal to 100 nm; (ii) a standard deviation of the mean particle diameter of about 20 nm or less; (iii) a dispersity of about 0.10 to about 0.25; and (iv) a standard deviation of the dispersity of about 0.10 or less.

B140. A pharmaceutical composition, comprising: an active ingredient comprising a compound having a structure according to Formula A, a compound having a structure according to Formula B; an aqueous component comprising water; a surfactant component comprising a polysorbate (PS); a solvent component comprising a polyethylene glycol (PEG); and an oil component comprising Medium Chain Triglycerides (MCT).

B141. The pharmaceutical composition embodiment B140, wherein the PS comprises PS80.

B142. The pharmaceutical composition of embodiment B140 or B141, wherein the MCT comprises one or more of C6, C8, C10 and C12 fatty acids.

B143. The pharmaceutical composition of embodiment B142, wherein one or more or all of the C6, C8, C10 and C12 fatty acids are saturated fatty acids.

B144. The pharmaceutical composition of embodiment B142 or B143, wherein the MCT comprises a C8 fatty acid and a C10 fatty acid and optionally a C12 fatty acid.

B145. The pharmaceutical composition of any one of embodiments B140-B144, wherein the MCT is from a plant.

B146. The pharmaceutical composition of embodiment B145, wherein the MCT is from coconut.

B147. The pharmaceutical composition of any one of embodiments B140-B146, wherein the PEG comprises PEG400.

B148. The pharmaceutical composition of any one of embodiments B140-B147, wherein the active ingredient consists of or consists essentially of a compound having a structure according to Formula A or Formula B.

B149. The pharmaceutical composition of any one of embodiments B140-B148, wherein the composition comprises about 5 mM to about 9 mM or about 10 mM to about 20 mM of a compound having the structure according to Formula A or Formula B.

B150. The pharmaceutical composition of any one of embodiments B140-149, wherein the composition comprises about 6.9 mM to about 7.3 mM or about 13 mM to about 15 mM of a compound having the structure according to Formula A or Formula B.

B151. The pharmaceutical composition of any one of embodiments B140-150, wherein the composition comprises: an active ingredient consisting of or consisting essentially of a compound having a structure according to Formula A or Formula B; an aqueous component consisting of or consisting essentially of water; a surfactant component consisting of or consisting essentially of PS80; a solvent component consisting of or consisting essentially of PEG400; and an oil component consisting of or consisting essentially of MCT.

B152. The pharmaceutical composition of any one of embodiments B140-B151, wherein the aqueous component is at a percent volume of about 30% to about 50%, the oil component is at a percent volume of about 20% to about 40%, the solvent component is at a percent volume of about 5% to about 25%, and the surfactant component is at a percent volume of about 5% to about 25%.

B153. The pharmaceutical composition of any one of embodiments B140-B151, wherein the aqueous component, the solvent component, the oil component and the surfactant component each are at a percent volume and the percent volumes of the aqueous component, the solvent component, the oil component and the surfactant component sum to 100%; the percent volume of the aqueous component is about 35% to about 45%; the percent volume of the oil component is about 25% to about 35%; the percent volume of the solvent component is about 10% to about 20%; and the percent volume of the surfactant component is about 10% to about 20%.

B154. The pharmaceutical composition of any one of embodiments B140-B151, wherein the percent volume of the aqueous component is about 40%, the percent volume of the oil component is about 30%, the percent volume of the solvent component is about 15%, and the percent volume of the surfactant component is about 15%.

B155. The pharmaceutical composition of any one of embodiments B140-B151, wherein the composition comprises: about 8 mg to about 12 mg per mL volume, or about 15 mg to about 25 mg per mL volume, of a compound having the structure of Formula A or Formula B; about 30% to about 50% water by volume; about 20% to about 40% MCT by volume; about 5% to about 25% PEG400 by volume; and about 5% to about 25% PS80 by volume, wherein the percentage by volume of the water, the percentage by volume of the MCT, the percentage by volume of the PEG400 and the percentage by volume of the PS80 sum to 100%.

B156. The pharmaceutical composition of any one of embodiments B140-B151, wherein the pharmaceutical composition comprises: about 8 mg to about 12 mg per mL volume, or about 15 mg to about 25 mg per mL volume, of a compound having the structure of Formula A or Formula B; about 35% to about 45% water by volume; about 25% to about 35% MCT by volume; about 10% to about 20% PEG400 by volume; and about 10% to about 20% PS80 by volume, where the percentage by volume of the water, the percentage by volume of the MCT and the percentage by volume of the PS80 sum to 100%.

B157. The pharmaceutical composition of any one of embodiments B140-B151, wherein the pharmaceutical composition comprises: about 10 mg per mL volume or about 20 mg per mL volume of a compound having the structure of Formula B; about 40% water by volume, about 30% MCT by volume; about 15% PEG400 by volume; and about 15% PS80 by volume.

B158. The pharmaceutical composition of any one of embodiments B140-B151, wherein the pharmaceutical composition comprises: about 5 mM to about 9 mM or about 10 mM to about 20 mM of a compound having the structure of Formula A or Formula B; about 110 mM to about 1170 mM MCT; about 80 mM to about 165 mM PS80; about 280 mM to about 570 mM PEG400; and about 35% to about 45% water by volume.

B159. The pharmaceutical composition of any one of embodiments B140-B151, wherein the pharmaceutical composition comprises: about 6 mM to about 8 mM or about 12 mM to about 16 mM of a compound having the structure of Formula A or Formula B; about 500 mM to about 900 mM MCT; about 90 mM to about 150 mM PS80; about 400 mM to about 450 mM PEG400; and about 35% to about 45% water by volume.

B160. The pharmaceutical composition of any one of embodiments B140-B151, wherein the pharmaceutical composition comprises: about 6.9 mM to about 7.3 mM or about 13 mM to about 15 mM of a compound having the structure of Formula A or Formula B; about 695 mM to about 700 mM MCT; about 119 mM to about 123 mM PS80; about 422 mM to about 426 mM PEG400; and sometimes about 40% water by volume.

B161. The pharmaceutical composition of any one of embodiments B140-B160, wherein the pharmaceutical composition comprises particles.

B162. The pharmaceutical composition of embodiments B161, wherein the particles have one or more of the following characteristics: (i) a mean particle diameter of less than or equal to 100 nm; (ii) a standard deviation of the mean particle diameter of about 20 nm or less; (iii) a dispersity of about 0.10 to about 0.25; and (iv) a standard deviation of the dispersity of about 0.10 or less.

B163. A pharmaceutical composition, comprising: an active ingredient comprising a compound having a structure according to Formula A, a compound having a structure according to Formula B; an aqueous component comprising water; a surfactant component comprising a polysorbate (PS); a solvent component comprising a polyethylene glycol (PEG); and an oil component comprising castor oil (CO).

B164. The pharmaceutical composition of embodiment B163, wherein the PS comprises PS80.

B165. The pharmaceutical composition of embodiment B163 or B164, wherein PEG comprises PEG400.

B166. The pharmaceutical composition of any one of embodiments B163-B165, wherein the active ingredient consists of or consists essentially of a compound having a structure according to Formula A or Formula B.

B167. The pharmaceutical composition of any one of embodiments B163-B166, wherein the composition comprises about 5 mM to about 9 mM or about 10 mM to about 20 mM of a compound having the structure according to Formula A or Formula B.

B168. The pharmaceutical composition of any one of embodiments B163-B167, wherein the composition comprises about 6.9 mM to about 7.3 mM or about 13 mM to about 15 mM of a compound having the structure according to Formula A or Formula B.

B169. The pharmaceutical composition of any one of embodiments B163-B168, wherein the active ingredient consists of or consists essentially of a compound having a structure according to Formula A or Formula B.

B170. The pharmaceutical composition of any one of embodiments B163-B169, wherein the composition comprises: an active ingredient consisting of or consisting essentially of a compound having the structure of Formula A or Formula B; an aqueous component consisting of or consisting essentially of water; a surfactant component consisting of or consisting essentially of PS80; a solvent component consisting of or consisting essentially of PEG400; and an oil component consisting of or consisting essentially of CO.

B171. The pharmaceutical composition of any one of embodiments B163-B169, wherein the aqueous component is at a percent volume of about 30% to about 50%, the oil component is at a percent volume of about 20% to about 40%, the solvent component is at a percent volume of about 5% to about 25%, and the surfactant component is at a percent volume of about 5% to about 25%.

B172. The pharmaceutical composition of any one of embodiments B163-B169, wherein the aqueous component, the solvent component, the oil component and the surfactant component each are at a percent volume and the percent volumes of the aqueous component, the solvent component, the oil component and the surfactant component sum to 100%; the percent volume of the aqueous component is about 35% to about 45%; the percent volume of the oil component is about 25% to about 35%; the percent volume of the solvent component is about 10% to about 20%; and the percent volume of the surfactant component is about 10% to about 20%.

B173. The pharmaceutical composition of any one of embodiments B163-B169, wherein the percent volume of the aqueous component is about 40%, the percent volume of the oil component is about 30%, the percent volume of the solvent component is about 15%, and the percent volume of the surfactant component is about 15%.

B174. The pharmaceutical composition of any one of embodiments B163-B169, wherein composition comprises: about 8 mg to about 12 mg per mL volume, or about 15 mg to about 25 mg per mL volume, of a compound having the structure of Formula A or Formula B; about 30% to about 50% water by volume; about 20% to about 40% CO by volume; about 5% to about 25% PEG400 by volume; and about 5% to about 25% PS80 by volume, wherein the percentage by volume of the water, the percentage by volume of the CO, the percentage by volume of the PEG400 and the percentage by volume of the PS80 sum to 100%.

B175. The pharmaceutical composition of any one of embodiments B163-B169, wherein the composition comprises: about 8 mg to about 12 mg per mL volume, or about 15 mg to about 25 mg per mL volume, of a compound having the structure of Formula A or Formula B; about 35% to about 45% water by volume; about 25% to about 35% CO by volume; about 10% to about 20% PEG400 by volume; and about 10% to about 20% PS80 by volume, where the percentage by volume of the water, the percentage by volume of the CO and the percentage by volume of the PS80 sum to 100%.

B175. The pharmaceutical composition of any one of embodiments B163-B169, wherein the composition comprises: about 10 mg per mL volume or about 20 mg per mL volume of a compound having the structure of Formula B; about 40% water by volume; about 30% CO by volume; about 15% PEG400 by volume; and about 15% PS80 by volume.

B176. The pharmaceutical composition of any one of embodiments B163-B169, wherein the composition comprises: about 5 mM to about 9 mM or about 10 mM to about 20 mM of a compound having the structure of Formula A or Formula B; about 50 mM to about 520 mM CO; about 80 mM to about 165 mM PS80; about 280 mM to about 570 mM PEG400; and about 35% to about 45% water by volume.

B177. The pharmaceutical composition of any one of embodiments B163-B169, wherein the composition comprises: about 6 mM to about 8 mM or about 12 mM to about 16 mM of a compound having the structure of Formula A or Formula B; about 200 mM to about 400 mM CO; about 90 mM to about 150 mM PS80; about 370 mM to about 470 mM PEG400; and about 35% to about 45% water by volume.

B178. The pharmaceutical composition of any one of embodiments B163-B169, wherein the composition comprises: about 6.9 mM to about 7.3 mM or about 13 mM to about 15 mM of a compound having the structure of Formula B; about 305 mM to about 310 mM CO; about 119 mM to about 123 mM PS80; about 422 mM to about 426 mM PEG400; and about 40% water by volume.

B179. The pharmaceutical composition of any one of embodiments B163-B178, wherein the pharmaceutical composition comprises particles.

B180. The pharmaceutical composition of embodiments B179, wherein the particles have one or more of the following characteristics: (i) a mean particle diameter of less than or equal to 100 nm; (ii) a standard deviation of the mean particle diameter of about 20 nm or less; (iii) a dispersity of about 0.10 to about 0.25; and (iv) a standard deviation of the dispersity of about 0.10 or less.

B181. A pharmaceutical composition, comprises: an active ingredient comprising a compound having a structure according to Formula A, a compound having a structure according to Formula B; an aqueous component comprising water; a solvent component comprising a polyethylene glycol (PEG); and a surfactant component comprising a polysorbate (PS).

B182. The pharmaceutical composition of embodiment B181, wherein the PEG comprises PEG400.

B183. The pharmaceutical composition of embodiment B181 or B182, wherein the PS comprises PS80.

B184. The pharmaceutical composition of any one of embodiments B181-B183, wherein the active ingredient consists of or consists essentially of a compound having a structure according to Formula A or Formula B.

B185. The pharmaceutical composition of any one of embodiments B181-B184, wherein the composition comprises about 5 mM to about 9 mM or about 10 mM to about 20 mM of a compound having the structure according to Formula A or Formula B.

B186. The pharmaceutical composition of any one of embodiments B181-B185, wherein the composition comprises about 6.9 mM to about 7.3 mM or about 13 mM to about 15 mM of a compound having the structure according to Formula A or Formula B.

B187. The pharmaceutical composition of any one of embodiments B181-B186, wherein composition comprises: an active ingredient consisting of or consisting essentially of a compound having a structure according to Formula A, a compound having a structure according to Formula B; an aqueous component consisting of or consisting essentially of water; a solvent component consisting of or consisting essentially of PEG400; and a surfactant component consisting of or consisting essentially of PS80.

B188. The pharmaceutical composition of any one of embodiments B181-B186, wherein the aqueous component is at a percent volume of about 30% to about 50%, the solvent component is at a percent volume of about 20% to about 40%, and the surfactant component is at a percent volume of about 20% to about 40%.

B189. The pharmaceutical composition of any one of embodiments B181-B186, wherein the aqueous component, the solvent component and the surfactant component each are at a percent volume and the percent volumes of the aqueous component, the solvent component and the surfactant component sum to 100%, the percent volume of the aqueous component is about 35% to about 45%, the percent volume of the solvent component is about 25% to about 35%, and the percent volume of the surfactant component is about 25% to about 35%.

B190. The pharmaceutical composition of any one of embodiments B181-B186, wherein the percent volume of the aqueous component is about 40%, the percent volume of the solvent component is about 30%, and the percent volume of the surfactant component is about 30%.

B191. The pharmaceutical composition of any one of embodiments B181-B186, wherein the composition comprises about 8 mg to about 12 mg per mL volume, or about 15 mg to about 25 mg per mL volume, of a compound having the structure of Formula A or Formula B; about 30% to about 50% water by volume; about 20% to about 40% PEG400 by volume; and about 20% to about 40% PS80 by volume, where the percentage by volume of the water, the percentage by volume of the PEG400 and the percentage by volume of the PS80 sum to 100%.

B192. The pharmaceutical composition of any one of embodiments B181-B186, wherein the composition comprises about 10 mg per mL volume, or about 20 mg per mL volume, of a compound having the structure of Formula B; about 40% water by volume; about 30% PEG400 by volume; and about 30% PS80 by volume.

B193. The pharmaceutical composition of any one of embodiments B181-B186, wherein the composition comprises about 5 mM to about 9 mM or about 10 mM to about 20 mM of a compound having the structure of Formula A or Formula B; about 550 mM to about 1150 mM PEG400; about 160 mM to about 330 mM PS80, and about 30% to about 50% water by volume.

B194. The pharmaceutical composition of any one of embodiments B181-B186, wherein the composition comprises about 6 mM to about 8 mM or about 12 mM to about 16 mM of a compound having the structure of Formula A or Formula B; about 700 mM to about 950 mM PEG400; about 220 mM to about 280 mM PS80, and about 35% to about 45% water by volume.

B195. The pharmaceutical composition of any one of embodiments B181-B186, wherein the composition comprises about 6.9 mM to about 7.3 mM or about 13 mM to about 15 mM of a compound having the structure of Formula B; about 830 mM to about 870 mM PEG400; and about 240 mM to about 245 mM PS80, and about 40% water by volume.

B196. A pharmaceutical composition, comprising an active ingredient and a polar solvent component, wherein the active ingredient comprises a compound having a structure according to Formula A, a compound having a structure according to Formula B, or a rimiducid analog.

B197. The pharmaceutical composition of embodiment B196, comprising no added water.

B198. The pharmaceutical composition of embodiment B196 or B197, comprising no added oil component.

B199. The pharmaceutical composition of any one of embodiments B196-B198, consisting of or consisting essentially of the active ingredient and the polar solvent component.

B200. The pharmaceutical composition of any one of embodiments B196-B199, comprising about 5 milligrams per gram (mg/g) to about 200 mg/g, weight to weight, of a compound having a structure according to Formula A or Formula B.

B201. The pharmaceutical composition of embodiment B200, comprising about 20 mg/g or greater, weight to weight, of a compound having a structure according to Formula A or Formula B.

B202. The pharmaceutical composition of any one of embodiments B196-B200, comprising about 5 mM to about 140 mM of a compound having the structure of Formula A or Formula B.

B203. The pharmaceutical composition of embodiment B202, comprising about 28 mM or greater of a compound having a structure according to Formula A or Formula B.

B204. The pharmaceutical composition of any one of embodiments B196-B203, wherein the polar solvent component comprises one or more of N-methyl 2-pyrrolidone (NMP), polyethylene glycol 300 (PEG300), polyethylene glycol 400 (PEG400), dimethyl sulfoxide (DMSO), 2-(2-ethoxyethoxy)ethanol (EEE) or ethanol.

B205. The pharmaceutical composition of embodiment B204, wherein the EEE is Transcutol®.

B206. The pharmaceutical composition of any one of embodiments B196-B205, wherein the polar solvent component comprises one polar solvent.

B206.1. The pharmaceutical composition of embodiment B206, wherein the polar solvent component is NMP.

B207. The pharmaceutical composition of any one of embodiments B196-B205, wherein the polar solvent component comprises two polar solvents each in a percent volume (v/v) amount in the pharmaceutical composition, wherein the sum of the percent volume of each of the polar solvents in the pharmaceutical composition is 100%.

B208. The pharmaceutical composition of embodiment B207, comprising about 1% to about 20% (v/v) PEG300 and about 80% to about 99% (v/v) NMP.

B209. The pharmaceutical composition of embodiment B207, comprising about 1% to about 50% (v/v) PEG400 and about 50% to about 99% (v/v) NMP.

B210. The pharmaceutical composition of embodiment B207, comprising about 90% to about 99% (v/v) NMP and about 1% to about 10% (v/v) DMSO.

B211. The pharmaceutical composition of embodiment B207, comprising about 94% to about 99% (v/v) NMP and about 1% to about 6% (v/v) ethanol.

B212. The pharmaceutical composition of any one of embodiments B196-B205, wherein the polar solvent component comprises three polar solvents each in a percent volume (v/v) amount in the pharmaceutical composition, wherein the sum of the percent volume of each of the polar solvents in the pharmaceutical composition is 100%.

B213. The pharmaceutical composition of embodiment B212, comprising about 10% to about 80% (v/v) NMP, about 1% to about 15% (v/v) DMSO and about 1% to about 15% (v/v) ethanol.

B214. The pharmaceutical composition of embodiment B212, comprising about 10% to about 80% (v/v) NMP, about 1% to about 15% (v/v) ethanol and about 1% to about 20% (v/v) PEG300.

B215. The pharmaceutical composition of embodiment B212, comprising about 10% to about 80% (v/v) NMP, about 1% to about 15% (v/v) ethanol and about 1% to about 50% (v/v) PEG400.

B216. The pharmaceutical composition of any one of embodiments B196-B205, wherein the polar solvent component comprises four polar solvents each in a percent volume (v/v) amount in the pharmaceutical composition, wherein the sum of the percent volume of each of the polar solvents in the pharmaceutical composition is 100%.

B217. The pharmaceutical composition of embodiment B216, comprising about 1% to about 20% (v/v) PEG300, about 10% to about 80% (v/v) NMP, about 1% to about 15% (v/v) DMSO and about 1% to about 15% (v/v) ethanol.

B217.1. The pharmaceutical composition of embodiment B216, comprising about 1% to about 50% (v/v) PEG400, about 10% to about 40% (v/v) NMP, about 1% to about 15% (v/v) DMSO and about 1% to about 15% (v/v) ethanol.

B218. The pharmaceutical composition of any one of embodiments B196-B218, wherein one or more of the polar solvents is at least 90% pure.

B219. The pharmaceutical composition of any one of embodiments B22-B26, comprising no aqueous component.

B220. The pharmaceutical composition of any one of embodiments B22-B26 and B219, comprising no water or no added water.

B221. The pharmaceutical composition of any one of embodiments B22-B26 and any one of embodiments B219 or B220, comprising no surfactant component.

B222. The pharmaceutical composition of any one of embodiments B22-B26 and any one of embodiments B219-B221, comprising no solvent component.

B223. The pharmaceutical composition of any one of embodiments B22-B26 and any one of embodiments B219-B222, comprising no polar solvent component.

B224. The pharmaceutical composition of any one of embodiments B22-B26 and any one of embodiments B219-B223, consisting essentially of or consisting of the oil and the active ingredient.

B225. The pharmaceutical composition of any one of embodiments B22-B26 and any one of embodiments B219-B224, wherein the oil component comprises polyethoxylated castor oil (PECO), castor oil or cottonseed oil.

B226. The pharmaceutical composition of embodiment B225, wherein the PECO is Cremophor EL®.

B227. The pharmaceutical composition of any one of embodiments B22-B26 and any one of embodiments B219-B226, wherein the oil component is at least 90% pure.

B228. The pharmaceutical composition of any one of embodiments B22-B26 and any one of embodiments B219-B227, comprising about 5 milligrams per gram (mg/g) to about 200 mg/g, weight to weight, of a compound having a structure according to Formula A or Formula B.

B229. The pharmaceutical composition of embodiment B228, comprising about 20 mg/g or greater, weight to weight, of a compound having a structure according to Formula A or Formula B.

B230. The pharmaceutical composition of embodiment B228 or B229, comprising about 5 mM to about 140 mM of a compound having the structure of Formula A or Formula B.

B231. The pharmaceutical composition of embodiment B230, comprising about 28 mM or more of a compound having a structure according to Formula A or Formula B.

B232. The pharmaceutical composition of any one of embodiments B22-B26 and any one of embodiments B219-B221, comprising a polar solvent component comprising one or more polar solvents.

B233. The pharmaceutical composition of embodiment B232, comprising about 5 milligrams per gram (mg/g) to about 200 mg/g, weight to weight, of a compound having a structure according to Formula A or Formula B.

B234. The pharmaceutical composition of embodiment B233, comprising about 20 mg/g or greater, weight to weight, of a compound having a structure according to Formula A or Formula B.

B235. The pharmaceutical composition of embodiment B233 or B234, comprising about 5 mM to about 140 mM of a compound having the structure of Formula A or Formula B.

B236. The pharmaceutical composition of embodiment B235, comprising about 28 mM or more of a compound having a structure according to Formula A or Formula B.

B237. The pharmaceutical composition of any one of embodiments B232-B236, wherein the solvent component comprises one or more of N-methyl 2-pyrrolidone (NMP), polyethylene glycol 300 (PEG300), polyethylene glycol 400 (PEG400), dimethyl sulfoxide (DMSO), 2-(2-ethoxyethoxy)ethanol (EEE) or ethanol.

B238. The pharmaceutical composition of embodiment B237, wherein the EEE is Transcutol®.

B239. The pharmaceutical composition of any one of embodiments B232-B238, comprising one polar solvent.

B240. The pharmaceutical composition of embodiment B239, wherein the polar solvent component is NMP.

B241. The pharmaceutical composition of embodiment B239, comprising about 80% to about 99% (v/v) oil component and about 1% to about 20% (v/v) PEG300.

B242. The pharmaceutical composition of embodiment B239, comprising about 51% to about 99% (v/v) oil component and about 1% to about 49% (v/v) PEG400.

B243. The pharmaceutical composition of embodiment B239, comprising about 51% to about 99% (v/v) oil component and about 1% to about 49% (v/v) NMP.

B244. The pharmaceutical composition of embodiment B239, comprising about 90% to about 99% (v/v) oil component and about 1% to about 10% (v/v) DMSO.

B245. The pharmaceutical composition of embodiment B239, comprising about 94% to about 99% (v/v) NMP and about 1% to about 6% (v/v) ethanol.

B246. The pharmaceutical composition of any one of embodiments B232-B238, comprising two polar solvents each in a percent volume (v/v) amount in the pharmaceutical composition, wherein the sum of the percent volume of each of the oil component and polar solvents in the pharmaceutical composition is 100%.

B247. The pharmaceutical composition of embodiment B246, comprising about 50% to about 98% (v/v) oil component, about 1% to about 20% (v/v) PEG300 and about 1% to about 40% (v/v) NMP.

B248. The pharmaceutical composition of embodiment B246, comprising about 50% to about 98% (v/v) oil component, about 1% to about 20% (v/v) PEG400 and about 1% to about 40% (v/v) NMP.

B249. The pharmaceutical composition of embodiment B246, comprising about 50% to about 98% (v/v) oil component, about 1% to about 40% (v/v) NMP and about 1% to about 10% (v/v) DMSO.

B250. The pharmaceutical composition of embodiment B246, comprising about 50% to about 98% (v/v) oil component, about 1% to about 40% (v/v) NMP and about 1% to about 6% (v/v) ethanol.

B251. The pharmaceutical composition of embodiment B246, comprising about 50% to about 98% (v/v) oil component, about 1% to about 20% (v/v) PEG300 and about 1% to about 10% (v/v) DMSO.

B252. The pharmaceutical composition of embodiment B246, comprising about 50% to about 98% (v/v) oil component, about 1% to about 20% (v/v) PEG400 and about 1% to about 10% (v/v) DMSO.

B253. The pharmaceutical composition of embodiment B246, comprising about 50% to about 98% (v/v) oil component, about 1% to about 20% (v/v) PEG300 and about 1% to about 6% (v/v) ethanol.

B254. The pharmaceutical composition of embodiment B246, comprising about 50% to about 98% (v/v) oil component, about 1% to about 20% (v/v) PEG400 and about 1% to about 6% (v/v) ethanol.

B255. The pharmaceutical composition of any one of embodiments B232-B238, comprising three polar solvents each in a percent volume (v/v) amount in the pharmaceutical composition, wherein the sum of the percent volume of each of the oil component and polar solvents in the pharmaceutical composition is 100%.

B256. The pharmaceutical composition of embodiment B255, comprising about 50% to about 97% (v/v) oil component, about 1% to about 40% (v/v) NMP, about 1% to about 10% (v/v) DMSO and about 1% to about 6% (v/v) ethanol.

B257. The pharmaceutical composition of embodiment B255, comprising about 50% to about 97% (v/v) oil component, about 1% to about 40% (v/v) NMP, about 1% to about 6% (v/v) ethanol and about 1% to about 20% (v/v) PEG300.

B258. The pharmaceutical composition of embodiment B255, comprising about 50% to about 97% (v/v) oil component, about 1% to about 40% (v/v) NMP, about 1% to about 6% (v/v) ethanol and about 1% to about 50% (v/v) PEG400.

B259. The pharmaceutical composition of embodiment B255, comprising about 50% to about 97% (v/v) oil component, about 1% to about 20% (v/v) PEG300, about 1% to about 10% (v/v) DMSO and about 1% to about 6% (v/v) ethanol.

B260. The pharmaceutical composition of embodiment B255, comprising about 50% to about 97% (v/v) oil component, about 1% to about 50% (v/v) PEG300, about 1% to about 10% (v/v) DMSO and about 1% to about 6% (v/v) ethanol.

B261. The pharmaceutical composition of any one of embodiments B232-B238, comprising four polar solvents each in a percent volume (v/v) amount in the pharmaceutical composition, wherein the sum of the percent volume of each of the oil component and polar solvents in the pharmaceutical composition is 100%.

B262. The pharmaceutical composition of embodiment B255, comprising about 50% to about 96% (v/v) oil component, about 1% to about 20% (v/v) PEG300, about 1% to about 40% (v/v) NMP, about 1% to about 15% (v/v) DMSO and about 1% to about 6% (v/v) ethanol.

B263. The pharmaceutical composition of embodiment B255, comprising about 50% to about 96% (v/v) oil component, about 1% to about 20% (v/v) PEG400, about 1% to about 40% (v/v) NMP, about 1% to about 15% (v/v) DMSO and about 1% to about 6% (v/v) ethanol.

B264. The pharmaceutical composition of any one of embodiments B232-B264, wherein the oil component comprises polyethoxylated castor oil (PECO), castor oil or cottonseed oil.

B265. The pharmaceutical composition of embodiment B264, wherein the PECO is Cremophor EL®.

B266. The pharmaceutical composition of any one of embodiments B232-B265, wherein the oil component is at least 90% pure.

B267. The pharmaceutical composition of any one of embodiments B232-B266, wherein each of the one or more of the polar solvents is at least 90% pure.

B268. The pharmaceutical composition of any one of embodiments B0.1-B267, in dosage form.

B269. The pharmaceutical composition of any one of embodiments B0.1-B268, in subcutaneous administration dosage form.

B270. The pharmaceutical composition of any one of embodiments B0.1-B269, for subcutaneous administration.

B271. The pharmaceutical composition of any one of embodiments B0.01-B267, in intramuscular administration dosage form.

B272. The pharmaceutical composition of any one of embodiments B0.01-B267, for intramuscular administration.

B273. The pharmaceutical composition of any one of embodiments B268-B272, in an auto-injector pen device.

C1. A pharmaceutical composition, comprising an active ingredient and a solubilizing excipient component, wherein: the active ingredient comprises a compound having the structure of Formula A, a compound of Formula B, or a rimiducid analog; and the solubilizing excipient component comprises one or more of ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

C2. The pharmaceutical composition of embodiment A1, wherein the solubilizing excipient component consists of ethyl acetate, ethanol, isopropyl myristate (IPM), dimethyl isosorbide (DMI), 2-(2-ethoxyethoxy)ethanol (EEE), caprylic/capric triglycerides (CCT) or propylene glycol (PG).

C3. The pharmaceutical composition of embodiment C1, wherein the solubilizing excipient component comprises ethyl acetate.

C4. The pharmaceutical composition of embodiment C1, wherein the solubilizing excipient component comprises ethanol.

C5. The pharmaceutical composition of embodiment C1, wherein the solubilizing excipient component comprises IPM.

C6. The pharmaceutical composition of embodiment C1, wherein the solubilizing excipient component comprises DMI.

C7. The pharmaceutical composition of embodiment C1, wherein the solubilizing excipient component comprises EEE.

C8. The pharmaceutical composition of embodiment C7, wherein the EEE is Transcutol®.

C9. The pharmaceutical composition of embodiment C1, wherein the solubilizing excipient component comprises CCT.

C10. The pharmaceutical composition of embodiment C1, wherein the solubilizing excipient component comprises propylene glycol (PG).

C11. The pharmaceutical composition of any one of embodiments C1 and C2-C10, wherein the solubilizing excipient component comprises two components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

C12. The pharmaceutical composition of embodiment C11, wherein a first component and a second component are at a ratio of about 40:60 (v:v) to about 60:40 (v:v).

C13. The pharmaceutical composition of embodiment C12, wherein the first component and the second component are at a ratio of about 50:50 (v:v).

C14. The pharmaceutical composition of any on one of embodiments C11-C13, wherein the solubilizing excipient component comprises ethyl acetate and ethanol.

C15. The pharmaceutical composition of any on one of embodiments C11-C13, wherein the solubilizing excipient component comprises IPM and ethanol.

C16. The pharmaceutical composition of any one of embodiments C1 and C2-C10, wherein the solubilizing excipient component comprises four components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

C17. The pharmaceutical composition of embodiment C16, wherein the solubilizing excipient component comprises ethyl acetate, ethanol, DMI and EEE.

C18. The pharmaceutical composition of embodiment C17, wherein the solubilizing excipient component comprises ethyl acetate, ethanol, DMI and EEE (20-30:20-30:10-20:30-40, v:v:v:v).

C19. The pharmaceutical composition of embodiment C18, wherein the solubilizing excipient component comprises ethyl acetate, ethanol, DMI and EEE (25:25:15:35, v:v:v:v) C20. The pharmaceutical composition of embodiment C16, wherein the solubilizing excipient component comprises IPM, ethanol, EEE and DMI.

C21. The pharmaceutical composition of embodiment C20, wherein the solubilizing excipient component comprises IPM, ethanol, EEE and DMI (30-40:10-20:30-40:10-20, v:v:v:v:v).

C22. The pharmaceutical composition of embodiment C21, wherein the solubilizing excipient component comprises IPM, ethanol, EEE and DMI (35:15:35:15, v:v:v:v).

C23. The pharmaceutical composition of embodiment C16, wherein the solubilizing excipient component comprises CCT, ethanol, EEEE and DMI.

C24. The pharmaceutical composition of embodiment C23, wherein the solubilizing excipient component comprises CCT, ethanol, EEEE and DMI (30-40:10-20:30-40:10-20, v:v:v:v).

C25. The pharmaceutical composition of embodiment C24, wherein the solubilizing excipient component comprises CCT, ethanol, EEEE and DMI (35:15:35:15, v:v:v:v).

C26. The pharmaceutical composition of embodiment C16, wherein the solubilizing excipient component comprises IPM, PG, EEE, and DMI.

C27. The pharmaceutical composition of embodiment C26, wherein the solubilizing excipient component comprises IPM, PG, EEE, and DMI (35-45:5-15:30-40:10-20, v:v:v:v).

C28. The pharmaceutical composition of embodiment C27, wherein the solubilizing excipient component comprises IPM, PG, EEE, and DMI (40:10:35:15, v:v:v:v).

C29. The pharmaceutical composition of embodiment C16, wherein the solubilizing excipient component comprises ethyl acetate, IPM, ethanol, DMI and EEE.

C30. The pharmaceutical composition of embodiment C29, wherein the solubilizing excipient component comprises ethyl acetate, IPM, ethanol, DMI and EEE (10-20:10-20:20-30:10-20:25-35, v:v:v:v).

C31. The pharmaceutical composition of embodiment C30, wherein the solubilizing excipient component comprises ethyl acetate, IPM, ethanol, DMI and EEE (15:15:25:15:30, v:v:v:v).

C32. The pharmaceutical composition of any one of embodiments C1 and C2-C10, wherein the solubilizing excipient component comprises five components chosen from ethyl acetate, ethanol, isopropyl myristate (IPM), dimethyl isosorbide (DMI), 2-(2-ethoxyethoxy)ethanol (EEE), caprylic/capric triglycerides (CCT) and propylene glycol (PG).

C33. The pharmaceutical composition of embodiment C32, wherein the solubilizing excipient component comprises CCT, ethanol, DMI, EEE and PG.

C34. The pharmaceutical composition of embodiment C33, wherein the solubilizing excipient component comprises CCT, ethanol, DMI, EEE and PG (20-30:20-30:10-20:25-35:1-10, v:v:v:v:v).

C35. The pharmaceutical composition of embodiment C34, wherein the solubilizing excipient component comprises CCT, ethanol, DMI, EEE and PG (25:25:15:30:5, v:v:v:v:v).

C36. The pharmaceutical composition of embodiment C20, comprising isopropyl myristate (20-50 wt %), ethanol (5-25 wt %), EEE (25-50 wt %), dimethyl isosorbide (5-25 wt %) and rimiducid (2-10 wt %).

C37. The pharmaceutical composition of any one of embodiments C1-C36, in dosage form.

C38. The pharmaceutical composition of any one of embodiments C1-C37, in topical dosage form.

C39. The pharmaceutical composition of any one of embodiments C1-C38, for topical administration.

D1. The pharmaceutical composition of any one of embodiments B0.1-B201 and C1-C39, for multimerization of a fusion protein, wherein the fusion protein comprises a polypeptide that binds to the compound having the structure of Formula A, the compound having the structure of Formula B, or the rimiducid analog.

D1.1. A method for administering a pharmaceutical composition of any one of embodiments B0.1-B201 and C1-C39 to cells comprising a fusion protein, wherein the fusion protein comprises a polypeptide that binds to the compound having the structure of Formula A, the compound having the structure of Formula B, or the rimiducid analog.

D1.2. The method of embodiment D1.1, wherein the pharmaceutical composition is administered to a subject in need thereof.

D2. The pharmaceutical composition of embodiment D1 or the method of embodiment D1.1 or D1.2, wherein the polypeptide that binds to the compound having the structure of Formula A, the compound having the structure of Formula B, or the rimiducid analog, is FKBP12-V36.

D3. The pharmaceutical composition or the method of embodiment D2, wherein the FKBP12-V36 comprises the polypeptide of SEQ ID NO:1 or SEQ ID NO:4, or a polypeptide at least 90% identical to the polypeptide of SEQ ID NO:1 or SEQ ID NO:4.

D4. The pharmaceutical composition or the method of any one of embodiments D1-D3, wherein the fusion protein comprises a polypeptide capable of inducing cell death.

D5. The pharmaceutical composition or the method of embodiment D4, wherein the polypeptide capable of inducing cell death is caspase-9.

D6. The pharmaceutical composition or the method of embodiment D5, wherein the caspase-9 is a truncated caspase-9.

D7. The pharmaceutical composition or the method of embodiment D6, wherein the truncated caspase-9 comprises the polypeptide of SEQ ID NO:3, or a polypeptide at least 90% identical to SEQ ID NO:3.

D8. The pharmaceutical composition of the method of any one of embodiments D1-D7, wherein the fusion protein comprises the polypeptides of SEQ ID NO:1, SEQ ID NO:2 and SEQ ID NO:3, or polypeptides at least 90% identical to the polypeptides of SEQ ID NO:1, SEQ ID NO:2 and SEQ ID NO:3.

D9. The pharmaceutical composition or the method of embodiment D8, wherein the fusion protein comprises, in the N-terminus to C-terminus direction, the polypeptides of SEQ ID NO:1, SEQ ID NO:2 and SEQ ID NO:3, or polypeptides at least 90% identical to the polypeptides of SEQ ID NO:1, SEQ ID NO:2 and SEQ ID NO:3.

D10. The pharmaceutical composition or method of any one of embodiments D1-D3, wherein the fusion protein comprises a polypeptide capable of activating immune cells.

D11. The pharmaceutical composition or the method of embodiment D10, wherein the polypeptide capable of activating immune cells comprises a MyD88 polypeptide or truncated MyD88 polypeptide.

D12. The pharmaceutical composition or method of embodiment D11, wherein the truncated MyD88 polypeptide comprises the polypeptide of SEQ ID NO:5, or a polypeptide at least 90% identical to SEQ ID NO:5.

D13. The pharmaceutical composition or the method of any one of embodiments D10-D12, wherein the polypeptide capable of activating immune cells comprises a CD40 polypeptide or cytoplasmic CD40 polypeptide.

D14. The pharmaceutical composition or method of embodiment D13, wherein the cytoplasmic CD40 polypeptide comprises the polypeptide of SEQ ID NO:6, or a polypeptide at least 90% identical to SEQ ID NO:7.

D15. The pharmaceutical composition or the method of any one of embodiments D10-D15, wherein the fusion protein comprises the polypeptides of SEQ ID NO:4, SEQ ID NO:5 and SEQ ID NO:6, or polypeptides at least 90% identical to the polypeptides of SEQ ID NO:4, SEQ ID NO:5 and SEQ ID NO:6.

D16. The pharmaceutical composition or the method of embodiment D15, wherein the fusion protein comprises, in the N-terminus to C-terminus direction, the polypeptides of SEQ ID NO:5, SEQ ID NO:6 and SEQ ID NO:4, or polypeptides at least 90% identical to the polypeptides of SEQ ID NO:5, SEQ ID NO:6 and SEQ ID NO:4.

D17. The pharmaceutical composition or the method of embodiment D16, wherein:
there is a linker peptide between (i) the polypeptide of SEQ ID NO:5, or the polypeptide at least 90% identical to SEQ ID NO:5, and (ii) the polypeptide of SEQ ID NO:6, or the polypeptide at least 90% identical to SEQ ID NO:6; or
there is a linker peptide between (i) the polypeptide of SEQ ID NO:6, or the polypeptide at least 90% identical to SEQ ID NO:6, and (ii) the polypeptide of SEQ ID NO:4, or the polypeptide at least 90% identical to SEQ ID NO:4.

D18. The pharmaceutical composition of the method of any one of embodiments D1-D17, wherein the fusion protein comprises a membrane association polypeptide.

D19. The pharmaceutical composition of the method of embodiment D18, wherein the fusion protein comprises a myristoyl association polypeptide.

D20. The pharmaceutical composition or the method of embodiment D19, wherein the fusion protein comprises a membrane association polypeptide of SEQ ID NO:7, or a polypeptide at least 90% identical to the polypeptide of SEQ ID NO:7.

D21. The pharmaceutical composition or the method of any one of embodiments D18-D20, wherein the membrane association polypeptide is positioned at the N-terminus of the fusion protein.

D22. The pharmaceutical composition or the method of embodiment D21, wherein the fusion protein optionally contains a linker peptide at the C-terminus of the membrane association polypeptide.

D23. The pharmaceutical composition of the method of any one of embodiments D1-D22, wherein the fusion protein is expressed in cells.

D24. The pharmaceutical composition or the method of embodiment D23, wherein the cells are in vitro or ex vivo.

D25. The pharmaceutical composition of the method of embodiment D23, wherein the cells are in vivo.

D25.1. The pharmaceutical composition or the method of any one of embodiments D1-D25, for eliminating cells containing a fusion protein, wherein the fusion protein comprises a polypeptide that binds to the compound having the structure of Formula A or Formula B or a rimiducid analog.

D26. The pharmaceutical composition or the method of any one of embodiments D1-D25.1, for treatment of a cell proliferative condition.

D27. The pharmaceutical composition or the method of embodiment D26, wherein the condition is a cancer.

D28. The pharmaceutical composition or the method of any one of embodiments D1-D27, for treatment of a skin condition.

D29. The pharmaceutical composition or the method of embodiment D28, wherein the skin condition is chosen from skin lesion, actinic keratosis, skin carcinoma and Bowen's disease.

D30. The pharmaceutical composition or the method of any one of embodiments D1-D28, for treatment of a pathogen condition.

D31. The pharmaceutical composition of the method of embodiment D30, wherein the pathogen condition is a viral condition or bacterial condition.

D32. The pharmaceutical composition of any one of embodiments B0.1-B201 and C1-C39, in unit dosage form.

E1. A device, comprising the pharmaceutical composition of any one of embodiments B0.1-B201 and C1-C39.

E2. The device of embodiment E1, which is an injectable device or which is not an injectable device.

E3. The device of embodiment E1, which is an auto-injection device.

E4. The device of embodiment E3, which is a pen auto-injection device.

E5. The device of embodiment E1, wherein the device is a topically administered device.

E6. The device of embodiment E5, wherein the device is a dressing.

E7. The device of embodiment E5, wherein the device is a patch.

F1. A method for manufacturing a pharmaceutical composition, comprising: combining an active ingredient with an oil component, wherein the active ingredient comprises a compound having the structure of Formula A, a compound having the structure of Formula B, or a rimiducid analog.

F1.1. The method of embodiment F1, wherein a mixture of the active ingredient and the oil component is prepared and the mixture is exposed to:
conditions in which the temperature is above room temperature, or
agitation conditions, or
agitation conditions and conditions in which the temperature is above room temperature.

F1.2. The method of embodiment F1.1, wherein the temperature is about 40 degrees Celsius to about 60 degrees Celsius.

F1.3. The method of embodiment F1.2, wherein the temperature is about 50 degrees Celsius.

F1.4. The method of any one of embodiments F1.1-F1.3, wherein the mixture is exposed to conditions in which the temperature is above room temperature for about 1 hour to about 5 hours.

F1.5. The method of any one of embodiments F1.1-F1.4, wherein the mixture is exposed to conditions in which the temperature is above room temperature for about 3 hours.

F1.6. The method of any one of embodiments F1.1-F1.5, wherein the mixture is exposed to agitation conditions for about 12 hours to about 36 hours.

F1.7. The method of any one of embodiments F1.1-F1.6, wherein the mixture is exposed to agitation conditions for about 24 hours.

F1.8. The method of any one of embodiments F1-F1.7, comprising manufacturing the pharmaceutical composition of any one of embodiments B219-B231.

F2. A method for manufacturing a pharmaceutical composition, comprising: combining an active ingredient with an oil component and with a surfactant component or a solvent component, wherein the active ingredient comprises a compound having the structure of Formula A, a compound having the structure of Formula B, or a rimiducid analog.

F3. The method of embodiment F2, comprising:
(a) combining the active ingredient with the oil component, thereby generating an oil mixture;
(b) combining the surfactant component or the solvent component with the aqueous component, thereby generating an aqueous mixture; and
(c) after (a) and (b), combining the oil mixture with the aqueous mixture.

F4. The method of any one of embodiments F1-F3, wherein no aqueous component is added.

F5. The method of any one of embodiments F1-F3, wherein an aqueous component is added and the amount of the aqueous component in the pharmaceutical composition is 10% by volume or less.

F6. The method of any one of embodiments F1-F5, comprising manufacturing a pharmaceutical composition of any one of embodiments B0.1-B21.9.

F7. The method of any one of embodiments F1-F5, comprising manufacturing a pharmaceutical composition of any one of embodiments B22-B43.

F8. A method for manufacturing a pharmaceutical composition, comprising: combining an active ingredient with an aqueous component, a surfactant component and an oil component, wherein the active ingredient comprises a compound having the structure of Formula A, a compound having the structure of Formula B, or a rimiducid analog.

F9. The method of embodiment F8, comprising:
(a) combining the active ingredient with the oil component, thereby generating an oil mixture;
(b) combining the surfactant component with the aqueous component, thereby generating an aqueous mixture; and
(c) after (a) and (b), combining the oil mixture with the aqueous mixture.

F10. The method of embodiment F8 or F9, comprising manufacturing a pharmaceutical composition of any one of embodiments B104-B119.

F11. The method of embodiment F8 or F9, comprising manufacturing a pharmaceutical composition of any one of embodiments B120-B139.

F12. A method for manufacturing a pharmaceutical composition, comprising: combining an active ingredient with an aqueous component, a solvent component, a surfactant component and an oil component, wherein the active ingredient comprises a compound having the structure of Formula A, a compound having the structure of Formula B, or a rimiducid analog.

F13. The method of embodiment F12, comprising:
(a) combining the active ingredient with the oil component, thereby generating an oil mixture;
(b) combining the surfactant component, the solvent component and the aqueous component, thereby generating an aqueous mixture; and
(c) after (a) and (b), combining the oil mixture with the aqueous mixture.

F14. The method of embodiment F12 or F13, comprising manufacturing a pharmaceutical composition of any one of embodiments B140-B162.

F15. The method of embodiment F12 or F13, comprising manufacturing a pharmaceutical composition of any one of embodiments B163-B180.

F16. The method of any one of embodiments F3-F15, wherein (i) the oil mixture is, or (ii) the aqueous mixture is, or (iii) the oil mixture and the aqueous mixture are, exposed to conditions in which the temperature is above room temperature.

F17. The method of embodiment F16, wherein the temperature is about 40 degrees Celsius to about 80 degrees Celsius.

F18. The method of embodiment F16, wherein the temperature is about 50 degrees Celsius to about 70 degrees Celsius.

F19. The method of any one of embodiments F5-F18, wherein the pharmaceutical composition is an oil-in-water emulsion comprising particles.

F20. The method of any one of embodiments F5-F19, wherein:
an emulsion containing particles is formed, and
the method does not include exposing the particles to conditions that reduce the size of the particles.

F21. The method of any one of embodiments F5-F19, wherein:
an emulsion containing particles is formed, and
the method comprises exposing the particles to conditions that reduce the size of the particles.

F22. The method of embodiment F20 or F21, wherein the conditions that reduce the size of the particles comprise sonication conditions, micro-fluidization conditions, or ball-milling conditions.

F23. The method of any one of embodiments F19-F22, wherein the particles are defined by a mean particle diameter of about 50 nanometers (nm) to about 2000 nm.

F24. The method of embodiment F23, wherein the particles have one or more of the following characteristics: (i) a mean particle diameter of less than or equal to 100 nm; (ii) a standard deviation of the mean particle diameter of about 20 nm or less; (iii) a dispersity of about 0.10 to about 0.25; and (iv) a standard deviation of the dispersity of about 0.10 or less.

F25. A method for manufacturing a pharmaceutical composition, comprising: combining an active ingredient with an aqueous component, a solvent component and a surfactant component, wherein the active ingredient comprises a compound having the structure of Formula A, a compound having the structure of Formula B, or a rimiducid analog.

F26. The method of embodiment F25, comprising manufacturing a pharmaceutical composition of any one of embodiments B59 and B181-B195.

F27. The method of any one of embodiments F2-F26, comprising manufacturing a pharmaceutical composition of any one of embodiments B60-B103.

F28. A method for manufacturing a pharmaceutical composition, comprising: combining an active ingredient with one or more polar solvents, wherein the active ingredient comprises a compound having the structure of Formula A, a compound having the structure of Formula B, or a rimiducid analog.

F29. The method of embodiment F28, comprising:
(a) combining two or more polar solvents, thereby generating a first mixture; and
(b) combining the active ingredient with the polar solvent mixture, thereby generating a second mixture.

F30. The method of embodiment F29, wherein the first mixture, or the second mixture, or the first mixture and the second mixture are exposed to:
conditions in which the temperature is above room temperature, or
agitation conditions, or
agitation conditions and conditions in which the temperature is above room temperature.

F31. The method of embodiment F32, wherein the temperature is about 40 degrees Celsius to about 60 degrees Celsius.

F32. The method of embodiment F31, wherein the temperature is about 50 degrees Celsius.

F33. The method of any one of embodiments F30-F32, wherein the first mixture, or the second mixture, or the first mixture and the second mixture, is exposed to conditions in which the temperature is above room temperature for about 1 hour to about 5 hours.

F34. The method of any one of embodiments F33, wherein the first mixture, or the second mixture, or the first mixture and the second mixture, is exposed to conditions in which the temperature is above room temperature for about 3 hours.

F35. The method of any one of embodiments F30-F34, wherein the first mixture, or the second mixture, or the first mixture and the second mixture, is exposed to agitation conditions for about 12 hours to about 36 hours.

F36. The method of any one of embodiments F30-F35, wherein the first mixture, or the second mixture, or the first mixture and the second mixture, is exposed to agitation conditions for about 24 hours.

F37. The method of any one of embodiments F28-F36, comprising manufacturing the pharmaceutical composition of any one of embodiments B196-B218.

F38. A method for manufacturing a pharmaceutical composition, comprising: combining an active ingredient with an oil component and one or more polar solvents, wherein the active ingredient comprises a compound having the structure of Formula A, a compound having the structure of Formula B, or a rimiducid analog.

F39. The method of embodiment F38, comprising:
(a) combining the active ingredient with the polar solvent mixture, thereby generating a first mixture; and
(b) combining the first mixture with the oil component, thereby generating a second mixture.

F40. The method of embodiment F39, wherein the first mixture, or the second mixture, or the first mixture and the second mixture, is exposed to:
conditions in which the temperature is above room temperature, or
agitation conditions, or
agitation conditions and conditions in which the temperature is above room temperature.

F41. The method of embodiment F40, wherein the temperature is about 40 degrees Celsius to about 60 degrees Celsius.

F42. The method of embodiment F41, wherein the temperature is about 50 degrees Celsius.

F43. The method of any one of embodiments F39, wherein the first mixture, or the second mixture, or the first mixture and the second mixture, is exposed to conditions in which the temperature is above room temperature for about 1 hour to about 5 hours.

F44. The method of any one of embodiments F43, wherein the first mixture, or the second mixture, or the first mixture and the second mixture, is exposed to conditions in which the temperature is above room temperature for about 3 hours.

F45. The method of any one of embodiments F40-F44, wherein the first mixture, or the second mixture, or the first mixture and the second mixture, is exposed to agitation conditions for about 12 hours to about 36 hours.

F46. The method of any one of embodiments F40-F45, wherein the first mixture, or the second mixture, or the first mixture and the second mixture, is exposed to agitation conditions for about 24 hours.

F47. The method of any one of embodiments F39-F46, comprising manufacturing the pharmaceutical composition of any one of embodiments B232-B267.

F48. A method for manufacturing a pharmaceutical composition, comprising: combining an active ingredient with a solubilizing excipient component, wherein the active ingredient comprises a compound having the structure of Formula A, a compound having the structure of Formula B, or a rimiducid analog.

F49. The method of embodiment F48, comprising manufacturing a pharmaceutical composition of any one of embodiments C1-C39.

F50. The method of any one of embodiments F1-F49, comprising manufacturing the pharmaceutical composition in a dosage form.

F51. The method of embodiment F50, wherein the dosage form is a subcutaneous administration dosage form.

F52. The method of embodiment F51, wherein the pharmaceutical composition is for subcutaneous administration.

F53. The method of embodiment F50, wherein the dosage from is an intramuscular administration dosage form.

F54. The method of embodiment F53, wherein the pharmaceutical composition is for intramuscular administration.

F55. The method of any one of embodiments F50-F54, comprising loading the pharmaceutical composition into an auto-injector pen device.

F56. The method of embodiment F50, wherein the dosage form is a topical administration dosage form.

F57. The method of embodiment F56, wherein the pharmaceutical composition is for topical administration.

F58. A pharmaceutical composition obtainable by a method of any one of embodiments F1-F57.

### Examples

The examples set forth below illustrate certain implementations and do not limit the technology.

### Example 1: Rimiducid Formulations for Subcutaneous (SC) Administration

Described are formulations for pharmaceutical compositions of rimiducid for subcutaneous (SC) administration. The rimiducid utilized as the active ingredient is isomerically pure and has a structure according to Formula B herein. Self-administration of rimiducid via a SC route of administration is facilitated by incorporating the rimiducid SC formulations into an injection-pen, enabling patients to safely and easily self-administer rimiducid subcutaneously, circumventing issues associated with intravenous infusions.

While micelles are formed spontaneously upon interaction of an oral formulation with gastrointestinal fluids, SC formulations are oil-in-water nanoemulsions, formed either spontaneously or by high energy emulsification before injection into patients. High-energy emulsification methods include sonication, lab-scale homogenization (less than one liter (L)), large-scale homogenization (greater than one liter (L)) and the like. Nanoemulsion formulations are formed by first solubilizing rimiducid into an oil-phase excipient. An oil-phase excipient can include one component or a combination of components as shown in Table 1 of this Example.

### Oil-in-Water Emulsions for Pharmaceutical Formulations

Oil-in-water emulsions containing rimiducid (having a structure according to Formula B) are prepared with the following components.

TABLE 1

| Oil Phase | Aqueous Phase | Solubility Enhancers | Penetration Enhancers |
|---|---|---|---|
| Medium chain | Water | lecithin | Transcutol ® |
| triglycerides (MCT) | | | |
| Castor oil | Saline | Polysorbate (PS) | Transcutol ® HP |
| | | 20/40/60/80 | |
| Labrafac ® | | Span 20/40/60/80 | Ethanol |
| Labrafil ® | | Pluronic ® polymers (PP) | |
| Cremophor EL ® | | Propylene glycol (PG) | |
| Triacetin | | Cyclodextrins (CD) | |
| Oleic Acid | | | |
| Capryol ® 90 | | | |
| Medium chain | | | |
| triglycerides:Castor Oil | | | |
| (50:50 w:w) | | | |
| Triacetin:Cremophor | | | |
| EL ® (33:67 w:w) | | | |
| Peceol ®, Span40 | | | |
| Labrasol ®, | | | |
| Lauroglycol ™ 90 | | | |
| Maisine ® CC | | | |
| Pluronic ® polymers | | | |
| (PP) | | | |

Rimiducid is subjected to solubility testing in the oil-phase excipient (e.g., greater than 20 milligrams of rimiducid per 1 gram of excipient; greater than 50 milligrams of rimiducid per 1 gram of excipient; greater than 100 milligrams of rimiducid per 1 gram of excipient). After solubility testing in the oil-phase excipient, rimiducid in an oil-phase excipient is mixed with other excipients including a solubility enhancer, penetration enhancer and an aqueous phase solvent, whereby a coarse emulsion having a particle size greater than 500 nanometers (nm) is formed. Excipients are FDA-approved for pharmaceutical use via the SC route of administration (ROA) or are listed on FDA's GRAS chemicals list.

### Rimiducid Coarse Emulsions

Table 2 of this Example lists excipients and ratios of the coarse emulsions.

TABLE 2

| Excipients | Excipient Ratios (w:w) |
|---|---|
| MCT:Water:PS80:Span80:Ethanol | 30:50:7.5:7.5:5 |
| Castor Oil:Saline:PS60:Transcutol ® | 20:65:10:5 |
| Castor Oil:Saline:PS80 | 15:70:15 |
| Oleic Acid:Saline:Span40:Transcutol ® HP | 35:50:10:5 |
| Cremophor EL ®:Saline:Span80 | 15:75:10 |
| Labrafac ®:Saline:lecithin | 20:50:30 |
| Labrafil ®:Saline:CD:Transcutol ® | 15:65:15:5 |
| Capryol ® 90:Saline:PP:Ethanol | 30:50:15:5 |

Following formation of a coarse emulsion, sonication and lab-scale homogenization is employed to form nanoemulsions (nanoemulsions defined as emulsions with particle size less than or equal to 100 nm in diameter) for lab testing, and large-scale homogenization is employed to form nanoemulsions for large-scale production of nanoemulsions for use in patients and/or large-scale animal studies.

Following solubility testing and emulsion formation, rimiducid formulated in various excipients are dosed subcutaneously to rodents (mice and/or rats) and the pharmacokinetics (PK) of rimiducid are tested. Pharmacokinetic parameters are assessed, such as Cmax values (e.g., plasma levels that can be in the range of 250 to 2500 nanograms (ng) per milliliter (mL)), clearance from circulation (e.g., which can be within or can exceed 120-hours), and PK curves generated for different rimiducid doses (e.g., plasma level (nanograms/milliliter) over time (hours)). Nanoemulsions with particle sizes of less than 100 nm in diameter are expected to result in optimal PK parameters.

A formulation containing the following amounts of rimiducid and additives is tested for a subcutaneous route of administration: castor oil (5-30 wt %), saline (50-75 wt %), polysorbate 80 (15-30 wt %) and rimiducid (2.5-10 wt %).

### Example 2: Single Subcutaneous Dose Pharmacokinetic Study of Rimiducid Formulated in Solution-Based and Emulsion-Based Formulations

Rimiducid was dosed subcutaneously to male CD-IGS rats in one solution-based (i.e., not forming particles) and three emulsion-based (i.e., an oil-in-water emulsion with a measurable particle size) formulations using oil, surfactant, and solvent excipients. The formulations tested were exploratory solution and emulsion formulations. Solutions were formulated by mixing all components and stirring. Emulsions were prepared by mixing to form a coarse emulsion. To form a fine emulsion high-energy sonication was used to reduce particle size to potentially facilitate increased bioavailability and long-term stability. The rimiducid utilized as the active ingredient was isomerically pure and had a structure according to Formula B herein.

Rats were bled using the retro-orbital bleeding (ROB) technique at 1, 2, 4, 8, 24, and 48 hours post-dosing. Following ROB bleeds, plasma was isolated from whole blood, and rimiducid was extracted from plasma and quantified by LCMS using a standard curve prepared with untreated rat plasma spiked with known concentrations of rimiducid.

Rimiducid was dosed to rats at 25 mg/kg using a 2.5-mL/kg dose volume. All formulations were prepared with a final qualitative rimiducid concentration of 10 mg/mL. Any observed precipitation of rimiducid is noted herein.

TABLE 1
Formulation Details.

| Use Case | Formulation | Excipients | Volume Ratio |
|---|---|---|---|
| Solution | sn-A | P400/H₂O/PS80 | 30/40/30 |
| Emulsion | em-A | MCT/P400/H₂O/PS80 | 30/15/40/15 |
| Emulsion | em-B | CO/H₂O/PS80 | 30/60/10 |
| Emulsion | em-C | CO/P400/H₂O/PS80 | 30/15/40/15 |
| Emulsion | em-D | MCT/H₂O/PS80 | 30/60/10 |

In Table 1 of this Example, P400 is Polyethylene Glycol 400, PS80 is Polysorbate 80, MCT is Medium Chain Triglycerides, and CO is Castor Oil.

TABLE 2
Materials and Equipment

| | Description | Manuf. | Cat # |
|---|---|---|---|
| Reagents | | | |
| Rimiducid | Homodimerizer drug | MedChemExpress | Lot: 124865 |
| AP23510 | LCMS internal standard (IS) | MedChemExpress | HY-47001 |
| Methanol | Hypersolv, LCMS grade | VWR | BDH85800.400 |
| Water | Hypersolv, LCMS grade | VWR | BDH83645.400 |
| Formic Acid | HPLC grade | VWR | 97064-706 |
| Polyethylene | Solvent excipient | VWR | AAB21992-30 |
| Glycol 400 | | | |
| Polysorbate 80 | Surfactant excipient | VWR | BDH7781-2 |
| Medium Chain | Oil excipient, derived | Kroger | N/A |
| Triglycerides | from coconut oil | | |
| Castor Oil | Oil excipient, derived from | Kroger | N/A |
| | seed of the castor plant | | |
| Consumables | | | |
| Microcentrifuge | 1.5-mL, snap cap | VWR | 76332-068 |
| tubes, 1.5-mL | | | |
| Needle, disposable | 18 G, 1.5 in | BD | 305918 |
| Syringe, 5 mL | Slip tip | BD (VWR) | BD309647 |
| 22.18-mL Vials | Borosilicate glass, screw cap | VWR | 66011-143 |
| HPLC vials | Convenience kit with vial and screw | VWR | 89523-478 |
| | cap | | |
| HPLC vial inserts | 0.2 mL capacity | MicroSolv (VWR) | 97051-398 |
| Pipette tips - 1000 | Rainin BioClean Ultra, pre-racked, | Rainin | 30389212 |
| μL | with filter | | |
| Pipette tips - 200 μL | Rainin BioClean Ultra, pre-racked, | Rainin | 30389239 |
| | with filter | | |
| Pipette tips - 20 μL | Rainin BioClean Ultra, pre-racked, | Rainin | 17005860 |
| | with filter | | |

| Equipment | Manuf. | Model # | Serial # |
|---|---|---|---|
| Pipette - 2 μL | Rainin, Pipette-Lite LTS | 17014393 | |
| Pipette - 20 μL | Rainin, Pipette-Lite LTS | 17014392 | |
| Pipette - 200 μL | Rainin, Pipette-Lite LTS | 17014391 | |
| Pipette - 1000 μL | Rainin, Pipette-Lite LTS | 17014382 | |
| LCMS, triple quad | AB Sciex | 3200 | 45-1-1610- |
| | | | 2589-2 |
| Stir Bars | ½ inch magnetic stir bar | VWR | 58948-353 |
| LCMS Column | ThermoScientific Aquasil C18, 2.1 × | 77505-052130 | |
| | 50 mm, 5-μm pore size | | |
| High Energy | Qsonica | Q500 | |
| Sonicator | | | |
| Sonicator Microtip | Qsonica | 4418 | |
| Probe, ⅛″ tip | | | |

TABLE 3
Abbreviations

| Abbreviation | Definition |
|---|---|
| HPLC | High-performance liquid chromatography |
| IS | Internal Standard |
| LCMS | Liquid chromatography-mass spectrometry |
| Rim | rimiducid |
| RT | Room temperature |
| ROA | Route of administration |
| ROB | Retro-orbital bleed |
| RSD | Relative Standard Deviation |
| RSE | Relative Standard Error |
| SC | Subcutaneous |

TABLE 4
Study Design

| | |
|---|---|
| Product: | Rimiducid Formulation Development |
| Type of study: | Non-GLP Pharmacokinetic |
| Title of study: | Single Subcutaneous Dose Pharmacokinetic Study (non-GLP) of |
| | Rimiducid Formulated in Solution- and Emulsion-based Formulations |
| Objectives: | There was one main objective in this experiment: |
| | To test the subcutaneous bioavailability of rimiducid formulated in |
| | solution- and emulsion-based formulations in CD-IGS rats: |
| | sn-A - P400/H₂O/PS80, 30/40/30 v/v/v |
| | em-A - MCT/P400/H₂O/PS80, 30/15/40/15 v/v/v/v |
| | em-B - CO/H₂O/PS80, 30/60/10 v/v/v |
| | em-B - CO/P400/H₂O/PS80, 30/15/40/15 v/v/v |
| Animal | Charles River |
| Vendor/CRO: | |
| Species and | CD-IGS Rats |
| strain: | |
| Species sex: | Male |
| Species age or | 300 grams |
| weight at | |
| dosing: | |
| Test article(s): | Rimiducid - P400/H2O/PS80, 30/40/30 v/v/v (sn-A) |
| | Rimiducid - MCT/P400/H₂O/PS80, 30/15/40/15 v/v/v/v (em-A) |
| | Rimiducid - CO/H₂O/PS80, 30/60/10 v/v/v (em-B) |
| | Rimiducid - CO/P400/H₂O/PS80, 30/15/40/15 v/v/v (em-C) |
| Particle size | sn-A - No particle size (solution) |
| details: | em-A - Z-avg - 1229 nm, Pdl - 1 |
| | em-B - Z-avg - 669 nm, Pdl -0.44 |
| | em-C - Z-avg - 185 nm, Pdl - 0.02 |
| Study design: | Rimiducid was subcutaneously (SC) administered to CD-IGS rats (n = 3 |
| | per group) based on Table 5 of this Example. Each animal was |
| | weighed, and the volume of formulation calculated for each rat based |
| | on a mL/kg dose volume. |
| | Rats were monitored for any signs of toxicity or abnormal behavior. |
| | Time points of blood collection post-dosing were as follows (h): 1, 2, 4, |
| | 8, 24, 48 |
| Dose, dose | Rats were dosed SC at a 25 mg/kg rimiducid level. As this is an |
| justification and | exploratory PK experiment, a high dose was used to ensure that |
| route of | plasma values were observed after bioanalysis. |
| administration | |
| (ROA): | |
| Outcome | Endpoints: |
| measures and | Rats were bled using the retro orbital bleeding technique (ROB) at |
| endpoints: | specified time points based on Table 5 of this Example. |
| | Plasma concentrations of rimiducid were measured using pre- |
| | determined LCMS methods compared to known standards. |
| | Rats were monitored for any signs of toxicity or abnormal behavior |
| | after dosing of rimiducid in the new formulations. |
| Study duration: | 3 Days |

### Animal Pre-Conditioning

Rats were weighed the day before dosing (Day −1) to calculate the dose for each rat based on a mL/kg dose volume.

### Route of Drug Administration (ROA)

Rats received a single dose of the drug formulation via subcutaneous (SC) administration.

### Blood Collection

ROB was performed on rats in each group at pre-determined time intervals (see Table 5 of this Example). Following ROB at the terminal time point, rats were euthanized. Blood samples were collected into EDTA-coated (purple top) tubes at specified time points post-drug administration (see Table 5 of this Example).

### Clinical Observations

Rats were observed for adverse clinical signs after dosing of the formulations.
General—nutritional state, quality of hair and coat, behavioral features, and locomotor abnormalities
Head—hair coverage, any abnormal respiratory sounds, discoloration or abnormal discharge from the mouth, position/size of eyeball, discoloration or fluid discharge from eyeball, abnormalities in ear positions or discharge from ear
Neck—mobility, shape, any wounds/masses, guttural sounds
Trunk—position, symmetry, and size of back, thorax, abdomen, or pelvis. Any abnormal movements or respiratory sounds/movements will be noted
Limbs/tail—position, motility, abnormal muscle, proper use (versus lameness).

TABLE 5
Experimental Groups

| | | Dose | API | | | | |
|---|---|---|---|---|---|---|---|
| | | Volume | Dose | | | | Number |
| Group | Formulation | (mL/kg) | (mg/kg) | ROA | Fasting | Time Points (h) | of Rats |
| 1 | sn-A | 2.5 | 25 | SC | N | 1, 2, 4, 8, 24, 48 | 3 |
| 2 | em-A | 2.5 | 25 | SC | N | 1, 2, 4, 8, 24, 48 | 3 |
| 3 | em-B | 2.5 | 25 | SC | N | 1, 2, 4, 8, 24, 48 | 3 |
| 4 | em-C | 2.5 | 25 | SC | N | 1, 2, 4, 8, 24, 48 | 3 |

TABLE 6
Activities

| Date | Activity |
|---|---|
| Day 0 | Body weights for dose volume calculations |
| Day 1 | Drug administration |
| Day 1-Day 2 | Blood collection |
| Day 1-Day 3 | Plasma extraction from whole blood |
| Day 3 | Plasma delivered for bioanalysis |
| Day 3 | Plasma samples received |
| Day 8 | LCMS bioanalysis |

### Dose Formulation Preparation (Emulsions)

Rimiducid was weighed into 20-mL glass vials and weights were recorded. The final target concentrations for all rimiducid SC formulations were 10 mg rimiducid per mL of formulation. Hydrophobic components (i.e., oils (CO, MCT)) were combined with rimiducid and stirred while heating at 60° C. until the rimiducid was dissolved. Stirring/heating took place for a maximum of 30 minutes.

The next steps were performed even if rimiducid had not dissolved (i.e., precipitates were observed). PS80 was dissolved in water and heated to 60° C. while stirring to form an aqueous mixture. When P400 solvent was included, it was mixed with PS80 in water to form an aqueous mixture. After the PS80, and P400 when included, completely dissolved in the water, the aqueous mixture was slowly added to the oil/rimiducid mixture while stirring under heating at 60° C. The vials were heated to 60° C. and stirred with a magnetic stir bar for 2 hours. The coarse emulsions (i.e., not sonicated) were left on the bench top overnight to cool to room temperature.

The following day, the coarse emulsions were observed for phase separation and rimiducid precipitation. Those coarse emulsions that were phase separated were sonicated (see details below). Coarse emulsions in which rimiducid precipitation was observed were excluded from further development.

Coarse emulsions were subjected to sonication after stir bar removal for 5 minutes at room temperature using a 4/2 second on/off pulse pattern and 25% amplitude. A 1/8″ microtip probe was used as the formulation volume was small and sonication was carried out in the same 20-mL glass vials that the coarse emulsions were mixed in.

Particle-size analysis was then carried out on the emulsion formulations by diluting the emulsions 1:100 in pure water and measuring the formulations using a Zetasizer instrument. Refer to Tables 7 and 8 in this Example for formulation and particle size details.

TABLE 7
SC Formulation Excipient Details

| | | Ratio | Rimiducid | | | Stable |
|---|---|---|---|---|---|---|
| | | (vol | Conc. | Sonication | Precipitation | Emulsion |
| Formulation | Components | %) | (mg/mL) | (Y/N) | (Y/N) | (Y/N) |
| sn-A | P400/H₂O/PS80 | 30/40/ | 10 | N | N | N/A, solution |
| | | 30 | | | | |
| em-A | MCT/P400/H₂O/ | 30/15/ | 10 | N | N | Y, no |
| | PS80 | 40/15 | | | | sonication |
| | | | | | | required |
| em-B | CO/H₂O/PS80 | 30/60/ | 10 | Y | N | Y, only after |
| | | 10 | | | | sonication |
| em-C | CO/P400/H₂O/ | 30/15/ | 10 | Y | N | Y, only after |
| | PS80 | 40/15 | | | | sonication |
| em-D | MCT/H₂O/PS80 | 30/60/ | 10 | Y | Initially, all API | Y, only after |
| | | 10 | | | dissolved after | sonication |
| | | | | | stirring at 80° C. | |
| N/A | SO/H₂O/PS80 | 30/60/ | 10 | N | Y, High | N |
| | | 10 | | | amount | |
| N/A | CA/H₂O/PS80 | 30/60/ | 10 | N | Y, High | N |
| | | 10 | | | amount | |
| N/A | CA/P400/H₂O/ | 27/14/ | 10 | Y | N | Y, only after |
| | PS80/SP80 | 36/14/ | | | | sonication |
| | | 9 | | | | |

CO = Castor Oil
SO = Sesame Oil
MCT = Medium Chain Triglycerides
CA = Canola Oil
PS80 = Polysorbate 80
SP80 = Span 80
P400 = PEG400
H2O = Purified Water

Only formulations sn-A. em-A, em-B and em-C were dosed to animals in this study. Formulations described in the bottom three rows of Table 7 of this Example were not considered viable formulations due to high amounts of precipitation, unstable emulsions, or high viscosities.

TABLE 8
Emulsion Particle Size Details

| | | | Particle | | | | | |
|---|---|---|---|---|---|---|---|---|
| | | | Diameter | Average | | | | |
| | | Ratio | (Z-avg, | Z-avg | StDev | | Pdl | Pdl |
| Formulation | Components | (vol %) | nm) | (nm) | (nm) | Pdl | Avg | StDev |
| sn-A | P400/H₂O/PS80 | 30/40/30 | N/A, | N/A | N/A | N/A | N/A | N/A |
| | | | Solution | | | | | |
| em-A | MCT/P400/H₂O/ | 30/15/ | 1234 | 1228.67 | 10.12 | 1 | 1.00 | 0.00 |
| | PS80 | 40/15 | 1217 | | | 1 | | |
| | | | 1235 | | | 1 | | |
| em-B | CO/H₂O/PS80 | 30/60/10 | 643.3 | 668.37 | 22.47 | 0.9 | 0.44 | 0.42 |
| | | | 675.1 | | | 0.336 | | |
| | | | 686.7 | | | 0.084 | | |
| em-C | CO/P400/H₂O/ | 30/15/ | 187.9 | 185.03 | 2.85 | 0.033 | 0.02 | 0.02 |
| | PS80 | 40/15 | 185 | | | 0.007 | | |
| | | | 182.2 | | | 0.007 | | |
| em-D | MCT/H₂O/PS80 | 30/60/10 | 150.3 | 148.60 | 1.96 | 0.136 | 0.12 | 0.02 |
| | | | 149 | | | 0.134 | | |
| | | | 146.5 | | | 0.099 | | |

Pdl—Polydispersity

### LCMS Analysis of In Viva Plasma Samples

LCMS analysis was performed on plasma samples from rats. Plasma concentrations of rimiducid were quantified using the LC/MS method described in Table 9 of this Example (binary gradient of water with 0.1% formic acid and methanol with 0.1% formic acid).

TABLE 9
LCMS Parameters

| Time | % | % | | |
|---|---|---|---|---|
| (minutes) | Water | Methanol | Mass Peaks | |
| 0.00 | 30 | 70 | Rimiducid | Q1: 1419.93 |
| 0.00 | 30 | 70 | | Q3: 151.10 |
| 2.00 | 0 | 100 | AP23510 (IS) | Q1: 1433.95 |
| 2.01 | 0 | 100 | | Q3: 151.00 |
| 3.00 | 30 | 70 | | |
| 5.00 | 30 | 70 | | |

A standard curve was prepared by diluting rimiducid in DMSO and spiking into naïve rat plasma, following Table 10 of this Example. The standards were analyzed, and a linear standard curve was calculated by the LCMS software using area under the curve as the parameter and 1/x² weighting. The ratio of AP1903 Peak Area to IS Peak Area (x-axis) was correlated to AP1903 concentration (y-axis). For sample analysis, 100 μL of plasma isolated from whole blood was added to 100 μL of acetonitrile containing 2.0 μg/mL AP23510 (used as the internal standard). The samples were vortexed and then centrifuged for 10 minutes at 12,000×g to extract rimiducid from rat plasma. Rimiducid concentrations in plasma samples were calculated using the standard curve.

TABLE 10
LCMS Standards

| | | | | | Matrix Standards | | |
|---|---|---|---|---|---|---|---|
| | | | | | | Blank | |
| | 20 × Standard Stocks | | | | | Matrix | |
| | | | | DMSO | | (plasma | |
| | Stock | | Stock | Stock | Volume | from | |
| Std | Volume | DMSO | used | Conc | Stock | untreated | Conc. |
| # | (μL) | (μL) | (μL) | (ng/mL) | (μL) | rats) | (ng/mL) |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| 3 | 100 | 100 | 1000 | 500 | 2 | 38 | 25 |
| 4 | 100 | 100 | 2000 | 1000 | 2 | 38 | 50 |
| [symbol] | [symbol] | [symbol] | [symbol] | [symbol] | [symbol] | [symbol] | [symbol] |
| 6 | 100 | 100 | 10000 | 5000 | 2 | 38 | 250 |
| 7 | 100 | 100 | 20000 | 10000 | 2 | 38 | 500 |
| 8 | 80 | 120 | 50000 | 20000 | 2 | 38 | 1000 |
| 9 | 100 | 100 | 100000 | 50000 | 2 | 38 | 2500 |
| 10 | — | — | — | 100000 | 2 | 38 | 5000 |

Stricken text in Table 10 of this Example indicates standard excluded from standard curve calculation due to low standard accuracy.

The linear curve had an intercept of 0.000653, slope of 0.00000996, and R² value of 0.9712. Quality control (QC) samples were analyzed at rimiducid concentrations of 20, 200, and 2000 ng/mL. The values are shown in Table 11 of this Example.

TABLE 11
QC Parameters
QC Samples

| | | Measured | |
|---|---|---|---|
| | Conc. | Conc. | Accuracy |
| Name | (ng/mL) | (ng/mL) | (%) |
| QC20 | 20 | 16.4 | 82 |
| QC200 | 200 | 172 | 86 |
| QC2000 | 2000 | 2750 | 138 |

### Results

Rimiducid plasma values and calculations are provided in Table 12 of this Example.

TABLE 12
Rimiducid Plasma Values and Calculations

| | | | | |
|---|---|---|---|---|
| Time (h) | Average | StDev | RSD | AUC |

sn-A (PEG400:H₂O:PS80, 30:40:30)

| | | | | | | | |
|---|---|---|---|---|---|---|---|
| 1 | 0.705 | 5.29 | 117 | 41.00 | 65.86 | 160.64 | 10395 |
| 2 | 203 | 27 | 37.3 | 89.10 | 98.77 | 110.86 | |
| 4 | 179 | 188 | 163 | 176.67 | 12.66 | 7.17 | |
| 8 | 216 | 298 | 325 | 279.67 | 56.77 | 20.30 | |
| 24 | 80.3 | 138 | 282 | 166.77 | 103.88 | 62.29 | |
| 48 | 403 | 155 | 294 | 284.00 | 124.30 | 43.77 | |

em-A (MCT:PEG400:H₂O:PS80, 30:15:40:15)

| | | | | | | | |
|---|---|---|---|---|---|---|---|
| 1 | 97.9 | 60.7 | 116 | 91.53 | 28.19 | 30.80 | 13488 |
| 2 | 118 | 96.3 | 76.9 | 97.07 | 20.56 | 21.18 | |
| 4 | 96.1 | 554 | 264 | 304.70 | 231.65 | 76.02 | |
| 8 | 213 | 430 | 525 | 389.33 | 159.93 | 41.08 | |
| 24 | 431 | 281 | 280 | 330.67 | 86.89 | 26.28 | |
| 48 | 95 | 347 | 15.5 | 152.50 | 173.07 | 113.49 | |

em-B (CO:H₂O:PS80, 30:60:10)

| | | | | | | | |
|---|---|---|---|---|---|---|---|
| 1 | 107 | 0 | 0 | 35.67 | 61.78 | 173.21 | 7501 |
| 2 | 65 | 99.1 | 50.8 | 71.63 | 24.82 | 34.65 | |
| 4 | 18.7 | 283 | 191 | 164.23 | 134.17 | 81.69 | |
| 8 | 61.7 | 95.7 | 328 | 161.80 | 144.93 | 89.58 | |
| 24 | 198 | 110 | 3.11 | 103.70 | 97.60 | 94.11 | |
| 48 | 27.8 | 267 | 247 | 180.60 | 132.71 | 73.48 | |

em-C (MCT:PEG400:H₂O:PS80, 30:15:40:15)

| | | | | | | | |
|---|---|---|---|---|---|---|---|
| 1 | 2.47 | 57.1 | 100 | 53.19 | 48.88 | 91.90 | 12366 |
| 2 | 101 | 169 | 24.4 | 98.13 | 72.34 | 73.72 | |
| 4 | 85.7 | 39.9 | 119 | 81.53 | 39.71 | 48.71 | |
| 8 | 169 | 232 | 237 | 212.67 | 37.90 | 17.82 | |
| 24 | 659 | 174 | 403 | 412.00 | 242.63 | 58.89 | |
| 48 | 33.5 | 168 | 187 | 129.50 | 83.68 | 64.62 | |

In Table 12 of this Example, ‘RSD’ indicates ‘Relative Standard Deviation’; ‘RSE’ indicates ‘Relative Standard Error’; plasma values are shown in ng/mL; area under the curve (AUC) values are in ng*h/mL.

Slow bleeds and dark blood were observed at various time points amongst animals in the various formulation dose groups. There was no observable pattern amongst the abnormal observations to attribute them to any formulation-related toxicities.

### Analysis

Pharmacokinetic (PK) analysis of rimiducid formulated in all four subcutaneous formulations resulted in extended exposure of rimiducid in rats, greater than threshold levels of about 50 ng/mL for induced caspase-9 apoptosis (e.g., ApoptiCIDe™) for at least 48 hours. In general, emulsion-based formulations performed slightly better than the rimiducid solution based on average AUC values of 11,118 ng*h/mL compared to 10,395 ng*h/mL, respectively.

### Example 3: Single Intramuscular Dose Pharmacokinetic Study (Non-GLP) of Rimiducid

Formulated in Emulsion-based Formulations Rimiducid was dosed by intramuscular (IM) administration to male CD-IGS rats in four emulsion-based formulations using oil, surfactant, and solvent excipients. The formulations were oil-in-water emulsions with a measurable particle size. The formulations tested were exploratory emulsion formulations. Emulsions were prepared by first mixing and forming a coarse emulsion. To form a fine emulsion, high-energy sonication was used to reduce particle size to facilitate increased bioavailability and long-term stability. The rimiducid utilized as the active ingredient was isomerically pure and had a structure according to Formula B herein.

Rats were bled using the retro-orbital bleeding (ROB) technique at 1, 2, 4, 8, 24, and 48 hours post-dosing. Following ROB bleeds, plasma was isolated from whole blood, and rimiducid was extracted from plasma and quantified by LCMS using a standard curve prepared with untreated rat plasma spiked with known concentrations of rimiducid.

Formulations were dosed to rats with a final dose volume of 0.2 mL due to the limited injection volume allowed by the IACUC for IM injection in rats. The formulations were prepared with a rimiducid concentration of 10 mg/mL, resulting in a rimiducid dose of approximately 6 mg/kg (see Table 5 of this Example). Any observed precipitation of rimiducid from the formulations is noted herein.

TABLE 1
Formulation Details

| Use Case | Formulation | Excipients | Volume Ratio |
|---|---|---|---|
| Emulsion | em-A | MCT/P400/H₂O/PS80 | 30/15/40/15 |
| Emulsion | em-B | CO/H₂O/PS80 | 30/60/10 |
| Emulsion | em-C | CO/P400/H₂O/PS80 | 30/15/40/15 |
| Emulsion | em-D | MCT/H₂O/PS80 | 30/60/10 |

In Table 1 of this Example, P400 is Polyethylene Glycol 400, PS80 is Polysorbate 80, MCT is Medium Chain Triglycerides, and CO is Castor Oil.

TABLE 2
Materials and Equipment

| | Description | Manuf. | Cat # |
|---|---|---|---|
| Reagents | | | |
| Rimiducid | Homodimerizer drug | MedChemExpress | Lot: 124865 |
| AP23510 | LCMS internal standard (IS) | MedChemExpress | HY-47001 |
| Methanol | Hypersolv, LCMS grade | VWR | BDH85800.400 |
| Water | Hypersolv, LCMS grade | VWR | BDH83645.400 |
| Formic Acid | HPLC grade | VWR | 97064-706 |
| Polyethylene Glycol 400 | Solvent excipient | VWR | AAB21992-30 |
| Polysorbate 80 | Surfactant excipient | VWR | BDH7781-2 |
| Medium Chain | Oil excipient, derived from coconut | Kroger | N/A |
| Triglycerides | oil | | |
| Castor Oil | Oil excipient, derived from seed of | Kroger | N/A |
| | the castor plant | | |
| Consumables | | | |
| Microcentrifuge tubes, 1.5- | 1.5-mL, snap cap | VWR | 76332-068 |
| mL | | | |
| Needle, disposable | 18 G, 1.5 in | BD | 305918 |
| Syringe, 5 mL | Slip tip | BD (VWR) | BD309647 |
| 22.18-mL Vials | Borosilicate glass, screw cap | VWR | 66011-143 |
| HPLC vials | Convenience kit with vial and | VWR | 89523-478 |
| | screw cap | | |
| HPLC vial inserts | 0.2 mL capacity | MicroSolv (VWR) | 97051-398 |
| Pipette tips - 1000 μL | Rainin BioClean Ultra, pre-racked, | Rainin | 30389212 |
| | with filter | | |
| Pipette tips - 200 μL | Rainin BioClean Ultra, pre-racked, | Rainin | 30389239 |
| | with filter | | |
| Pipette tips - 20 μL | Rainin BioClean Ultra, pre-racked, | Rainin | 17005860 |
| | with filter | | |

| Equipment | Manuf. | Model # | Serial # |
|---|---|---|---|
| Pipette - 2 μL | Rainin, Pipette-Lite LTS | 17014393 | |
| Pipette - 20 μL | Rainin, Pipette-Lite LTS | 17014392 | |
| Pipette - 200 μL | Rainin, Pipette-Lite LTS | 17014391 | |
| Pipette - 1000 μL | Rainin, Pipette-Lite LTS | 17014382 | |
| LCMS, triple quad | AB Sciex | 3200 | 45-1-1610- |
| | | | 2589-2 |
| Stir Bars | ½ inch magnetic stir bar | VWR | 58948-353 |
| LCMS Column | ThermoScientific Aquasil C18, | 77505-052130 | |
| | 2.1 × 50 mm, 5-μm pore size | | |
| High Energy Sonicator | Qsonica | Q500 | |
| Sonicator Microtip Probe, | Qsonica | 4418 | |
| ⅛″ tip | | | |

TABLE 3
Abbreviations

| Abbreviation | Definition |
|---|---|
| HPLC | High-performance liquid chromatography |
| IS | Internal Standard |
| IM | Intramuscular |
| LCMS | Liquid chromatography-mass spectrometry |
| Rim | rimiducid |
| RT | Room temperature |
| ROA | Route of administration |
| ROB | Retro-orbital bleed |
| RSD | Relative Standard Deviation |
| RSE | Relative Standard Error |
| SC | Subcutaneous |

TABLE 4
Study Design

| | |
|---|---|
| Product: | Rimiducid Formulation Development |
| Type of study: | Non-GLP Pharmacokinetic |
| Title of study: | Single Intramuscular Dose Pharmacokinetic Study (non-GLP) of Rimiducid |
| | Formulated in Emulsion-based Formulations |
| Objectives: | There is one main objective in this experiment: |
| | To test the intramuscular bioavailability of rimiducid formulated in emulsion-based |
| | formulations in CD-IGS rats: |
| | em-A - MCT/P400/H₂O/PS80, 30/15/40/15 v/v/v/v |
| | em-B - CO/H₂O/PS80, 30/60/10 v/v/v |
| | em-B - CO/P400/H₂O/PS80, 30/15/40/15 v/v/v |
| | em-D - MCT/H₂O/PS80, 30/60/10 |
| Animal | Charles River |
| Vendor/CRO: | |
| Species and | CD-IGS Rats |
| strain: | |
| Species sex: | Male |
| Species age or | 300 grams |
| weight at | |
| dosing: | |
| Test article(s): | Rimiducid - MCT/P400/H₂O/PS80, 30/15/40/15 v/v/v/v (em-A) |
| | Rimiducid - CO/H₂O/PS80, 30/60/10 v/v/v (em-B) |
| | Rimiducid - CO/P400/H₂O/PS80, 30/15/40/15 v/v/v (em-C) |
| | Rimiducid - MCT/H₂O/PS80, 30/60/10 v/v/v (em-D) |
| Particle size | em-A - Z-ave - 1229 nm, Pdl - 1 |
| details: | em-B - Z-ave - 669 nm, Pdl -0.44 |
| | em-C - Z-ave - 185 nm, Pdl - 0.02 |
| | em-D - Z-ave - 149 nm, Pdl - 0.12 |
| Study design: | Rimiducid esd intramuscularly (IM) administered to CD-IGS rats (n = 3 per group) |
| | based on Table 5 of this Example. The maximum dose for IM injections in rats was |
| | limited to 0.1 mL per site, two sites will be injected (left and right flank regions) for a |
| | total dose volume of 0.2 mL. |
| | Rats were monitored for any signs of toxicity or abnormal behavior. |
| | Time points of blood collection post-dosing were as follows (h): 1, 2, 4, 8, 24, 48 |
| Dose, dose | Rats were dosed IM with a total dose volume of 0.2 mL, which amounts to an |
| justification and | average rimiducid dose of about 6 mg/kg. Refer to Table 5 of this Example for the |
| route of | rimiducid dose per group based on average animal weight per group. This was the |
| administration | maximum rimiducid dose with the current formulations, limited by the total volume |
| (ROA): | for IM injections in rats. |
| Outcome | Endpoints: |
| measures and | Rats were bled using the retro-orbital bleeding technique (ROB) at specified time |
| endpoints: | points based on Table 5 of this Example. |
| | Plasma concentrations of rimiducid were measured using pre-determined LCMS |
| | methods compared to known standards. |
| | Rats were monitored for any signs of toxicity or abnormal behavior after dosing of |
| | rimiducid in the new formulations. |
| Study duration: | 3 Days |

### Route of Drug Administration (ROA)

Rats received a single dose of the drug formulation via IM administration.

### Blood Collection

ROB was performed on rats in each group at pre-determined time intervals (see Table 5 of this Example). Following ROB at the terminal time point, rats were euthanized. Blood samples were collected into EDTA-coated (purple top) tubes at specified time points post-drug administration (see Table 5 of this Example).

### Clinical Observations

Rats were observed for adverse clinical signs after dosing the formulations.
General—nutritional state, quality of hair and coat, behavioral features, and locomotor abnormalities
Head—hair coverage, any abnormal respiratory sounds, discoloration or abnormal discharge from the mouth, position/size of eyeball, discoloration or fluid discharge from eyeball, abnormalities in ear positions or discharge from ear
Neck—mobility, shape, any wounds/masses, guttural sounds
Trunk—position, symmetry, and size of back, thorax, abdomen, or pelvis. Any abnormal movements or respiratory sounds/movements will be noted
Limbs/tail—position, motility, abnormal muscle, proper use (versus lameness).

TABLE 5
Experimental Groups

| | | Dose | Actual API | | | | |
|---|---|---|---|---|---|---|---|
| | | Volume | Dose | | | | Number |
| Group | Formulation | (mL) | (mg/kg) | ROA | Fasting | Time Points (h) | of Rats |
| 1 | em-A | 0.2 | 6.1 | IM | N | 1, 2, 4, 8, 24, 48 | 3 |
| 2 | em-B | 0.2 | 6.3 | IM | N | 1, 2, 4, 8, 24, 48 | 3 |
| 3 | em-C | 0.2 | 6.4 | IM | N | 1, 2, 4, 8, 24, 48 | 3 |
| 4 | em-D | 0.2 | 6.3 | IM | N | 1, 2, 4, 8, 24, 48 | 3 |

### Dose Formulation Preparation (Emulsions)

TABLE 6
Activities

| Date | Activity |
|---|---|
| Day 0 | Body weights |
| Day 0 | Drug administration |
| Day 0-Day 2 | Blood collection |
| Day 0-Day 2 | Plasma extraction from whole blood |
| Day 2 | Plasma delivered to Ponce Therapeutics or |
| | bioanalysis |
| Day 2 | Plasma samples received |
| Day 8-Day 9 | LCMS bioanalysis |

Rimiducid was weighed into 20-mL glass vials and weights were recorded. The final target concentrations for all rimiducid IM formulations were 10 mg rimiducid per mL of formulation. Hydrophobic components (i.e., oils (CO, MCT)) were combined with rimiducid and stirred while heating at 60° C. until the rimiducid was dissolved. Stirring/heating took place for a maximum of 30 minutes. The next steps were performed even if rimiducid was not dissolved in solution (i.e., precipitates were observed).

PS80 was dissolved in water and heated to 60° C. while stirring to form an aqueous mixture. When P400 solvent was included, it was mixed with PS80 in water to form an aqueous mixture. After the PS80, and P400 when included, completely dissolved in the water, the aqueous mixture was slowly added to the oil/rimiducid mixture while stirring under heating at 60° C. The vials were heated to 60° C. and stirred with a magnetic stir bar for 2 hours. The coarse emulsions (i.e., emulsions that had not been sonicated) were left on the bench top overnight to cool to room temperature.

The following day, coarse emulsions were observed for phase separation and rimiducid precipitation. Those coarse emulsions that were phase separated were sonicated. Those coarse emulsions where rimiducid precipitation was observed were excluded from further development.

Coarse emulsions were subjected to sonication after stir bar removal for 5 minutes at room temperature, using a 4/2 second on/off pulse pattern and 25% amplitude. A 1/8″ microtip probe was used as the formulation volume was small and sonication was carried out in the same 20 mL glass vials in which the coarse emulsions were mixed.

Particle-size analysis was then carried out on the emulsion formulations by diluting the emulsions 1:100 in pure water and measuring the emulsions using a Zetasizer instrument. Refer to Tables 7 and 8 of this Example for formulation and particle-size details.

TABLE 7
IM Formulation Excipient Details

| | | | Rimiducid | | | Stable |
|---|---|---|---|---|---|---|
| | | Ratio | Conc. | Sonication | Precipitation | Emulsion |
| Formulation | Components | (vol %) | (mg/mL) | (Y/N) | (Y/N) | (Y/N) |
| sn-A | P400/H₂O/ | 30/40/ | 10 | N | N | N/A, solution |
| | PS80 | 30 | | | | |
| em-A | MCT/P400/ | 30/15/ | 10 | N | N | Y, no |
| | H₂O/PS80 | 40/15 | | | | sonication |
| | | | | | | required |
| em-B | CO/H₂O/ | 30/60/ | 10 | Y | N | Y, only after |
| | PS80 | 10 | | | | sonication |
| em-C | CO/P400/ | 30/15/ | 10 | Y | N | Y, only after |
| | H₂O/PS80 | 04/15 | | | | sonication |
| em-D | MCT/H₂O/ | 30/60/ | 10 | Y | Initially, all API | Y, only after |
| | PS80 | 10 | | | dissolved after | sonication |
| | | | | | stirring at 80° C. | |
| N/A | SO/H₂O/ | 30/60/ | 10 | N | Y, High | N |
| | PS80 | 10 | | | amount | |
| N/A | CA/H₂O/PS80 | 30/60/ | 10 | N | Y, High | N |
| | | 10 | | | amount | |
| N/A | CA/P400/H₂O/ | 27/14/36/ | 10 | Y | N | Y, only after |
| | PS80/SP80 | 14/9 | | | | sonication |

CO = Castor Oil
SO = Sesame Oil
MCT = Medium Chain Triglycerides
CA = Canola Oil
PS80 = Polysorbate 80
SP80 = Span 80
P400 = PEG400
H2O = Purified Water

Only formulations em-A, em-B, em-C and em-D were dosed in this study. The bottom three formulations listed in Table 7 of this Example were not considered viable formulations due to high amounts of precipitation, unstable emulsions, or high viscosities.

TABLE 8
Emulsion Particle Size Details

| | | | Particle | | | | | |
|---|---|---|---|---|---|---|---|---|
| | | | Diameter | Average | | | | |
| | | Ratio | (Z-avg, | Z-avg | StDev | | Pdl | Pdl |
| Formulation | Components | (vol %) | nm) | (nm) | (nm) | Pdl | Avg | StDev |
| em-A | MCT/P400/ | 30/15/ | 1234 | 1228.67 | 10.12 | 1 | 1.00 | 0.00 |
| | H₂O/PS80 | 40/15 | 1217 | | | 1 | | |
| | | | 1235 | | | 1 | | |
| em-B | CO/H₂O/ | 30/60/10 | 643.3 | 668.37 | 22.47 | 0.9 | 0.44 | 0.42 |
| | PS80 | | 675.1 | | | 0.336 | | |
| | | | 686.7 | | | 0.084 | | |
| em-C | CO/P400/ | 30/15/ | 187.9 | 185.03 | 2.85 | 0.033 | 0.02 | 0.02 |
| | H₂O/PS80 | 40/15 | 185 | | | 0.007 | | |
| | | | 182.2 | | | 0.007 | | |
| em-D | MCT/H₂O/PS80 | 30/60/10 | 150.3 | 148.60 | 1.96 | 0.136 | 0.12 | 0.02 |
| | | | 149 | | | 0.134 | | |
| | | | 146.5 | | | 0.099 | | |

Pdl—Polydispersity

### LCMS Analysis of In Vivo Plasma Samples

LCMS analysis was performed on plasma samples from rats. Plasma concentrations of rimiducid were quantified using the LC/MS method described in Table 9 of this Example (binary gradient of water with 0.1% formic acid and methanol with 0.1% formic acid).

TABLE 9
LCMS Parameters

| Time | % | % | | |
|---|---|---|---|---|
| (minutes) | Water | Methanol | Mass Peaks | |
| 0.00 | 30 | 70 | Rimiducid | Q1: 1419.93 |
| 0.00 | 30 | 70 | | Q3: 151.10 |
| 2.00 | 0 | 100 | AP23510 (IS) | Q1: 1433.95 |
| 2.01 | 0 | 100 | | Q3: 151.00 |
| 3.00 | 30 | 70 | | |
| 5.00 | 30 | 70 | | |

A standard curve was prepared by diluting rimiducid in DMSO and spiking into naïve rat plasma, following Table 10 of this Example. The standards were analyzed, and a linear standard curve was calculated by the LCMS software using area under the curve as the parameter and 1/x² weighting. The ratio of AP1903 Peak Area to IS Peak Area (x-axis) was correlated to AP1903 concentration (y-axis). For sample analysis, 100 μL of plasma isolated from whole blood was added to 100 μL of acetonitrile containing 2.0 μg/mL AP23510 (used as the internal standard). The samples were vortexed and then centrifuged for 10 minutes at 12,000×g to extract rimiducid from rat plasma. Rimiducid concentrations in plasma samples were calculated using the standard curve.

TABLE 10
LCMS Standards

| | | | | | Matrix Standards | | |
|---|---|---|---|---|---|---|---|
| | | | | | | Blank | |
| | 20 × Standard Stocks | | | | | Matrix | |
| | Stock | | | DMSO | Vol- | (plasma | |
| | Vol- | | Stock | Stock | ume | from | |
| Std | ume | DMSO | used | Conc | Stock | untreated | Conc. |
| # | (μL) | (μL) | (μL) | (ng/mL) | (μL) | rats) | (ng/mL) |
| 1 | 100 | 100 | 200 | 100 | 2 | 38 | 5 |
| 2 | 80 | 120 | 500 | 200 | 2 | 38 | 10 |
| 3 | 100 | 100 | 1000 | 500 | 2 | 38 | 25 |
| 4 | 100 | 100 | 2000 | 1000 | 2 | 38 | 50 |
| 5 | 80 | 120 | 5000 | 2000 | 2 | 38 | 100 |
| 6 | 100 | 100 | 10000 | 5000 | 2 | 38 | 250 |
| 7 | 100 | 100 | 20000 | 10000 | 2 | 38 | 500 |
| 8 | 80 | 120 | 50000 | 20000 | 2 | 38 | 1000 |
| [symbol] | [symbol] | [symbol] | [symbol] | [symbol] | [symbol] | [symbol] | [symbol] |
| [symbol] | — | — | — | [symbol] | [symbol] | [symbol] | [symbol] |

Stricken text in Table 10 of this Example indicates standard excluded from standard curve calculation due to low standard accuracy.

The linear curve had an intercept of 0.000258, slope of 0.0000572, and R² value of 0.9479. Quality control (QC) samples were analyzed at rimiducid concentrations of 20, 200, and 2000 ng/mL. The values are shown in Table 11 of this Example.

TABLE 11
QC Parameters
QC Samples

| | | Measured | |
|---|---|---|---|
| | Conc. | Conc. | Accuracy |
| Name | (ng/mL) | (ng/mL) | (%) |
| QC20 | 20 | 19 | 95 |
| QC200 | 200 | 226 | 113 |
| QC2000 | 2000 | 1950 | 103 |

### Results

Rimiducid plasma values and calculations are provided in Table 12 of this Example.

TABLE 12
Rimiducid Plasma Values and Calculations

| | | | | |
|---|---|---|---|---|
| Time (h) | Average | StDev | RSD | AUC |

em-A (MCT:PEG400:H₂O:PS80, 30:15:40:15)

| | | | | | | | |
|---|---|---|---|---|---|---|---|
| 1 | 69.8 | 79.7 | 57.5 | 69.00 | 11.12 | 16.12 | 5842 |
| 2 | 54.8 | 45.1 | 58.3 | 52.73 | 6.84 | 12.97 | |
| 4 | 57.6 | 53.2 | 109 | 73.27 | 31.02 | 42.34 | |
| 8 | 233 | 121 | 303 | 219.00 | 91.80 | 41.92 | |
| 24 | 314 | 110 | 58.6 | 160.87 | 135.08 | 83.97 | |
| 48 | 5.29 | 7.17 | 4.28 | 5.58 | 1.47 | 26.28 | |

em-B (CO:H₂O:PS80, 30:60:10)

| | | | | | | | |
|---|---|---|---|---|---|---|---|
| 1 | 22.8 | 31.5 | 36.5 | 30.27 | 6.93 | 22.91 | 7672 |
| 2 | 37.8 | 45.2 | 48.3 | 43.77 | 5.39 | 12.33 | |
| 4 | 11.1 | 61.6 | 252 | 108.23 | 127.04 | 117.38 | |
| 8 | 420 | 391 | 138 | 316.33 | 155.12 | 49.04 | |
| 24 | 80 | 288 | 195 | 187.67 | 104.19 | 55.52 | |
| 48 | 33.7 | 24.7 | 25.2 | 27.87 | 5.06 | 18.15 | |

em-C (CO:PEG400:H₂O:PS80, 30:15:40:15)

| | | | | | | | |
|---|---|---|---|---|---|---|---|
| 1 | 33.5 | 46.8 | 49.3 | 43.20 | 8.49 | 19.66 | 7234 |
| 2 | 109 | 50.1 | 74.2 | 77.77 | 29.61 | 38.08 | |
| 4 | 152 | 61 | 99.7 | 104.23 | 45.67 | 43.81 | |
| 8 | 226 | 105 | 397 | 242.67 | 146.71 | 60.46 | |
| 24 | 136 | 124 | 158 | 139.33 | 17.24 | 12.38 | |
| 48 | — | 214 | 44 | 129.00 | 120.21 | 93.18 | |

em-D (MCT:H₂O:PS80, 30:60:10)

| | | | | | | | |
|---|---|---|---|---|---|---|---|
| 1 | 23.6 | 47.2 | 94.1 | 54.97 | 35.89 | 65.29 | 7712 |
| 2 | 305 | 366 | 113 | 261.33 | 132.03 | 50.52 | |
| 4 | 165 | 413 | 306 | 294.67 | 124.39 | 42.21 | |
| 8 | 511 | 344 | 305 | 386.67 | 109.43 | 28.30 | |
| 24 | 85.6 | 46.3 | 164 | 98.63 | 59.92 | 60.75 | |
| 48 | 34.7 | 30.6 | 70 | 45.10 | 21.66 | 48.03 | |

In Table 12 of this Example, ‘RSD’ indicates ‘Relative Standard Deviation’; ‘RSE’ indicates ‘Relative Standard Error’; plasma values are shown in ng/mL; area under the curve (AUC) values are in ng*h/mL; and ‘-’ indicates no time point was taken and no plasma value was obtained.

Rats in group 3 displayed clinical observations such as slow bleeding, slow recovery from anesthesia, and dark blood. Rat number 1 from Group 3 died unexpectedly sometime between the 24- and 48-hour timepoints. All rats from group 3 presented the symptoms described above, and no other groups presented these symptoms. The formulation dosed to group 3 rats (em-C) potentially caused the adverse event.

### Analysis

Pharmacokinetic (PK) analysis of rimiducid formulated in all four intramuscular formulations resulted in extended exposure of rimiducid in rats, greater than required threshold levels of approximately 50 ng/mL for cellular apoptosis by an inducible caspase-9 (e.g., ApoptiCIDe™) for at least 24 hours. In general, the bioavailability of the formulations dosed IM, compared to SC (see Example 2), was higher overall. This higher bioavailability is most likely due to the more highly vascularized muscle tissue compared to the SC region. However, it may also be that the exposure window of the formulations dosed SC is longer than IM.

### Example 4: Preparation and Analysis of Predominantly Polar Solvent Formulations

This Example describes a process for preparing and assessing predominantly polar solvent pharmaceutical composition formulations containing the active ingredient rimiducid (i.e., having a structure according to Formula B). The predominantly polar solvent pharmaceutical composition formulations are for administration by injection (e.g., intramuscular administration; subcutaneous administration).

Rimiducid in an amount of 0.2 grams was weighed into a glass vial. A single polar solvent in an amount of 1 gram then was added to the glass vial. The polar solvent added to each vial was N-methyl 2-pyrrolidone (NMP), polyethylene glycol 300 (PEG300), polyethylene glycol 400 (PEG400), dimethyl sulfoxide (DMSO), 2-(2-ethoxyethoxy)ethanol (EEE) or ethanol. The EEE solvent utilized was commercially available under the tradename Transcutol®. Rimiducid and solvents each were at least 90% pure. A stir bar was added and the rimiducid and polar solvent was stirred under heating at 50 degrees Celsius for approximately three hours. The vial then was removed from heat and stirred for about 21 hours at room temperature. After stirring for a total time of about 24 hours, the formulation was quantified for rimiducid content using high performance liquid chromatography (HPLC). The formulation was clarified prior to preparing the formulation sample for analysis by HPLC (e.g., centrifuged at 6000×g for 10 minutes or filtered through a 0.22 um sterile filtration unit) to remove any precipitated or aggregated rimiducid. The clarified rimiducid formulation was diluted 1:1000 in sample diluent (40:60 v:v water:acetonitrile), vortexed, and injected onto the HPLC instrument for quantitation using a standard curve of known rimiducid concentrations. Rimiducid content in both the analyte and the standards was monitored by ultra-violet (UV) detection at 275 nm. Rimiducid content was determined according to a weight-to-weight (wt/wt) concentration of milligrams (mg) rimiducid to total gram weight of the formulation (mg/g). Analysis of the single polar solvent formulations resulted in concentrations (wt/wt) of soluble rimiducid in PEG400, PEG300, DMSO, NMP, ethanol, and EEE of 191.3 mg/g, 188.7 mg/g, 143.9 mg/g, 143.8 mg/g, 164.3 mg/g, and 119.7 mg/g, respectively.

Polar solvent pharmaceutical composition formulations containing the active ingredient and two cosolvents (binary mixture), three co-solvents (ternary mixture), or four cosolvents (quaternary mixture) are prepared and assessed as described in the foregoing paragraph, except that in place of a single polar solvent, multiple polar co-solvents are mixed, and then added to rimiducid in a glass vial. The following are cosolvent formulation candidates for preparation and assessment.

Amounts are percent volume of each cosolvent to overall volume of the pharmaceutical composition formulation (% v/v). The percent volume of the cosolvents for each pharmaceutical composition formulation sums to 100%. Solvents each typically are at least 90% pure.

### Binary Solvent Formulation (Containing Two Co-Solvents)

1. PEG300/NMP: 1-20% (v/v) PEG300 and 80-99% (v/v) NMP
2. PEG400/NMP: 1-50% (v/v) PEG400 and 50-99% (v/v) NMP
3. NMP/DMSO: 90-99% (v/v) NMP and 1-10% (v/v) DMSO
4. NMP/Ethanol: 94-99% (v/v) NMP and 1-6% (v/v) ethanol

### Ternary Solvent Formulation (Containing Three Co-Solvents)

1. NMP/DMSO/Ethanol: 10-80% (v/v) NMP, 1-15% (v/v) DMSO and 1-15% (v/v) ethanol
2. NMP/Ethanol/PEG300: 10-80% (v/v) NMP, 1-15% (v/v) ethanol and 1-20% (v/v) PEG300
3. NMP/Ethanol/PEG400: 10-80% (v/v) NMP, 1-15% (v/v) ethanol and 1-50% (v/v) PEG400

### Quaternary Solvent Formulation (Containing Four Co-Solvents)

1. PEG300/NMP/DMSO/Ethanol: 1-20% (v/v) PEG300, 10-80% (v/v) NMP, 1-15% (v/v) DMSO and 1-15% (v/v) ethanol
2. PEG400/NMP/DMSO/Ethanol: 1-50% (v/v) PEG400, 10-40% (v/v) NMP, 1-15% (v/v) DMSO and 1-15% (v/v) ethanol

### Example 5: Preparation and Analysis of Predominantly Oil Formulations

This Example describes a process for preparing and assessing predominantly oil pharmaceutical composition formulations containing the active ingredient rimiducid (i.e., having a structure according to Formula B) and an oil component. The predominantly oil pharmaceutical composition formulations are for administration by injection (e.g., intramuscular administration; subcutaneous administration).

Rimiducid in an amount of 0.2 grams was weighed into a glass vial. The oil in the amount of 1 gram then was added to the glass vial. The oil was highly refined polyethoxylated castor oil (PECO; commercially available under the tradename Cremophor EL®). The oil and rimiducid each were at least 90% pure. A stir bar was added and the rimiducid and oil was stirred under heating at 50 degrees Celsius for approximately three hours. The vial then was removed from heat and stirred for about 21 hours at room temperature. After stirring for a total time of about 24 hours, the formulation was quantified for rimiducid content using high performance liquid chromatography (HPLC). The formulation was clarified prior to preparing the formulation sample for analysis by HPLC (e.g., centrifuged at 6000×g for 10 minutes or filtered through a 0.22 um sterile filtration unit) to remove any precipitated or aggregated rimiducid. The clarified rimiducid formulation was diluted 1:1000 in sample diluent (40:60 v:v water:acetonitrile), vortexed, and injected onto the HPLC instrument for quantitation using a standard curve of known rimiducid concentrations. Rimiducid content in both the analyte and the standards was monitored by ultra-violet (UV) detection at 275 nm. Rimiducid content was determined according to a weight-to-weight (wt/wt) concentration of milligrams (mg) rimiducid to total gram weight of the formulation (mg/g). Analysis of the predominantly oil formulation reported a concentration (wt/wt) of soluble rimiducid of 110.1 mg/g. Additional predominantly oil pharmaceutical composition formulations in which the oil described above, PECO, is replaced by castor oil or cottonseed oil, can be prepared as described.

### Example 6: Preparation and Analysis of Predominantly Oil Formulations Containing a Polar Solvent

This Example describes a process for preparing and assessing a predominantly oil pharmaceutical composition formulation containing the active ingredient rimiducid (i.e., having a structure according to Formula B), an oil, at least one polar solvent. Rimiducid, oil and solvents each are at least 90% pure. Rimiducid is weighed into a glass vial and mass is recorded. A known volume of polar solvent (i.e., single solvent formulation), or mixture of polar cosolvents (binary, ternary, quaternary cosolvent formulation) is then added to the glass vial. The single solvent or cosolvent mixture utilized can be any single solvent or co-solvent mixture described in Example 4 or described in this Example. A stir bar is added and the rimiducid and polar solvent or mixture of polar solvents is stirred under heating at 50 degrees Celsius for about one hour. A known volume of an oil then is added to a final concentration of 50% v/v or greater and the mixture further stirred under heating at 50 degrees Celsius for about two additional hours. Castor oil, cottonseed oil, and polyethoxylated castor oil (PECO; commercially available under the tradename Cremophor EL®) are candidate oils for preparation and assessment. The vial then is removed from heat and stirred for about 21 hours at room temperature. After stirring for a total of about 24 hours, the formulation is quantified for rimiducid content using high performance liquid chromatography (HPLC). Prior to preparing the formulation sample for HPLC analysis, the formulation is clarified (e.g., centrifuged at 6000×g for 10 minutes or filtered through a 0.22 um sterile filtration unit) to remove any precipitated or aggregated rimiducid. The clarified rimiducid formulation is diluted 1:1000 in sample diluent (40:60 v:v water:acetonitrile), vortexed, and injected onto the HPLC instrument for quantitation using a standard curve of known rimiducid concentrations. Rimiducid content in both the analyte and the standards is monitored by ultra-violet (UV) detection at 275 nm. Rimiducid content is determined according to a weight-to-weight (wt/wt) concentration of milligrams (mg) rimiducid to total gram weight of the formulation (mg/g).

The following are predominantly oil formulation candidates for preparation and assessment utilizing the process described in this Example. Amounts are percent volume of solvent to overall volume of the pharmaceutical composition formulation (% v/v). The percent volume of the oil and solvent components for each pharmaceutical composition formulation sums to 100%. Solvents each are at least 90% pure. The “Oil” in the following pharmaceutical composition formulations is castor oil, cottonseed oil or polyethoxylated castor oil (PECO, commercially available under the tradename Cremophor EL®).

### Predominantly Oil and One Polar Solvent (Containing One Oil and One Co-Solvent)

1. Oil/PEG300: 80-99% (v/v) Oil and 1-20% (v/v) PEG300
2. Oil/PEG400: 51-99% (v/v) Oil and 1-49% (v/v) PEG400
3. Oil/NMP: 51-99% (v/v) Oil and 1-49% (v/v) NMP
4. Oil/DMSO: 90-99% (v/v) Oil and 1-10% (v/v) DMSO
5. Oil/Ethanol: 94-99% (v/v) NMP and 1-6% (v/v) ethanol.

### Predominantly Oil and Two Polar Solvents (Containing One Oil and Two Co-Solvents)

1. Oil/PEG300/NMP: 50-98% (v/v) Oil and 1-20% (v/v) PEG300 and 1-40% (v/v) NMP
2. Oil/PEG400/NMP: 50-98% (v/v) Oil and 1-20% (v/v) PEG400 and 1-40% (v/v) NMP
3. Oil/NMP/DMSO: 50-98% (v/v) Oil and 1-40% (v/v) NMP and 1-10% (v/v) DMSO
4. Oil/NMP/Ethanol: 50-98% (v/v) Oil and 1-40% (v/v) NMP and 1-6% (v/v) ethanol
5. Oil/PEG300/DMSO: 50-98% (v/v) Oil and 1-20% (v/v) PEG300 and 1-10% (v/v) DMSO
6. Oil/PEG400/DMSO: 50-98% (v/v) Oil and 1-20% (v/v) PEG400 and 1-10% (v/v) DMSO
7. Oil/PEG300/Ethanol: 50-98% (v/v) Oil and 1-20% (v/v) PEG300 and 1-6% (v/v) ethanol
8. Oil/PEG400/Ethanol: 50-98% (v/v) Oil and 1-20% (v/v) PEG400 and 1-6% (v/v) ethanol.

### Predominantly Oil and Three Polar Solvents (Containing One Oil and Three Co-Solvents)

1. Oil/NMP/DMSO/Ethanol: 50-97% (v/v) Oil and 1-40% (v/v) NMP, 1-10% (v/v) DMSO and 1-6% (v/v) ethanol
2. Oil/NMP/Ethanol/PEG300: 50-97% (v/v) Oil and 1-40% (v/v) NMP, 1-6% (v/v) ethanol and 1-20% (v/v) PEG300
3. Oil/NMP/Ethanol/PEG400: 50-97% (v/v) Oil and 1-40% (v/v) NMP, 1-6% (v/v) ethanol and 1-50% (v/v) PEG400
5. Oil/PEG300/DMSO/Ethanol: 50-97% (v/v) Oil and 1-20% (v/v) PEG300, 1-10% (v/v) DMSO and 1-6% (v/v) ethanol
6. Oil/PEG400/DMSO/Ethanol: 50-97% (v/v) Oil and 1-50% (v/v) PEG300, 1-10% (v/v) DMSO and 1-6% (v/v) ethanol.

### Predominantly Oil and Four Polar Solvents (Containing One Oil and Four Co-Solvents)

1. Oil/PEG300/NMP/DMSO/Ethanol: 50-96% (v/v) Oil and 1-20% (v/v) PEG300, 1-40% (v/v) NMP,
1-15% (v/v) DMSO and 1-6% (v/v) ethanol
2. Oil/PEG400/NMP/DMSO/Ethanol: 50-96% (v/v) Oil and 1-20% (v/v) PEG400, 1-40% (v/v) NMP,
1-15% (v/v) DMSO and 1-6% (v/v) ethanol.

### Example 7: Transdermal Rimiducid Formulations and Patches

Rimiducid (having a structure according to Formula B) is formulated for incorporation into, and administration from, transdermal patches. Transdermal delivery provides certain advantages over oral and subcutaneous routes of administration. For example, some patients have an extreme aversion to needles and are uncomfortable receiving SC injection, no matter how painless or easy they are to administer. Some patients, such as geriatric or pediatric, have difficulty swallowing pills or cannot swallow pills. Thus, formulation of rimiducid for transdermal delivery provides a flexible route of administration and expands the patient population to which rimiducid, and those therapies depending on rimiducid dosing, can be delivered.

Formulation of rimiducid in a solubilizing excipient component containing a solvent is conducted, followed by solubility screening of rimiducid. Solvents are selected from FDA's list of approved inactive ingredients.

### Transdermal Excipient Solubility Screening

Solvents shown in Table 1 of this Example are formulated with rimiducid are prepared and sonicated until rimiducid has dissolved completely. The excipient 2-(2-ethoxyethoxy)ethanol (EEE), having formula CH₃CH₂OCH₂CH₂OCH₂CH₂OH), is commercially available under the tradename Transcutol®.

TABLE 1
Transdermal Excipient

Ethyl Acetate
Ethanol
Isopropyl myristate (IPM)
Dimethyl isosorbide (DMI)
2-(2-ethoxyethoxy)ethanol (EEE; Transcutol ®)
Caprylic/capric triglycerides (CCT)
Ethyl acetate/Ethanol (50/50, v/v)
IPM/Ethanol (50/50, v/v)
Ethyl acetate/Ethanol/DMI/Transcutol ® (25/25/15/35, v/v/v/v)
IPM/Ethanol/Transcutol ®/DMI (35/15/35/15, v/v/v/v)
CCT/Ethanol/DMI/Transcutol ®/PG (25/25/15/30/5, v/v/v/v/v)
IPM/PG/Transcutol ®/DMI (40/10/35/15, v/v/v/v)
CCT/Ethanol/Transcutol ®/DMI (35/15/35/15, v/v/v/v)
Ethyl Acetate/IPM/Ethanol/DMI/Transcutol ®(15/15/25/15/30, v/v/v/v)

The formulations are tested for solubility (e.g., greater than 20 milligrams of rimiducid per 1 gram of excipient; greater than 50 milligrams of rimiducid per 1 gram of excipient; greater than 100 milligrams of rimiducid per 1 gram of excipient) and then for maximal potential skin permeation. A static Franz-Cell diffusion apparatus, which is a dermatomal human cadaver skin model (see, World Wide Web URL permegear.com/franz-cells/ear) is utilized to assess skin permeation of the rimiducid transdermal formulations. Using such an apparatus, transdermal rimiducid formulations, having different concentrations of rimiducid, are applied to cadaver skin (membrane) on one side of the chamber and media is collected on the other side of the skin (via a sampling port). The amount of rimiducid that transverses the skin tissue (rimiducid flux) is quantified, and total flux of rimiducid through the skin is determined. Samples are collected for up to 48 hours after transdermal administration of rimiducid to skin. Following quantification, the total amount of rimiducid that has transversed the skin is calculated by determining the area under the curve (AUC) of the transdermally delivered rimiducid and compared to the total rimiducid dose at the beginning of the study. Transepidermal electrical resistance (TEER) measurements verify skin barrier integrity of the tissue used in the skin absorption study. In optimized transdermal formulations, 25-50% of the total rimiducid dosed to the cadaver skin can permeate, and total flux can range from 100 to 2500 nanograms per milliliter over a time period of about 24 hours.

A formulation containing the following amounts of rimiducid and additives is tested for a transdermal route of administration: isopropyl myristate (20-50 wt %), ethanol (5-25 wt %, Transcutol® (25-50 wt %), and dimethyl isosorbide (5-25 wt %) and rimiducid (2-10 wt %).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents are based on available information and are not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. A pharmaceutical composition or component therein “consisting essentially of” a particular agent (“agent x”) can include one or more other agents (e.g., including “agent y”) that do not materially affect the basic and novel characteristics of agent x. Other agents (e.g., including “agent y”) that do not materially affect the basic and novel characteristics of agent x typically alter the solubility of an active ingredient (e.g., rimiducid, rimiducid family compound or rimiducid analog) in the pharmaceutical composition by 5% or less.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described, the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

1-33. (canceled)

34. A pharmaceutical composition, comprising:
a compound of Formula A, or a pharmaceutically acceptable salt thereof: 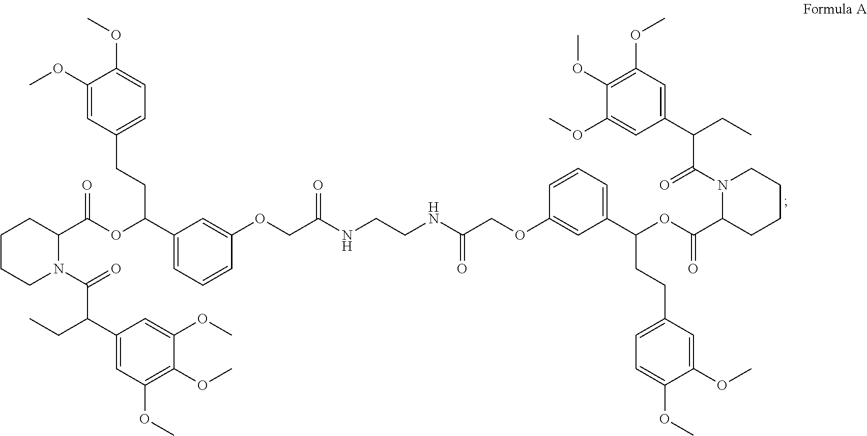 [SMILES: CCC(C(=O)N1CCCCC1C(=O)OC(CCc1ccc(OC)c(OC)c1)c1cccc(OCC(=O)NCCNC(=O)COc2cccc(C(CCc3ccc(OC)c(OC)c3)OC(=O)C3CCCCN3C(=O)C(CC)c3cc(OC)c(OC)c(OC)c3)c2)c1)c1cc(OC)c(OC)c(OC)c1]
and
an oil component.

35. The pharmaceutical composition of claim 34, wherein the oil component comprises medium-chain triglycerides (MCT), castor oil (CO), and/or polyethoxylated castor oil (PECO).

36. The pharmaceutical composition of claim 34, wherein the oil component is about 5% to about 50% by volume in the pharmaceutical composition or is about 25% to about 35% by volume in the pharmaceutical composition.

37. The pharmaceutical composition of claim 34, further comprising a surfactant component.

38. The pharmaceutical composition of claim 37, wherein the surfactant comprises a polysorbate.

39. The pharmaceutical composition of claim 37, wherein the surfactant comprises polysorbate 80 (PS80).

40. The pharmaceutical composition of claim 37, wherein the surfactant comprises a polysorbate and the oil component comprises medium-chain triglycerides (MCT), castor oil (CO), and/or polyethoxylated castor oil (PECO).

41. The pharmaceutical composition of claim 37, wherein the surfactant consists of polysorbate 80 (PS80) and the oil component consists of medium-chain triglycerides (MCT) and polyethoxylated castor oil (PECO).

42. The pharmaceutical composition of claim 34, further comprising a polar solvent component.

43. The pharmaceutical composition of claim 42, wherein the polar solvent component comprises ethanol.

44. The pharmaceutical composition of claim 34, wherein the oil component comprises a fatty acid.

45. The pharmaceutical composition of claim 44, wherein the fatty acid comprises a C8 fatty acid.

46. The pharmaceutical composition of claim 34, wherein the oil component comprises medium-chain triglycerides (MCT), polyethoxylated castor oil (PECO), and at least one fatty acid.

47. The pharmaceutical composition of claim 34, wherein the compound is of Formula B, or a pharmaceutically acceptable salt thereof:

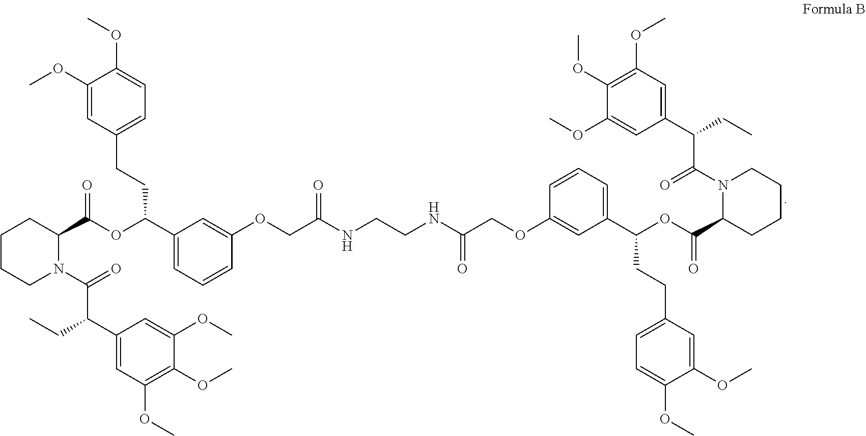 [SMILES: CC[C@H](C(=O)N1CCCC[C@H]1C(=O)O[C@H](CCc1ccc(OC)c(OC)c1)c1cccc(OCC(=O)NCCNC(=O)COc2cccc([C@@H](CCc3ccc(OC)c(OC)c3)OC(=O)[C@@H]3CCCCN3C(=O)[C@@H](CC)c3cc(OC)c(OC)c(OC)c3)c2)c1)c1cc(OC)c(OC)c(OC)c1]

48. The pharmaceutical composition of claim 34, for administration by injection, subcutaneous administration, and/or intramuscular administration.

49. The pharmaceutical composition of claim 34, for multimerization of a fusion protein and/or for eliminating cells containing a fusion protein, wherein the fusion protein comprises a polypeptide that binds to the compound of Formula A, or the pharmaceutically acceptable salt thereof.

50. A method, comprising:
combining a compound of Formula A, or a pharmaceutically acceptable salt thereof: 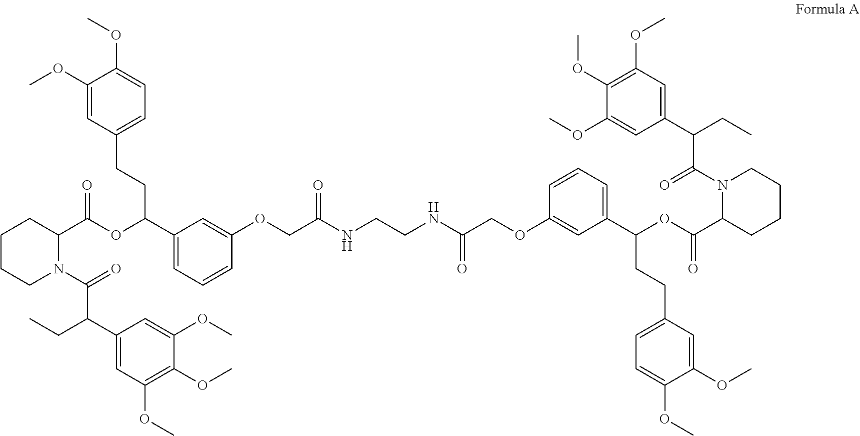 [SMILES: CCC(C(=O)N1CCCCC1C(=O)OC(CCc1ccc(OC)c(OC)c1)c1cccc(OCC(=O)NCCNC(=O)COc2cccc(C(CCc3ccc(OC)c(OC)c3)OC(=O)C3CCCCN3C(=O)C(CC)c3cc(OC)c(OC)c(OC)c3)c2)c1)c1cc(OC)c(OC)c(OC)c1]
with an oil component to provide a pharmaceutical composition for injection and/or topical administration.

51. A pharmaceutical composition, comprising:
rimiducid or a derivative thereof; and
an oil component.

52. The pharmaceutical composition of claim 51, wherein the oil component comprises medium-chain triglycerides (MCT) and polyethoxylated castor oil (PECO) and wherein the pharmaceutical composition includes polysorbate 80 (PS80).

53. The pharmaceutical composition of claim 51, wherein the oil component comprises medium-chain triglycerides (MCT) and polyethoxylated castor oil (PECO) and wherein the pharmaceutical composition includes at least one fatty acid.


SEQUENCE LISTING (7 sequences)

SEQ ID NO: 1 - protein, 108 aa, Homo sapiens
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 2 - protein, 6 aa, synthetic construct
  source 1..6: note = GS linker
SGGGSG

SEQ ID NO: 3 - protein, 286 aa, Synthetic construct
  source 1..286: note = dCasp9 (5' truncated human Casp9)
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 4 - protein, 107 aa, Synthetic construct
  source 1..107: note = human FKBP12-V36 lacking N-terminal methionine
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 5 - protein, 172 aa, Synthetic construct
  source 1..172: note = truncated MyD88
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 6 - protein, 62 aa, Synthetic construct
  source 1..62: note = truncated cytoplasmic CD40
KKVAKKPTNKAPHPKQEPQEINFPDDLPGSNTAAPVQETLHGCQPVTQEDGKESRISVQERQ

SEQ ID NO: 7 - protein, 14 aa, Synthetic construct
  source 1..14: note = myristoyl-association polypeptide
MGSSKSKPKDPSQR